(19) United States
(12) Patent Application Publication
(10) Pub. No.: US 2025/0312343 A1
(43) Pub. Date: Oct. 9, 2025

(54) COMBINATION THERAPIES USING PRMT5 INHIBITORS AND KRAS G12D INHIBITORS FOR THE TREATMENT OF CANCER

(71) Applicant: MIRATI THERAPEUTICS, INC., PRINCETON, NJ (US)

(72) Inventors: LARS DANIEL ENGSTROM, ESCONDIDO, CA (US); PETER OLSON, SAN DIEGO, CA (US); JILL HALLIN, SAN DIEGO, CA (US)

(73) Assignee: MIRATI THERAPEUTICS, INC., PRINCETON, NJ (US)

(21) Appl. No.: 19/171,408

(22) Filed: Apr. 7, 2025

Related U.S. Application Data
(60) Provisional application No. 63/631,023, filed on Apr. 8, 2024.

(51) Int. Cl. A61K 31/502; A61K 31/519; A61P 35/00

(52) U.S. Cl. CPC A61K 31/502; A61K 31/519; A61P 35/00

(57) ABSTRACT

This disclosure relates to methods of treating cancer. This disclosure further relates to treating cancer in a subject with compounds that are inhibitors are methylthioadenosine (MTA)-cooperative PRMT5 inhibitors, particularly in combination with KRAS^{G12D} inhibitors.

### BACKGROUND OF THE DISCLOSURE

### Cross Reference to Related Applications

This application claims the benefit of U.S. Provisional Application No. 63/631,023, filed Apr. 8, 2024, the entire content of which is hereby incorporated herein by reference.

### FIELD OF THE DISCLOSURE

This disclosure relates to methods of treating cancer. This disclosure further relates to treating cancer in a subject with compounds that are inhibitors of protein arginine N-methyl transferase 5 (PRMT5), particularly in combination with Kirsten rat sarcoma viral oncogene homolog (KRAS) glycine-to-aspartic acid at codon 12 (G12D) inhibitors.

### DESCRIPTION OF RELATED ART

PRMT5 is a type II arginine methyltransferase that catalyzes the transfer of a methyl group from S-adenosyl-L-methionine (SAM) to an omega-nitrogen of the guanidino function of protein L-arginine residues (omega-monomethylation) and the transfer of a second methyl group to the other omega-nitrogen, yielding symmetric dimethylarginine (sDMA). PRMT5 forms a complex with methylosome protein 50 (MEP50), which is required for substrate recognition and orientation and is also required for PRMT5-catalyzed histone 2A and histone 4 methyltransferase activity (e.g., see Ho et al. (2013) PLoS ONE 8(2): e57008).

Homozygous deletions of p16/CDKN2a are prevalent in cancer and these mutations commonly involve the co-deletion of adjacent genes, including the gene encoding methylthioadenosine phosphorylase (MTAP). It is estimated that approximately 15% of all human cancers have a homozygous deletion of the MTAP gene (e.g., see Firestone & Schramm (2017) J. Am. Chem Soc. 139(39):13754-13760).

Cells lacking MTAP activity have elevated levels of the MTAP substrate, methylthioadenosine (MTA), which is a potent inhibitor of PRMT5. Inhibition of PRMT5 activity results in reduced methylation activity and increased sensitivity of cellular proliferation to PRMT5 depletion or loss of activity. Hence, the loss of MTAP activity reduces methylation activity of PRMT5 making the cells selectively dependent on PRMT5 activity.

Kirsten Rat Sarcoma 2 Viral Oncogene Homolog (“KRas”) is a small GTPase and a member of the Ras family of oncogenes. KRas serves as a molecular switch cycling between inactive (GDP-bound) and active (GTP-bound) states to transduce upstream cellular signals received from multiple tyrosine kinases to downstream effectors to regulate a wide variety of processes, including cellular proliferation (e.g., see Alamgeer et al., (2013) Current Opin Pharmcol. 13:394-401).

The role of activated KRas in malignancy was observed over thirty years ago (e.g., see Santos et al., (1984) Science 223:661-664). Aberrant expression of KRas accounts for up to 20% of all cancers and oncogenic KRas mutations that stabilize GTP binding and lead to constitutive activation of KRas and downstream signaling have been reported in 25-30% of lung adenocarcinomas. (e.g., see Samatar and Poulikakos (2014) Nat Rev Drug Disc 13(12): 928-942 doi: 10.1038/nrd428). Single nucleotide substitutions that result in missense mutations at codons 12 and 13 of the KRas primary amino acid sequence comprise approximately 40% of these KRas driver mutations in lung adenocarcinoma. KRAS G12D mutation is present in 25.0% of all pancreatic ductal adenocarcinoma patients, 13.3% of all colorectal carcinoma patients, 10.1% of all rectal carcinoma patients, 4.1% of all non-small cell lung carcinoma patients and 1.7% of all small cell lung carcinoma patients (e.g., see The AACR Project GENIE Consortium, (2017) Cancer Discovery;7(8):818-831. Dataset Version 4).

The well-known role of KRas in malignancy and the discovery of these frequent mutations in KRas in various tumor types made KRas a highly attractive target of the pharmaceutical industry for cancer therapy. Compounds that inhibit KRas activity are still highly desirable and under investigation, including those that disrupt effectors such as guanine nucleotide exchange factors (e.g., see Sun et al., (2012) Agnew Chem Int Ed Engl. 51(25):6140-6143 doi: 10.1002/anie201201358) as well recent advances in the covalent targeting of an allosteric pocket of KRas G12C (e.g., see Ostrem et al., (2013) Nature 503:548-551 and Fell et al., (2018) ACS Med. Chem. Lett. 9:1230-1234).

Despite importance of PRMT5 on cell viability and its prevalence in cancers, effective therapies that inhibit PRMT5 have been elusive. Similarly, notwithstanding thirty years of large-scale discovery efforts to develop inhibitors of KRas for treating cancer, no KRas inhibitor has yet demonstrated sufficient safety and/or efficacy to obtain regulatory approval (e.g., see McCormick (2015) Clin Cancer Res. 21 (8):1797-1801).

For all the foregoing reasons, there is a need to develop combination therapies using PRMT5 inhibitors and KRAS^{G12D} inhibitors to treat a wide range of cancers.

### SUMMARY OF THE DISCLOSURE

One aspect of the disclosure provides methods for treating cancer in a subject. Such methods include administering to the subject a therapeutically effective amount of a KRAS^{G12D} inhibitor and a therapeutically effective amount of a PRMT5 inhibitor.

Also provided herein is a method for treating cancer in a subject in need thereof. Such methods include determining that the cancer is associated with MTAP homozygous deletion (e.g., an MTAP-associated cancer). These methods optionally further include determining that the cancer is associated with KRAS^{G12D} mutation. Such methods further include administering to the subject a therapeutically effective amount of a KRAS^{G12D} inhibitor and a therapeutically effective amount of a PRMT5 inhibitor.

These and other features and advantages of the present invention will be more fully understood from the following detailed description taken together with the accompanying claims. It is noted that the scope of the claims is defined by the recitations therein and not by the specific discussion of features and advantages set forth in the present description.

### DETAILED DESCRIPTION OF THE DISCLOSURE

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

As describe above, both MTAP^{del} and KRAS^{G12D} gene mutations are prevalent in many cancers. The present inventors have advantageously found a combination therapy to target cancers with both of these characteristics. In one aspect, the present disclosure provides a method for treating cancer in a subject, the method includes administering to the subject a therapeutically effective amount of a Kirsten rat sarcoma viral oncogene homolog glycine-to-aspartic acid at codon 12(KRAS^{G12D}) inhibitor and a therapeutically effective amount of a protein arginine N-methyl transferase 5 (PRMT5) inhibitor, wherein the PRMT5 inhibitor is methylthioadenosine (MTA)-cooperative PRMT5 inhibitor.

### Combination Therapy

In view of the present disclosure, the methods and compositions described herein can be configured by the person of ordinary skill in the art to meet the desired need. The present disclosure provides improvements in treating cancer in a subject. As used herein, the terms “subject” or “patient” are used interchangeably, refers to any animal, including mammals, and most preferably humans.

The methods provided herein may be used for the treatment of a wide variety of cancer including tumors such as lung, prostate, breast, brain, skin, cervical carcinomas, testicular carcinomas, etc. More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

In certain embodiments of the methods of the disclosure, the cancer is a MTAP-associated cancer. For example, in certain embodiments, the cancer comprises MTAP gene homozygous deletion (MTAP^{DEL}). The subject may be identified or diagnosed as having MTAP-associated cancer where, for example, MTAP^{DEL} is determined using a suitable assay or a kit. Alternatively, the subject is suspected of having MTAP-associated cancer or the subject has a clinical record indicating that the subject has MTAP-associated cancer.

In certain embodiments of the methods of the disclosure, the cancer comprises a KRAS^{G12D} gene mutation. The subject may be identified or diagnosed as having KRAS^{G12D} cancer where KRAS^{G}12D mutation is determined using a suitable assay or a kit. Alternatively, the subject is suspected of having the KRAS^{G12D} cancer or the subject has a clinical record indicating that the subject has the KRAS^{G12D} cancer.

As used herein, “KRas G12D” refers to a mutant form of a mammalian KRas protein that contains an amino acid substitution of an aspartic acid for a glycine at amino acid position 12. The assignment of amino acid codon and residue positions for human KRas is based on the amino acid sequence identified by UniProtKB/Swiss-Prot P01116: Variantp.Glyl2Asp.

As used herein, a “KRas G12D inhibitor” refers to compounds of the present invention that are represented by Formula (I), as described herein. These compounds are capable of negatively modulating or inhibiting all or a portion of the enzymatic activity of KRas G12D.

A “KRas G12D-associated disease or disorder” as used herein refers to diseases or disorders associated with or mediated by or having a KRas G12D mutation. A non-limiting example of a KRas G12D-associated disease or disorder is a KRas G12D-associated cancer.

In certain embodiments of the methods of the disclosure, the cancer may further comprise a cyclin-dependent kinase inhibitor 2A (CDKN2A) gene homozygous deletion (CDKN2A^{DEL}) The subject may be identified or diagnosed as having CDKN2A^{DEL} where the deletion is determined using a suitable assay or a kit. Alternatively, the subject is suspected of having the CDKN2A^{DEL} cancer, or the subject has a clinical record indicating that the subject has the CDKN2A^{DEL} cancer.

In some embodiments of any of the methods or uses described herein, an assay is used to determine whether the patient has MTAP^{DEL} and/or KRAS^{G12D} and/or CDKN2A^{DEL} using a sample (e.g., a biological sample or a biopsy sample such as a paraffin-embedded biopsy sample) from a subject. Such assay includes, but is not limited to, next generation sequencing, immunohistochemistry, fluorescence microscopy, break apart FISFI analysis, Southern blotting. Western blotting, FACS analysis, Northern blotting, and PCR-based amplification (e.g., RT-PCR and quantitative real-time RT-PCR). As is well known in the art, the assays are typically performed, e.g., with at least one labelled nucleic acid probe or at least one labelled antibody or antigen-binding fragment thereof.

In certain embodiments, the cancer in the methods of the disclosure is selected from lung cancer, pancreatic cancer, colon cancer, head and neck cancer, bladder cancer, esophageal cancer, lymphoma, stomach cancer, skin cancer, breast cancer, and brain cancer.

In certain embodiments, the cancer in the methods of the disclosure is selected from lung cancer, pancreatic cancer, colon cancer, head and neck cancer, esophageal cancer, and melanoma.

In certain embodiments, the cancer in the methods of the disclosure is selected from lung cancer (e.g., mesothelioma or non-small cell lung cancer (NSCLC) including adenocarcinoma and squamous cell), pancreatic cancer, colon cancer, head and neck cancer (such as squamous cell carcinoma (HNSCC)), bladder cancer, esophageal cancer, lymphoma (e.g., diffuse large B-cell lymphoma), stomach cancer, melanoma, breast cancer, and brain cancer (e.g., glioblastoma multiforme and glioma).

In certain embodiments, the cancer in the methods of the disclosure is selected from lung cancer (e.g., mesothelioma or NSCLC, including adenocarcinoma and squamous cell), pancreatic cancer, colon cancer, head and neck cancer (e.g. squamous cell carcinoma (HNSCC)), esophageal cancer, and melanoma.

In certain embodiments, the cancer in the methods of the disclosure is selected from mesothelioma, NSCLC (e.g., adenocarcinoma and squamous cell), pancreatic cancer, HNSCC, and colon cancer.

In one embodiment of the methods of the disclosure, the cancer is lung cancer. For example, the lung cancer may be NSCLC (e.g., adenocarcinoma and squamous cell) or mesothelioma.

In certain embodiment, the cancer is NSCLC.

In one embodiment of the methods of the disclosure, the cancer is pancreatic cancer.

In one embodiment of the methods of the disclosure, the cancer is colon cancer.

The PRMT5 inhibitor of the disclosure and/or the KRAS^{G12D} inhibitor of the disclosure may be provided as a pharmaceutical composition comprising a therapeutically effective amount of such inhibitor and a pharmaceutically acceptable carrier, excipient, and/or diluents. The PRMT5 inhibitor of the disclosure and/or the KRAS^{G12D} inhibitor of the disclosure may be formulated by any method well known in the art and may be prepared for administration by any route, including, without limitation, parenteral, oral, sublingual, transdermal, topical, intranasal, intratracheal, or intrarectal. In certain embodiments, the PRMT5 inhibitor of the disclosure and/or the KRAS^{G12C} inhibitor of the disclosure are administered intravenously in a hospital setting. In certain other embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, pharmaceutical compositions of the disclosure may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18^{th} Edition, ed. A. Gennaro, Mack Publishing Co., Easton, Pa., 1990.

The PRMT5 inhibitor and the KRAS^{G12D} inhibitor of the disclosure are administered in a therapeutically effective amount. As used herein, the phrase “therapeutically effective amount” or “effective amount” refers to the amount of active agent that elicits the biological or medicinal response that is being sought in a tissue, system, subject or human by a researcher, medical doctor or other clinician. In general, the therapeutically effective amount is sufficient to deliver the biological or medicinal response to the subject without causing serious toxic effects. A dose of the active agent may be in the range from about 0.01 to 300 mg/kg per day, such as 0.1 to 100 mg/kg per day, more generally 0.5 to about 25 mg/kg body weight of the recipient per day. A typical topical dosage will range from 0.01 to 3% wt/wt in a suitable carrier.

In certain embodiments of the methods of the disclosure, the therapeutically effective amount of the PRMT5 inhibitor is in the range of about 0.01 to 300 mg/kg per day. For example, in certain embodiments, the therapeutically effective amount of the PRMT5 inhibitor is in the range of about 0.1 to 100 mg/kg per day, or 25 to 100 mg/kg per day, or 50 to 100 mg/kg per day.

In certain embodiments, the therapeutically effective amount of the PRMT5 inhibitor is less than 1% of, e.g., less than 10%, or less than 25%, or less than 50% of the clinically-established therapeutic amount (e.g., such as the amount required when the PRMT5 inhibitor is administered by itself).

In some embodiments as described herein, the therapeutically effective amount of the PRMT5 inhibitor is administered once daily.

In certain embodiments of the methods of the disclosure, the therapeutically effective amount of the KRAS^{G12D} inhibitor is in the range of about 0.01 to 300 mg/kg per day. For example, in certain embodiments, the therapeutically effective amount of the KRAS^{G12D} inhibitor is in the range of about 0.1 to 100 mg/kg per day, or 0.1 to 50 mg/kg per day, or 10 to 100 mg/kg per day, or 10 to 50 mg/kg per day.

In certain embodiments, the therapeutically effective amount of the KRAS^{G12D} inhibitor is less than 1% of, e.g., less than 10%, or less than 25%, or less than 50% of the clinically-established therapeutic amount (e.g., such as the amount required when the KRAS^{G12D} inhibitor is administered by itself).

In certain embodiments as described herein, the therapeutically effective amount of the KRAS^{G12D} inhibitor is administered twice daily.

Combination therapy, in defining use of PRMT5 inhibitor and the KRAS^{G12D} inhibitor of the present disclosure, is intended to embrace administration of each agent in a sequential manner in a regimen that will provide beneficial effects of the drug combination (e.g., the PRMT5 inhibitor and the KRAS^{G12D} inhibitor of the disclosure can be formulated as separate compositions that are given sequentially), and is intended as well to embrace co-administration of these agents in a substantially simultaneous manner, such as in a single dosage form having a fixed ratio of these active agents or in multiple or a separate dosage forms for each agent. The disclosure is not limited in the sequence of administration: the PRMT5 inhibitor of the disclosure may be administered either prior to or after (i.e., sequentially), or at the same time (i.e., simultaneously) as administration of the KRAS^{G12D} inhibitor of the disclosure.

The methods of disclosure are useful as a first-line treatment. Thus, in certain embodiments of the methods of the disclosure, the subject has not previously received another first-line of therapy.

The methods of disclosure are also useful as a first-line maintenance or a second-line treatment. Thus, in certain embodiments of the methods of the disclosure, the subject has previously completed another first-line of therapy. For example, the methods of the disclosure, in certain embodiments, may provide a delay in progression and relapse of cancer in subjects that have previously completed another first-line chemotherapy. For example, in certain embodiments, the subject has previously completed a platinum- and/or taxane-based chemotherapy (e.g., carboplatin, cisplatin, oxaliplatin, paclitaxel, docetaxel, and the like). In certain embodiments of the methods of the disclosure, the subject has previously completed another first-line chemotherapy and is in partial response to such chemotherapy.

### KRAS^{G12D} Inhibitors

As described above, the methods of the disclosure include administing a KRAS^{G12D} inhibitor. A “KRAS^{G12D} inhibitor” as used herein refers to compounds of the disclosure as described herein. These compounds are capable of negatively modulating or inhibiting all or a portion of the enzymatic activity of the KRAS^{G12D}. In certain embodiments, the KRAS^{G12D} inhibitor of the disclosure is any one of the KRAS^{G12D} inhibitors disclosed in International patent publication No. WO 2022/031678, published 10 Feb. 2022, incorporated by reference in its entirety.

In certain embodiments, the KRAS^{G12D} inhibitor of the disclosure is any one of the KRAS^{G12D} inhibitors disclosed in International patent publication No. WO 2022/066646, published 31 Mar. 2022, incorporated by reference in its entirety.

In certain embodiments, the KRAS^{G12D} inhibitor of the disclosure is any one of the KRAS^{G12D} inhibitors disclosed in International patent publication No. WO 2022/098625, published 12 May 2022, incorporated by reference in its entirety.

In certain embodiments, the KRAS^{G12D} inhibitor of the disclosure is any one of the KRAS^{G12D} inhibitors disclosed in International patent publication No. WO 2021/041671 A1, published 4 Mar. 2021, incorporated by reference in its entirety.

In one aspect of the disclosure as described herein, the KRAS^{G12D} inhibitor is a compound of Formula (I):

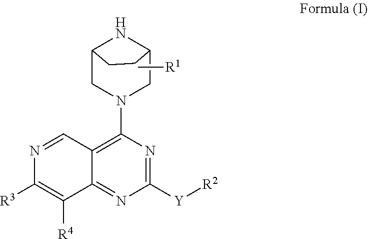

or a pharmaceutically acceptable salt thereof:
wherein:
R¹ is hydrogen, hydroxy, halogen, C1-C3 alkyl, C1-C3 cyanoalkyl, C1-C3 hydroxyalkyl, HC(═O)—, —CO₂R⁵, —CO₂N(R⁵)₂ or a 5-6 membered heteroaryl;
Y is a bond, 0 or NR¹;
R² is hydrogen, —N(R⁵)₂, heterocyclyl, C1-C6 alkyl, -L-heterocyclyl, -L-aryl, -L-heteroaryl, -L-cycloalkyl, -L-N(R⁵)₂, -L-NHC(═NH)NH₂, -L-C(O)N(R⁵)₂, -L-C1-C6 haloalkyl, -L-OR⁵, -L-(CH₂OR⁵)(CH₂).OR⁵, -L-NR⁵C(O)-aryl, -L-COOH, or -LC(═O)OC₁-C₆ alkyl, wherein the heterocyclyl and the aryl portion of -L-NR⁵C(O)-aryl and the heterocyclyl portion of -L-heterocyclyl and the cycloalkyl portion of the -L-cycloalkyl may be optionally substituted with one or more R⁶, and wherein the aryl or heteroaryl of the -L-aryl and the -L-heteroaryl may be optionally substituted with one or more R⁷;
each L is independently a C1-C4 alkylene optionally substituted with hydroxy, C1-C4 hydroxyalkyl or heteroaryl;
R³ is aryl or heteroaryl, wherein the aryl or the heteroaryl is optionally substituted with one or more R⁸;
R⁴ is hydrogen, halogen or C1-C3 alkyl;
each R⁵ is independently hydrogen or C1-C3 alkyl;
each R⁶ is independently halogen, hydroxy, C1-C3 hydroxyalkyl, C1-C3 alkyl, C1-C3 haloalkyl, C1-C3 alkoxy, cyano, -Q-phenyl, -Q-phenylSO₂F, —NHC(O)phenyl, —NHC(O)phenylSO₂F, C1-C3 alkyl substituted pyrazolyl, araC1-C3 alkyl-, tert-butyldimethylsilyloxyCH₂—, —N(R⁵)₂, (C1-C3 alkoxy)C1-C3 alkyl-, (C1-C3 alkyl)C(═O), oxo, (C1-C3 haloalkyl)C(═O)—, —SO₂F, (C1-C3 alkoxy)C1-C3 alkoxy, —CH₂OC(O)N(R⁵)₂, —CH₂NHC(O)OC₁-C6 alkyl, —CH₂NHC(O)N(R⁵)₂, —CH₂NHC(O)C1-C6 alkyl, —CH₂(pyrazolyl), —CH₂NHSO₂C1-C6 alkyl, —CH₂OC(O)heterocyclyl, —OC(O)N(R⁵)₂, —OC(O)NH(C1-C3 alkyl)O(C1-C3 alkyl), —OC(O)NH(C1-C3 alkyl)O(C1-C3 alkyl)phenyl(C1-C3 alkyl)N(CH₃)₂, —OC(O)NH(C1-C3 alkyl)O(C1-C3 alkyl)phenyl or —OC(O)heterocyclyl, —CH₂ heterocyclyl, wherein the phenyl of —NHC(O)phenyl or —OC(O)NH(C1-C3 alkyl)O(C1-C3 alkyl)phenyl is optionally substituted with —C(O)H or OH and wherein the heterocyclyl of —CH₂ heterocyclyl is optionally substituted with oxo;
Q is a bond or 0;
each R⁷ is independently halogen, hydroxy, HC(═O)—, C1-C4 alkyl, C1-C4 alkoxy, C1-C4 haloalkyl, C1-C4 hydroxyalkyl, or —N(R⁵)₂; and each R⁸ is independently halogen, cyano, hydroxy, C1-C4 alkyl, —S-C1-C3 alkyl, C2-C4 alkenyl, C2-C4 alkynyl, C2-C4 hydroxyalkynyl, C1-C3 cyanoalkyl, triazolyl, C1-C3 haloalkyl, —O— C1-C3 haloalkyl, —S-C1-C3 haloalkyl, C1-C3 alkoxy, hydroxyC₁-C3 alkyl, —CH₂C(═O)N(R⁵)₂, -C3-C4 alkynyl(NR⁵)₂, —N(R⁵)₂, deuteroC2-C4 alkynyl, (C1-C3 alkoxy)haloC₁-C3 alkyl-, or C3-C6 cycloalkyl wherein said C3-C6 cycloalkyl is optionally substituted with halogen or C1-C3 alkyl.

In one embodiment of the compounds of Formula (I), R¹ is halogen, hydroxy, C1-C3 alkyl, C1-C3 cyanoalkyl, C1-C3 hydroxyalkyl, HC(═O)—, —CO₂R⁵, or —CO₂N(R′)₂.

In certain embodiments, R¹ is hydrogen.

In certain embodiments, R¹ is hydroxy.

In other embodiments, R¹ is —CO₂R⁵. In certain embodiments, R⁵ is hydrogen. In other embodiments, R⁵ is C1-C3 alkyl.

In another embodiment, R¹ is —C(O)₂N(R⁵)₂. In certain embodiments, each R⁵ is hydrogen, each R⁵ is an independently selected C1-C3 alkyl, or one R⁵ is hydrogen and the second R⁵ is C1-C3 alkyl.

In one embodiment of the compounds of Formula (I), Y is a bond.

In one embodiment of the compounds of Formula (I), Y is a bond and R² is hydrogen, —N(R⁵)₂, or heterocyclyl optionally substituted with one or more R⁶.

In certain embodiments of the compounds of Formula (I), R² is —N(R⁵)₂. In one embodiment, each R⁵ is hydrogen. In one embodiment, each R⁵ is an independently selected C1-C3 alkyl. In one embodiment, one R⁵ is hydrogen and the second R⁵ is C1-C3 alkyl. In certain embodiments, Y is a bond and R² is —N(R⁵)₂.

In other embodiments, R² is heterocyclyl. In one embodiment R² is heterocyclyl and the heterocyclyl is azetidinyl, pyrrolidinyl, tetrahydro-2H-thiopyran 1,1-dioxide or 1,6λ²-diazaspiro[3.3]heptanyl. In certain embodiments, Y is a bond and R² is heterocyclyl.

In certain embodiments, the heterocyclyl is azetidinyl substituted with one R⁶. In certain embodiments, the heterocyclyl is azetidinyl substituted with one R⁶, wherein R⁶ is hydroxy, hydroxyalkyl, or —N(R⁵)₂. In certain embodiments, the heterocyclyl is azetidinyl substituted with two R⁶ groups independently selected from —N(R⁵)₂ and C1-C3 alkyl. In certain embodiments, Y is a bond and the heterocyclyl is azetidinyl substituted with one R⁶, wherein R⁶ is hydroxy, hydroxyalkyl, or —N(R⁵)₂. In certain embodiments, Y is a bond and the heterocyclyl is azetidinyl substituted with two R⁶ groups independently selected from —N(R⁵)₂ and C1-C3 alkyl.

In one embodiment of the compounds of Formula (I), Y is O.

In one embodiment, Y is O and R² is C1-C6 alkyl, -L-heterocyclyl optionally substituted with one or more R⁶, -L-heteroaryl, wherein the heteroaryl portion is optionally substituted with one or more R⁷, -L-aryl, wherein the aryl portion is optionally substituted with one or more R⁷, -L-cycloalkyl, wherein the cycloalkyl portion is optionally substituted with one or more R⁶, -L-N(R⁵)₂, -L-NC(═NH)—NH₂, -L-C(O)N(R⁵)₂, -L-C1-C6 haloalkyl, -L-COR⁵, -L-(CH₂OR⁵)(CH₂)ₙOR⁵, -L-NR⁵C(O)-aryl.

In one embodiment of the compounds of Formula (I), Y is O and R² is C1-C6 alkyl. In certain embodiments, the C1-C6 alkyl is methyl, ethyl, isopropyl or isobutyl.

In one embodiment of the compounds of Formula (I), Y is O and R² is -L-heterocyclyl optionally substituted with one or more R⁶.

In one embodiment, Y is O and R² is heterocyclyl wherein the heterocyclyl is tetrahydropyranyl optionally substituted with two halogens. In certain embodiment, the two halogens are both fluoro.

In another embodiment, Y is O and R² is -L-heterocyclyl wherein L is methylene and the heterocyclyl is hexahydro-1H-pyrrolizinyl, hexahydro-3H-pyrrolizin-3-one, hexahydro-1H-pyrrolo[2,1-c][1,4]oxazinyl, octahydroindolizinyl, hexahydropyrrolizine 4(1H)-oxide, azetidinyl, pyrrolidinyl, pyrrolidin-2-one, oxetanyl, piperidinyl, 1-azabicyclo[2.2.1]heptanyl, morpholinyl, oxa-5-azabicyclo[2.2.1]heptan-5-yl, thiopyranyl, 6-oxa-2λ²-azaspiro[3.4]octanyl, 7-oxa-2λ²-azaspiro[3.5]nonanyl, 2′,3′-dihydrospiro[cyclopropane-1,1′-indenyl], (2S)-1-azabicyclo[2.2.1]heptan-2-yl or tetrahydrofuranyl.

In certain embodiments, Y is O and R² is -L-heterocyclyl wherein L is methylene and the heterocyclyl is hexahydro-1H-pyrrolizinyl.

In certain embodiments, Y is O and R² is -L-heterocyclyl wherein L is methylene and the heterocyclyl is hexahydro-1H-pyrrolizinyl is optionally substituted with one R⁶, wherein R⁶ is halogen, hydroxy, hydroxyalkyl, C1-C3 haloalkyl, C1-C3 alkyl, C1-C3 alkoxy, phenyl, tert-butyldimethylsilyloxyCH₂- or pyrazolyl, wherein the pyrazolyl is optionally substituted with C1-C3 alkyl. In one embodiment, the C1-C3 haloalkyl is chloromethyl. In another embodiment, the pyrazolyl is substituted with C1-C3 alkyl. In other embodiments, the hexahydro-1H-pyrrolizinyl is substituted with two R⁶ groups, wherein each R⁶ is an independently selected C1-C3 alkyl. In certain embodiments, the heterocyclyl is hexahydro-1H-pyrrolizinyl which is unsubstituted.

In certain embodiments, Y is O and R² is -L-heterocyclyl wherein L is methylene and the heterocyclyl is azetidinyl substituted with one R⁶, wherein R⁶ is C1-C3 alkyl.

In certain embodiments, Y is O and R² is -L-heterocyclyl wherein L is methylene and the heterocyclyl is pyrrolidinyl substituted with one R⁶, wherein R⁶ is C1-C3 hydroxyalkyl, C1-C3 haloalkyl, C1-C3 alkyl, C1-C3 alkoxy, C1-C3 aralkyl, or -Q-phenyl, wherein Q is O, and —NHC(O)phenyl. In one embodiment, the phenyl group of the -Q-phenyl is substituted with SO₂F. In another embodiment, the phenyl group of the —NHC(O)phenyl is substituted with SO₂F. In one embodiment, the C1-C3 aralkyl is benzyl.

In other embodiments, Y is O and R² is -L-heterocyclyl wherein L is methylene and the pyrrolidinyl is substituted with two R⁶ groups, wherein one R⁶ is C1-C3 alkyl and the other R⁶ is C1-C3 alkoxy or halogen.

In certain embodiments, Y is O and R² is -L-heterocyclyl wherein L is methylene and the heterocyclyl is pyrrolidin-2-one substituted with one R⁶, wherein R⁶ is C1-C3 alkyl.

In certain embodiments, Y is O and R² is -L-heterocyclyl wherein L is methylene and the heterocyclyl is piperidinyl substituted with one R⁶, wherein R⁶ is acetyl, (C1-C3 alkoxy)C1-C3 alkoxy, or —C(O)CH₂C1.

In certain embodiments, Y is O and R² is -L-heterocyclyl wherein L is methylene and the heterocyclyl is (2S)-1-azabicyclo[2.2.1]heptan-2-yl.

In one embodiment of the compounds of Formula (I), Y is 0, R² is -L-heterocyclyl wherein L is ethylene or propylene and the heterocyclyl is morpholinyl or oxa-5-azabicyclo[2.2.1]heptan-5-yl.

In one embodiment of the compounds of Formula (I), Y is O and R² is -L-heteroaryl, wherein the heteroaryl portion is optionally substituted with one or more R⁷. In certain embodiments, L is ethylene and the heteroaryl is benzimidazolyl, optionally substituted with one or more R⁷. In one embodiment, R⁷ is C1-C4 alkyl.

In certain embodiments, Y is O and R² is -L-heteroaryl.

In certain embodiments, Y is O and R² is -L-heteroaryl, wherein L is methylene or ethylene.

In certain embodiments, Y is O and R² is -L-heteroaryl, wherein L is methylene or ethylene and the heteroaryl is pyridyl, pyrazolyl, imidazolyl, triazolyl, 4,5,6,7-tetrahydro-1H-indazolyl, benzimidazolyl, imidazo[1,2-a]pyridinyl, or pyrimidinyl.

In certain embodiments, Y is O and R² is -L-heteroaryl, wherein the heteroaryl is pyridyl substituted with one R⁷. In certain embodiments, Y is O and R² is -L-heteroaryl, wherein the heteroaryl is pyridyl substituted with one R⁷ wherein R⁷ is halogen, C1-C4 haloalkyl, C1-C4 hydroxyalkyl, C1-C4 alkyl, —N(R⁵)₂, or C1-C4 alkoxy.

In certain embodiments, Y is O and R² is -L-heteroaryl, wherein L is methylene or ethylene and the heteroaryl is pyrazolyl substituted with one R⁷. In certain embodiments, Y is O and R² is -L-heteroaryl, wherein L is methylene or ethylene and the heteroaryl is pyrazolyl substituted with one R⁷ wherein R⁷ is halogen, C1-C4 haloalkyl, C1-C4 hydroxyalkyl, C1-C4 alkyl, alkoxy or —N(R⁵)₂.

In certain embodiments, Y is O and R² is -L-heteroaryl, wherein L is methylene or ethylene and the heteroaryl is imidazolyl substituted with one R⁷. In certain embodiments, Y is O and R² is -L-heteroaryl, wherein L is methylene or ethylene and the heteroaryl is imidazolyl substituted with one R⁷ wherein R⁷ is C1-C4 alkyl, C1-C4 haloalkyl, or C1-C4 hydroxyalkyl.

In certain embodiments, Y is O and R² is -L-heteroaryl, wherein L is methylene or ethylene and the heteroaryl is triazolyl substituted with one R⁷. In certain embodiments, Y is O and R² is -L-heteroaryl, wherein L is methylene or ethylene and the heteroaryl is triazolyl substituted with one R⁷, wherein R⁷ is C1-C4 alkyl.

In one embodiment of the compounds of Formula (I), Y is O and R² is -L-aryl, wherein the aryl portion is optionally substituted with one or more R⁷. In certain embodiments, L is ethylene and the aryl is phenyl. In one embodiment, the phenyl is substituted with one R⁷. In one embodiment, the phenyl is substituted with one R⁷, wherein R⁷ is halogen. In one embodiment, the phenyl is substituted with two R⁷ groups. In one embodiment, the phenyl is substituted with two R⁷ groups. In one embodiment, the phenyl is substituted with two R⁷ groups wherein one R⁷ is hydroxy and one R⁷ is HC(═O)—.

In one embodiment of the compounds of Formula (I), Y is O and R² is -L-cycloalkyl, wherein the cycloalkyl portion is optionally substituted with one or more R⁶. In one embodiment, L is methylene. In one embodiment, the cycloalkyl is cyclopropyl, cyclobutyl, cyclopentyl or cyclohexyl. In certain embodiments, the cyclopropyl and cyclopentyl are each substituted with one R⁶. In certain embodiments, the cyclopropyl and cyclopentyl are each substituted with one R⁶, wherein R⁶ is haloalkyl. In certain embodiments, the cyclobutyl and cyclohexyl are each substituted with two R⁶ groups. In certain embodiments, the cyclobutyl and cyclohexyl are each substituted with two R⁶ groups, wherein each R⁶ group is halogen.

In one embodiment of the compounds of Formula (I), Y is 0, and R² is -L-N(R⁵)₂. In certain embodiments, L is ethylene. In certain embodiments, R⁵ is C1-C3 alkyl.

In one embodiment of the compounds of Formula (I), Y is 0, and R² is -L-NC(═NH)—NH₂. In certain embodiments, L is ethylene or propylene.

In one embodiment of the compounds of Formula (I), Y is 0, and R² is -L-C(O)N(R⁵)₂. In certain embodiments, L is ethylene and each R⁵ is C1-C3 alkyl.

In one embodiment of the compounds of Formula (I), Y is 0, and R² is -L-C1-C6 haloalkyl. In certain embodiments, L is methylene. In certain embodiments, the haloalkyl is 1,1,3,3-tetrafluoropropanyl or trifluoromethyl. In other embodiments, L is ethylene or propylene and the haloalkyl is trifluoromethyl.

In one embodiment of the compounds of Formula (I), Y is 0, and R² is -L-COR⁵. In certain embodiments, L is propylene and R⁵ is hydrogen or C1-C3 alkyl. In certain embodiments, L is propylene that is substituted with hydroxy, hydroxyalkyl or heteroaryl and R⁵ is hydrogen or C1-C3 alkyl. In one embodiment, the heteroaryl is pyridyl.

In one embodiment of the compounds of Formula (I), Y is 0, and R² is -L-(CH₂OR⁵)(CH₂)ₙOR⁵. In certain embodiments, L is methylene, each R⁵ is independently hydrogen or C1-C3 alkyl, and n is one or two.

In one embodiment of the compounds of Formula (I), Y is 0, and R² is -L-NR⁵C(O)-aryl. In certain embodiments, L is methylene, R⁵ is hydrogen. In one embodiment the aryl is phenyl.

In one embodiment, the phenyl is substituted with one R⁶, wherein R⁶ is —SO₂F.

In one embodiment of the compounds of Formula (I), R³ is aryl optionally substituted with one or more R⁸. In certain embodiments, the aryl is selected from the group consisting of phenyl, naphthyl, 1,2,3,4-tetrahydronaphthalenyl and 2,3-dihydro-1H-indenyl, wherein each is optionally substituted with one or more R⁸.

In one embodiment, the aryl is phenyl substituted with one or more R⁸ groups. In one embodiment, the aryl is phenyl substituted with one or more R⁸ groups independently selected from halogen, C1-C3 haloalkyl and —O-C1-C3 haloalkyl. In certain embodiments the phenyl is substituted with two R⁸ groups. In certain embodiments the phenyl is substituted with two R⁸ groups, wherein the two R⁸ groups are two independently selected C1-C3 haloalkyl groups, or —O-C1-C3 haloalkyl and halogen.

In one embodiment, the aryl is 2,3-dihydro-1H-indenyl optionally substituted with one or more R¹. In one embodiment, the aryl is 2,3-dihydro-1H-indenyl optionally substituted with one R⁸. In one embodiment, R⁸ is C1-C alkyl.

In one embodiment, the aryl is naphthyl substituted with one or more R⁸ groups. In one embodiment, the aryl is naphthyl substituted with one or more R¹ groups independently selected from halogen, cyano, hydroxy, C1-C3 alkyl, —S-C1-C3 alkyl, C2-C4 alkenyl, C2-C4 alkynyl, C2-C4 hydroxyalkynyl, C1-C3 cyanoalkyl, triazolyl, C1-C3 haloalkyl and —O-C1-C3 haloalkyl.

In one embodiment, the aryl is naphthyl substituted with hydroxy. In one embodiment, the aryl is naphthyl substituted with halogen. In certain embodiments, the halogen is chlorine, fluorine or bromine. In other embodiments, the halogen is chlorine.

In one embodiment, the aryl is naphthyl substituted with C1-C3 alkyl, wherein the C1-C3 alkyl is methyl or ethyl.

In one embodiment, the aryl is naphthyl substituted with C2-C4 alkenyl. In certain embodiments, the C2-C4 alkenyl is prop-2-enyl.

In one embodiment, the aryl is naphthyl substituted with C2-C4 alkynyl. In certain embodiments, the C2-C4 alkynyl is ethyne or prop-2-ynyl.

In one embodiment, the aryl is naphthyl substituted with one or two R⁸, wherein each R⁸ is halogen, cyano, hydroxy, C1-C3 alkyl, —S-C1-C3 alkyl, C2-C4 alkenyl, C2-C4 alkynyl, C2-C4 hydroxyalkynyl, C1-C3 cyanoalkyl, or triazolyl. In one embodiment, the aryl is naphthyl substituted with two R¹ groups independently selected from halogen, hydroxy, C1-C3 alkyl and C2-C4 alkynyl.

In one embodiment of the compounds of Formula (I), R³ is heteroaryl optionally substituted with one or more R¹. In one embodiment, the heteroaryl is isoquinolinyl, indazolyl, or benzo[d][1,3]dioxolyl optionally substituted with one or more R⁸. In one embodiment, the heteroaryl is indazolyl optionally substituted with one or more R⁸. In one embodiment, the heteroaryl is indazolyl optionally substituted with C1-C3 alkyl. In other embodiments, the heteroaryl is isoquinolinyl optionally substituted with one or more R⁸. In other embodiments, the heteroaryl is isoquinolinyl optionally substituted with halogen or C2-C4 alkynyl. In certain embodiments, the heteroaryl is benzo[d][1,3]dioxolyl optionally substituted with two R⁸ groups. In certain embodiments, the heteroaryl is benzo[d][1,3]dioxolyl optionally substituted with two R⁸ groups, wherein each R⁸ group is an independently selected halogen.

In one embodiment, the two halogens are gem-difluoro substitutions.

In one embodiment of the compounds of Formula (I), R⁴ is hydrogen.

In one embodiment of the compounds of Formula (I), R⁴ is halogen. In one embodiment, R⁴ is fluorine. In one embodiment, R⁴ is chlorine.

In one embodiment of the compounds of Formula (I), R⁴ is C1-C3 alkyl. In one embodiment, R⁴ is methyl.

Nonlimiting examples of compounds of Formula (I) are selected from the group consisting of:

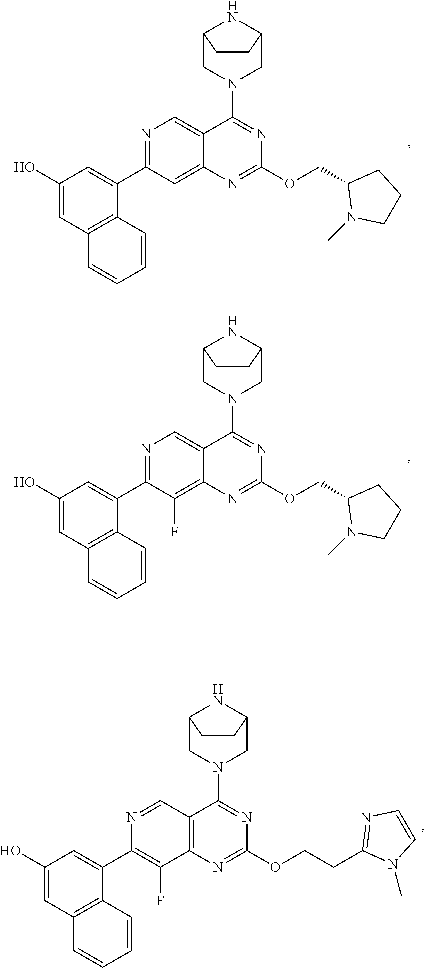 [SMILES: CN1CCC[C@H]1COc1nc(N2CC3CCC(C2)N3)c2cnc(-c3cc(O)cc4ccccc34)cc2n1 ; CN1CCC[C@H]1COc1nc(N2CC3CCC(C2)N3)c2cnc(-c3cc(O)cc4ccccc34)c(F)c2n1 ; Cn1ccnc1CCOc1nc(N2CC3CCC(C2)N3)c2cnc(-c3cc(O)cc4ccccc34)c(F)c2n1]

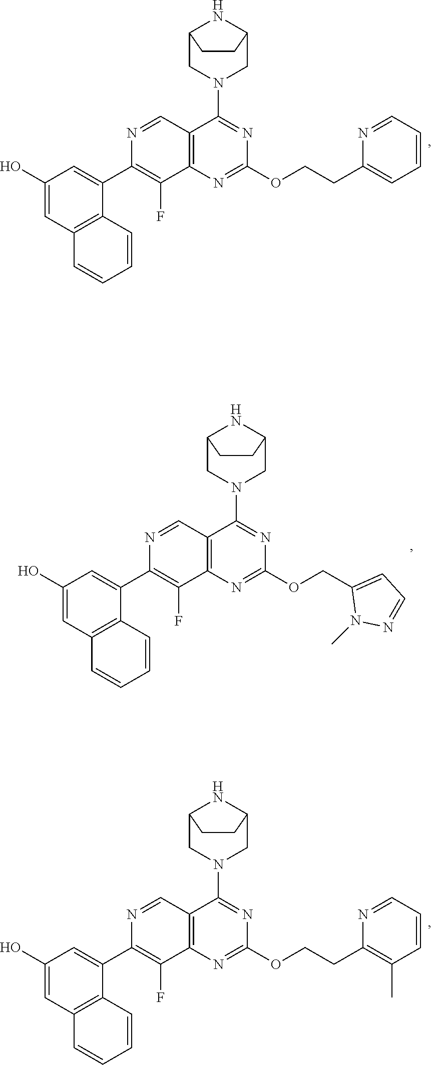 [SMILES: Oc1cc(-c2ncc3c(N4CC5CCC(C4)N5)nc(OCCc4ccccn4)nc3c2F)c2ccccc2c1 ; Cn1nccc1COc1nc(N2CC3CCC(C2)N3)c2cnc(-c3cc(O)cc4ccccc34)c(F)c2n1 ; Cc1cccnc1CCOc1nc(N2CC3CCC(C2)N3)c2cnc(-c3cc(O)cc4ccccc34)c(F)c2n1]

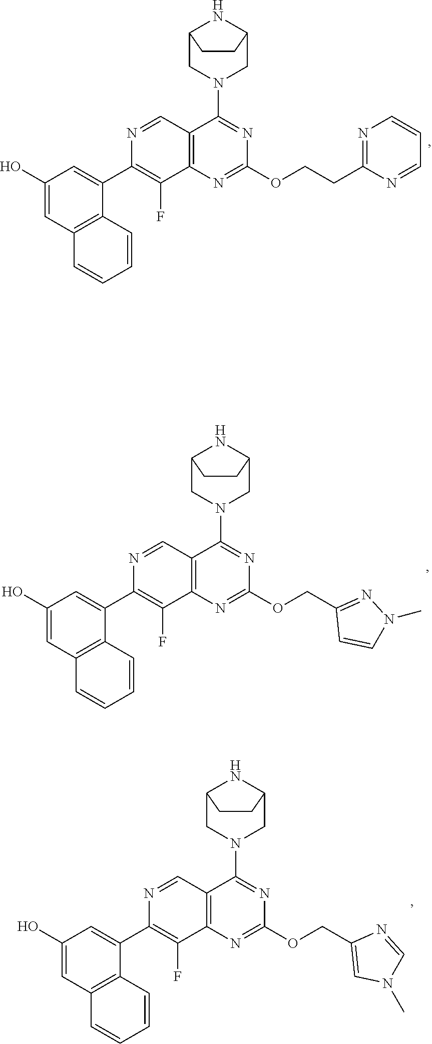 [SMILES: Oc1cc(-c2ncc3c(N4CC5CCC(C4)N5)nc(OCCc4ncccn4)nc3c2F)c2ccccc2c1 ; Cn1ccc(COc2nc(N3CC4CCC(C3)N4)c3cnc(-c4cc(O)cc5ccccc45)c(F)c3n2)n1 ; Cn1cnc(COc2nc(N3CC4CCC(C3)N4)c3cnc(-c4cc(O)cc5ccccc45)c(F)c3n2)c1]

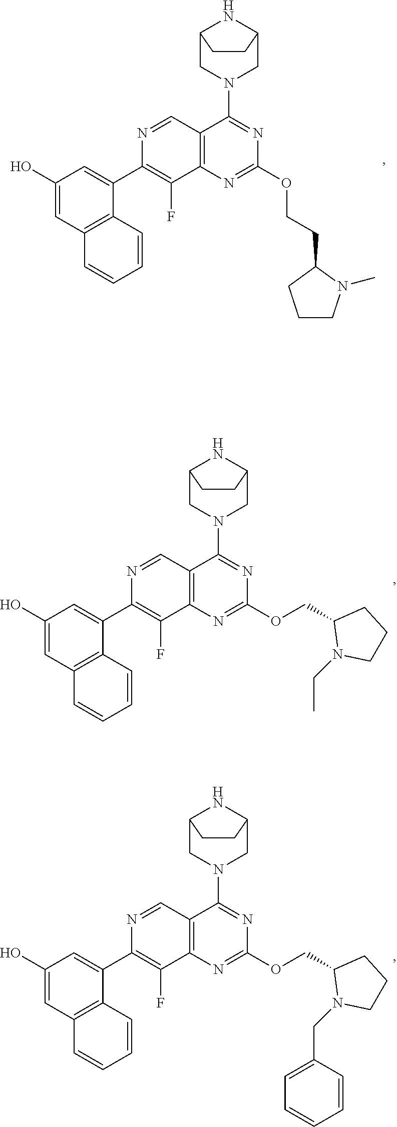 [SMILES: CN1CCC[C@H]1CCOc1nc(N2CC3CCC(C2)N3)c2cnc(-c3cc(O)cc4ccccc34)c(F)c2n1 ; CCN1CCC[C@H]1COc1nc(N2CC3CCC(C2)N3)c2cnc(-c3cc(O)cc4ccccc34)c(F)c2n1 ; Oc1cc(-c2ncc3c(N4CC5CCC(C4)N5)nc(OC[C@@H]4CCCN4Cc4ccccc4)nc3c2F)c2ccccc2c1]

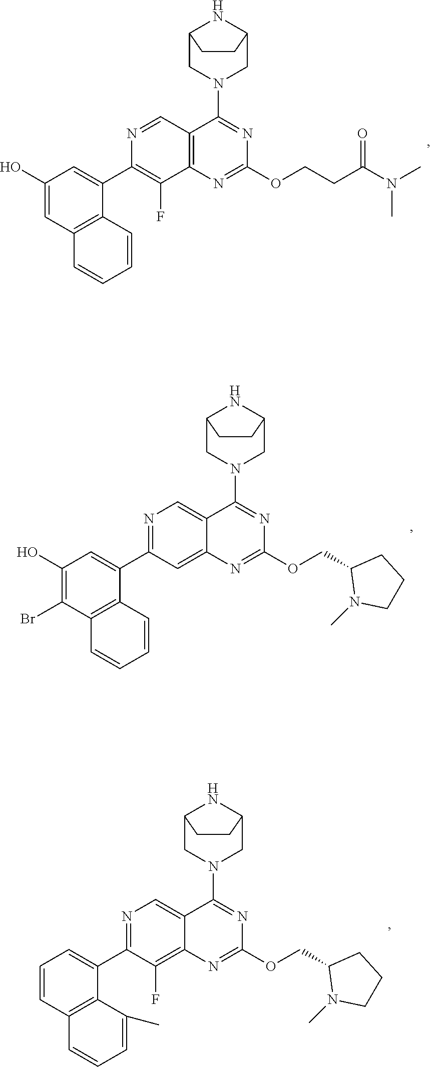 [SMILES: CN(C)C(=O)CCOc1nc(N2CC3CCC(C2)N3)c2cnc(-c3cc(O)cc4ccccc34)c(F)c2n1 ; CN1CCC[C@H]1COc1nc(N2CC3CCC(C2)N3)c2cnc(-c3cc(O)c(Br)c4ccccc34)cc2n1 ; Cc1cccc2cccc(-c3ncc4c(N5CC6CCC(C5)N6)nc(OC[C@@H]5CCCN5C)nc4c3F)c12]

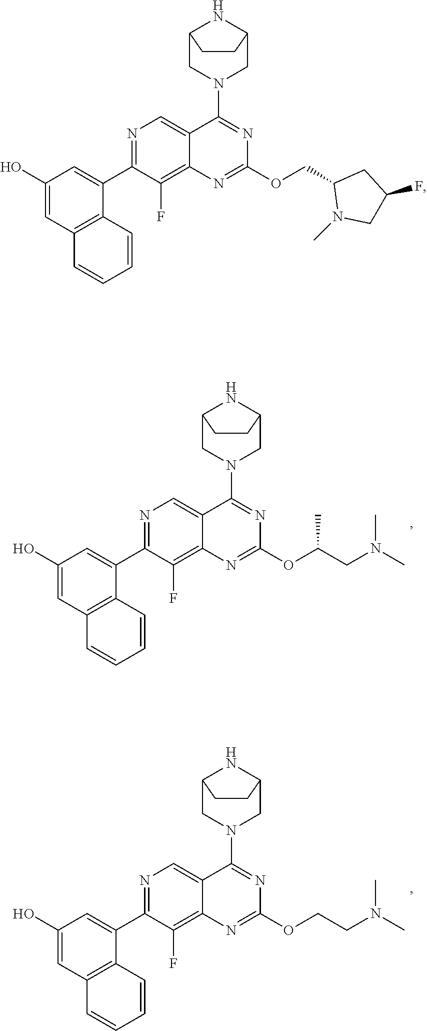 [SMILES: CN1C[C@H](F)C[C@H]1COc1nc(N2CC3CCC(C2)N3)c2cnc(-c3cc(O)cc4ccccc34)c(F)c2n1 ; C[C@H](CN(C)C)Oc1nc(N2CC3CCC(C2)N3)c2cnc(-c3cc(O)cc4ccccc34)c(F)c2n1 ; CN(C)CCOc1nc(N2CC3CCC(C2)N3)c2cnc(-c3cc(O)cc4ccccc34)c(F)c2n1]

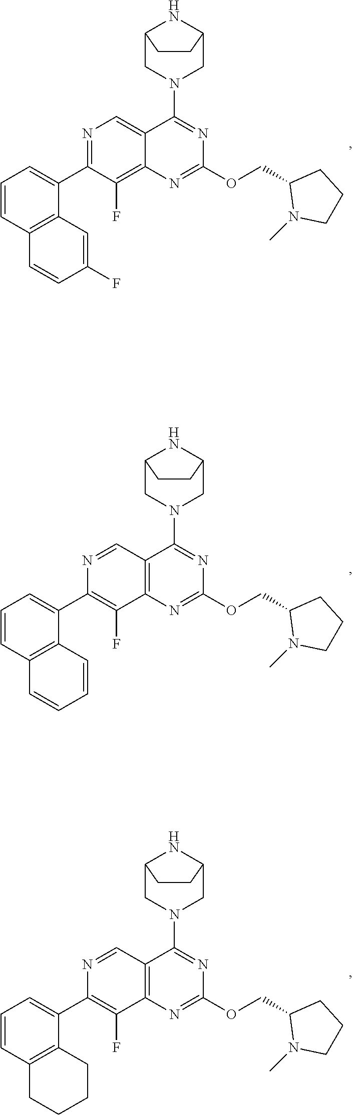 [SMILES: CN1CCC[C@H]1COc1nc(N2CC3CCC(C2)N3)c2cnc(-c3cccc4ccc(F)cc34)c(F)c2n1 ; CN1CCC[C@H]1COc1nc(N2CC3CCC(C2)N3)c2cnc(-c3cccc4ccccc34)c(F)c2n1 ; CN1CCC[C@H]1COc1nc(N2CC3CCC(C2)N3)c2cnc(-c3cccc4c3CCCC4)c(F)c2n1]

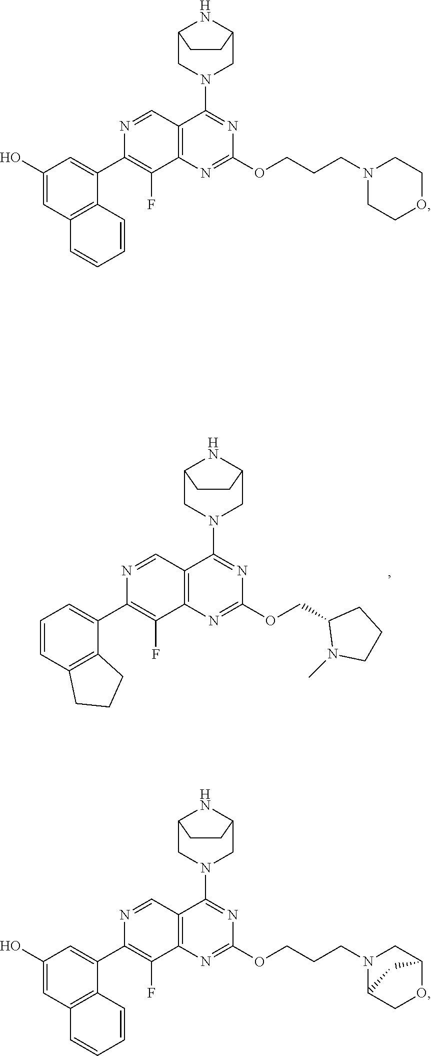

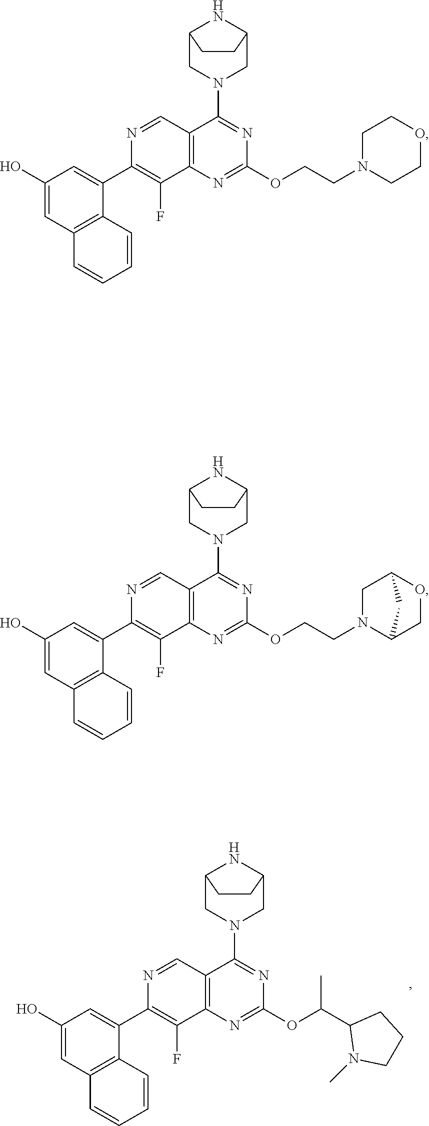

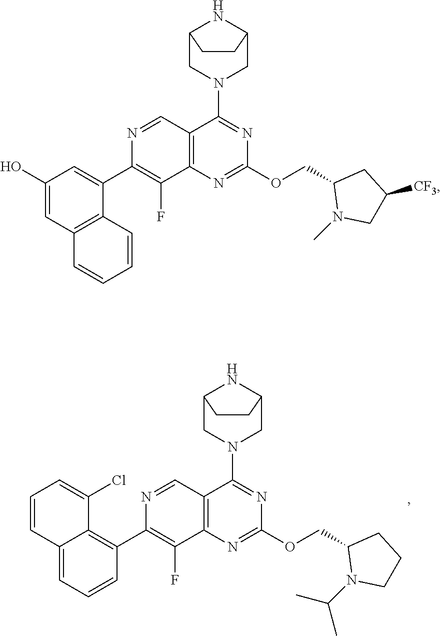 [SMILES: CN1C[C@H](C(F)(F)F)C[C@H]1COc1nc(N2CC3CCC(C2)N3)c2cnc(-c3cc(O)cc4ccccc34)c(F)c2n1 ; CC(C)N1CCC[C@H]1COc1nc(N2CC3CCC(C2)N3)c2cnc(-c3cccc4cccc(Cl)c34)c(F)c2n1]

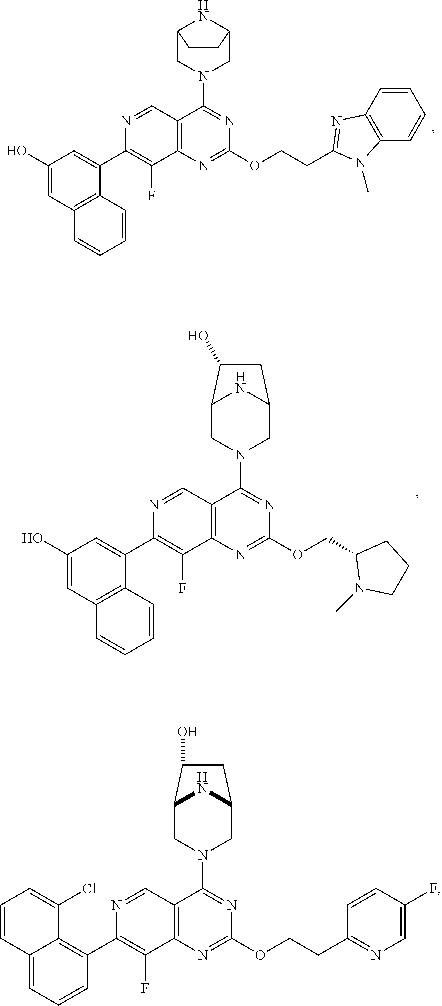

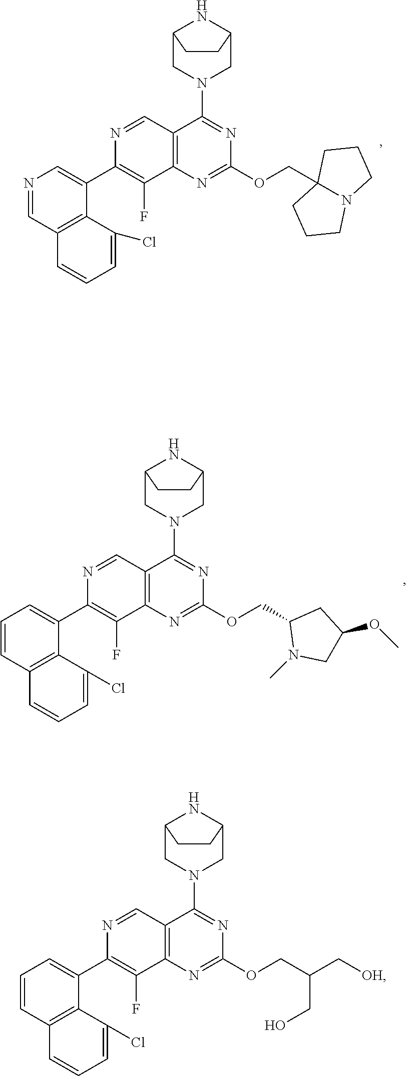 [SMILES: Fc1c(-c2cncc3cccc(Cl)c23)ncc2c(N3CC4CCC(C3)N4)nc(OCC34CCCN3CCC4)nc12 ; CO[C@@H]1C[C@@H](COc2nc(N3CC4CCC(C3)N4)c3cnc(-c4cccc5cccc(Cl)c45)c(F)c3n2)N(C)C1 ; OCC(CO)COc1nc(N2CC3CCC(C2)N3)c2cnc(-c3cccc4cccc(Cl)c34)c(F)c2n1]

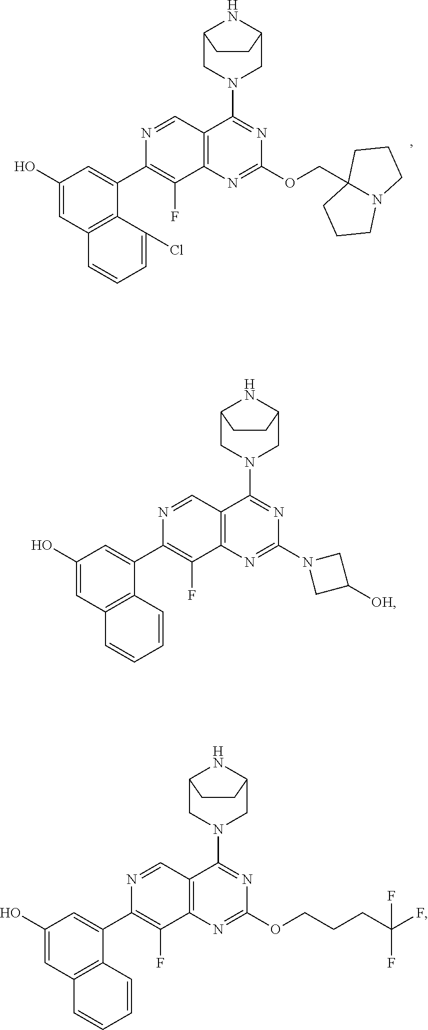 [SMILES: Oc1cc(-c2ncc3c(N4CC5CCC(C4)N5)nc(OCC45CCCN4CCC5)nc3c2F)c2c(Cl)cccc2c1 ; Oc1cc(-c2ncc3c(N4CC5CCC(C4)N5)nc(N4CC(O)C4)nc3c2F)c2ccccc2c1 ; Oc1cc(-c2ncc3c(N4CC5CCC(C4)N5)nc(OCCCC(F)(F)F)nc3c2F)c2ccccc2c1]

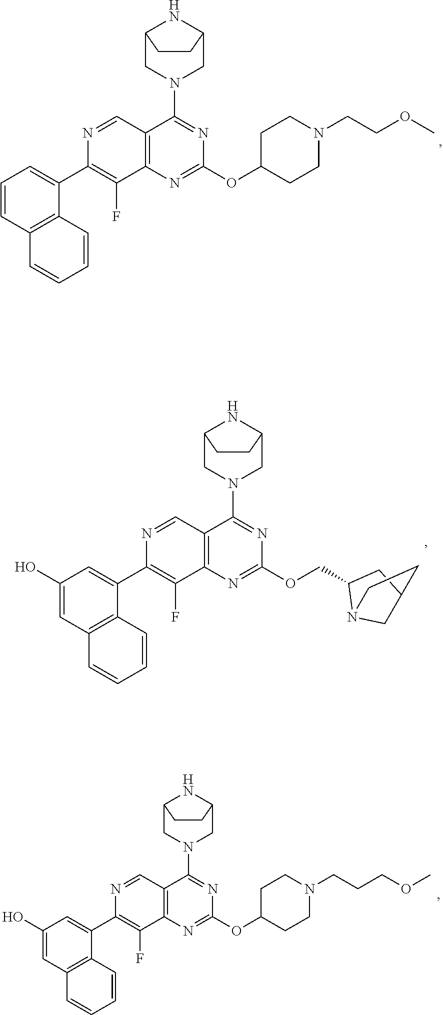 [SMILES: COCCN1CCC(Oc2nc(N3CC4CCC(C3)N4)c3cnc(-c4cccc5ccccc45)c(F)c3n2)CC1 ; Oc1cc(-c2ncc3c(N4CC5CCC(C4)N5)nc(OC[C@@H]4CC5CCN4C5)nc3c2F)c2ccccc2c1 ; COCCCN1CCC(Oc2nc(N3CC4CCC(C3)N4)c3cnc(-c4cc(O)cc5ccccc45)c(F)c3n2)CC1]

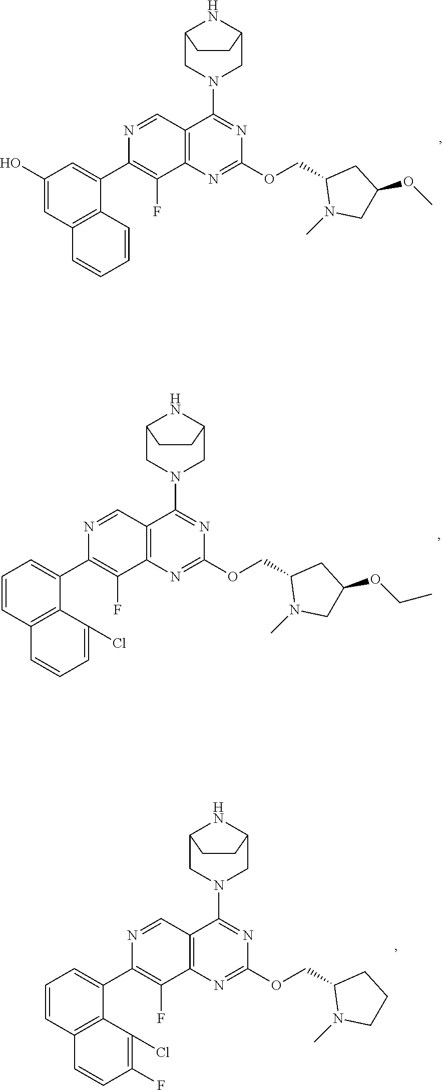 [SMILES: CO[C@@H]1C[C@@H](COc2nc(N3CC4CCC(C3)N4)c3cnc(-c4cc(O)cc5ccccc45)c(F)c3n2)N(C)C1 ; CCO[C@@H]1C[C@@H](COc2nc(N3CC4CCC(C3)N4)c3cnc(-c4cccc5cccc(Cl)c45)c(F)c3n2)N(C)C1 ; CN1CCC[C@H]1COc1nc(N2CC3CCC(C2)N3)c2cnc(-c3cccc4ccc(F)c(Cl)c34)c(F)c2n1]

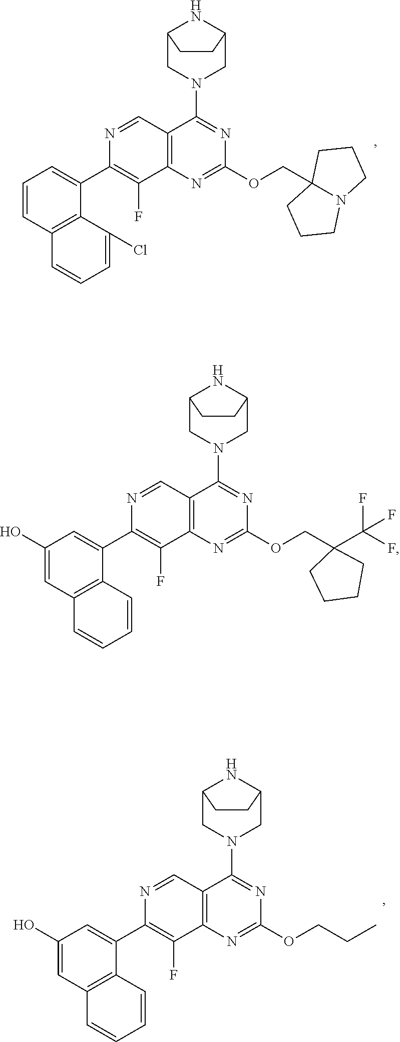 [SMILES: Fc1c(-c2cccc3cccc(Cl)c23)ncc2c(N3CC4CCC(C3)N4)nc(OCC34CCCN3CCC4)nc12 ; Oc1cc(-c2ncc3c(N4CC5CCC(C4)N5)nc(OCC4(C(F)(F)F)CCCC4)nc3c2F)c2ccccc2c1 ; CCCOc1nc(N2CC3CCC(C2)N3)c2cnc(-c3cc(O)cc4ccccc34)c(F)c2n1]

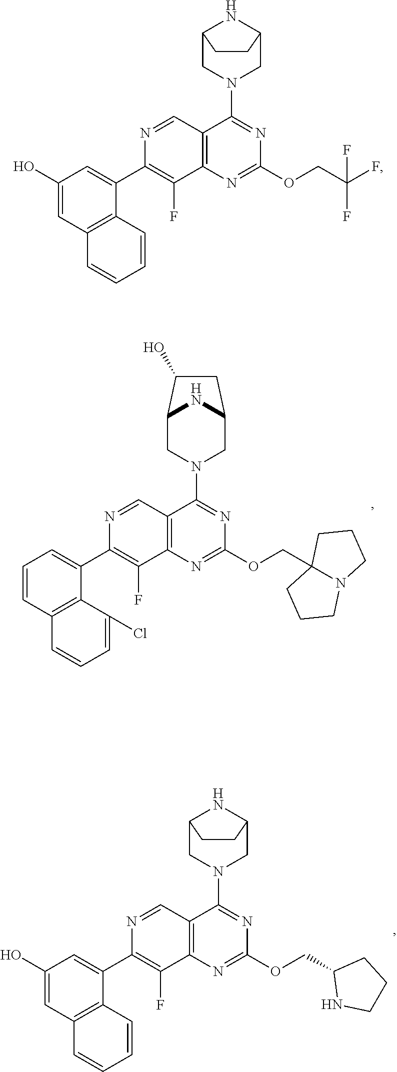

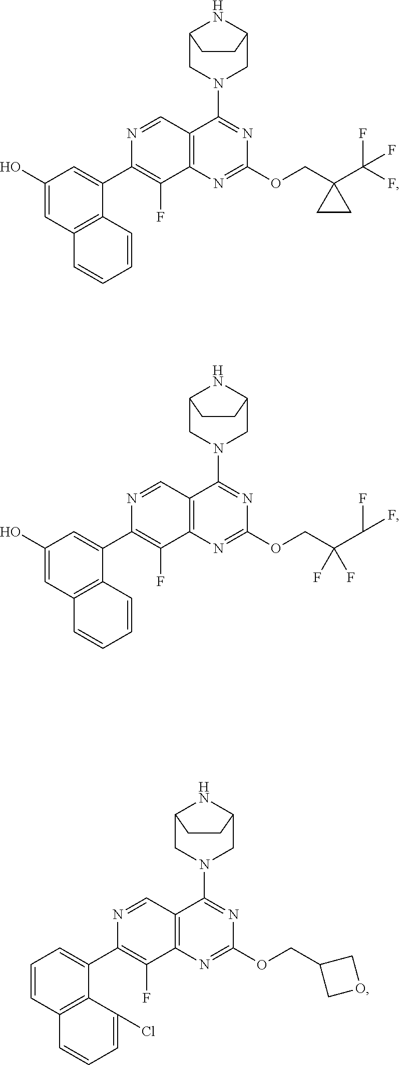 [SMILES: Oc1cc(-c2ncc3c(N4CC5CCC(C4)N5)nc(OCC4(C(F)(F)F)CC4)nc3c2F)c2ccccc2c1 ; Oc1cc(-c2ncc3c(N4CC5CCC(C4)N5)nc(OCC(F)(F)C(F)F)nc3c2F)c2ccccc2c1 ; Fc1c(-c2cccc3cccc(Cl)c23)ncc2c(N3CC4CCC(C3)N4)nc(OCC3COC3)nc12]

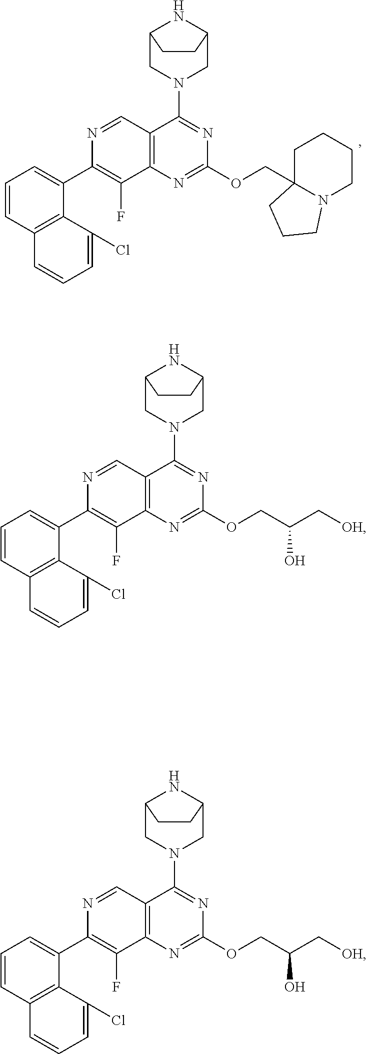 [SMILES: Fc1c(-c2cccc3cccc(Cl)c23)ncc2c(N3CC4CCC(C3)N4)nc(OCC34CCCCN3CCC4)nc12 ; OC[C@H](O)COc1nc(N2CC3CCC(C2)N3)c2cnc(-c3cccc4cccc(Cl)c34)c(F)c2n1 ; OC[C@@H](O)COc1nc(N2CC3CCC(C2)N3)c2cnc(-c3cccc4cccc(Cl)c34)c(F)c2n1]

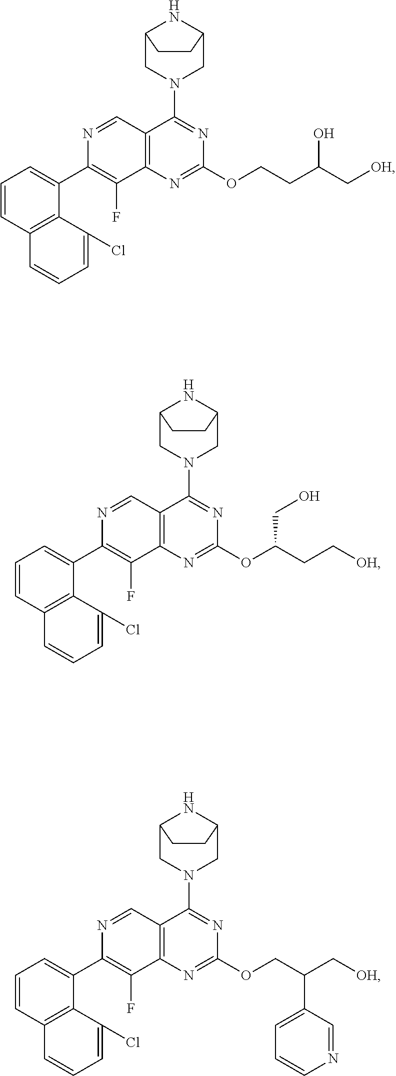 [SMILES: OCC(O)CCOc1nc(N2CC3CCC(C2)N3)c2cnc(-c3cccc4cccc(Cl)c34)c(F)c2n1 ; OCC[C@@H](CO)Oc1nc(N2CC3CCC(C2)N3)c2cnc(-c3cccc4cccc(Cl)c34)c(F)c2n1 ; OCC(COc1nc(N2CC3CCC(C2)N3)c2cnc(-c3cccc4cccc(Cl)c34)c(F)c2n1)c1cccnc1]

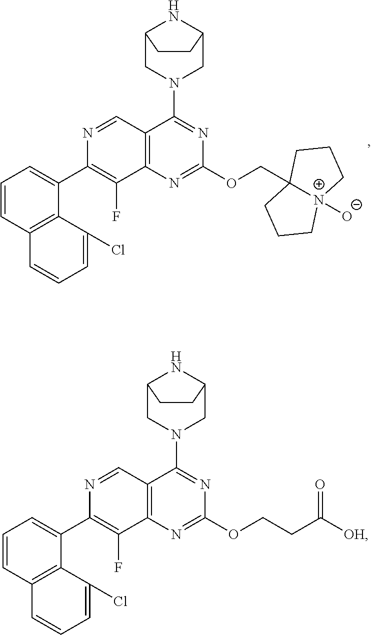 [SMILES: [O-][N+]12CCCC1(COc1nc(N3CC4CCC(C3)N4)c3cnc(-c4cccc5cccc(Cl)c45)c(F)c3n1)CCC2 ; O=C(O)CCOc1nc(N2CC3CCC(C2)N3)c2cnc(-c3cccc4cccc(Cl)c34)c(F)c2n1]

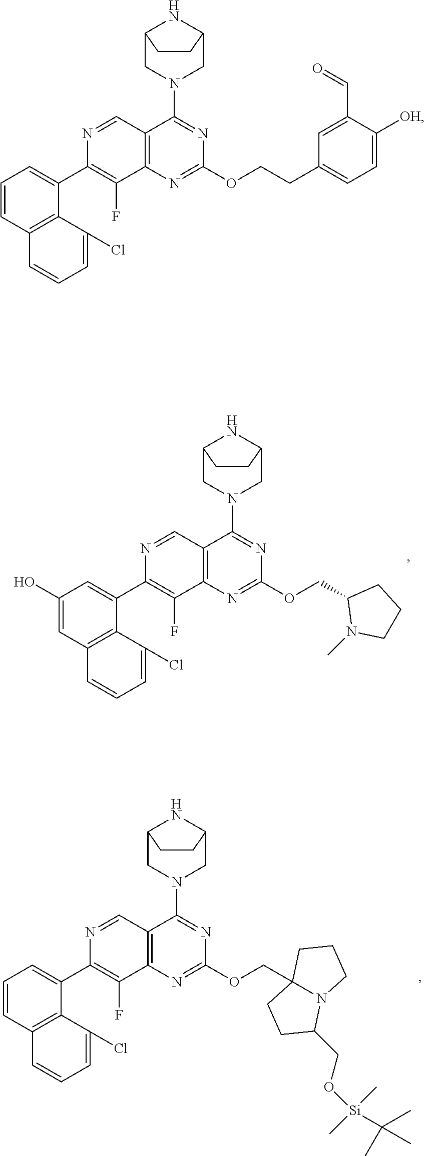 [SMILES: O=Cc1cc(CCOc2nc(N3CC4CCC(C3)N4)c3cnc(-c4cccc5cccc(Cl)c45)c(F)c3n2)ccc1O ; CN1CCC[C@H]1COc1nc(N2CC3CCC(C2)N3)c2cnc(-c3cc(O)cc4cccc(Cl)c34)c(F)c2n1 ; CC(C)(C)[Si](C)(C)OCC1CCC2(COc3nc(N4CC5CCC(C4)N5)c4cnc(-c5cccc6cccc(Cl)c56)c(F)c4n3)CCCN12]

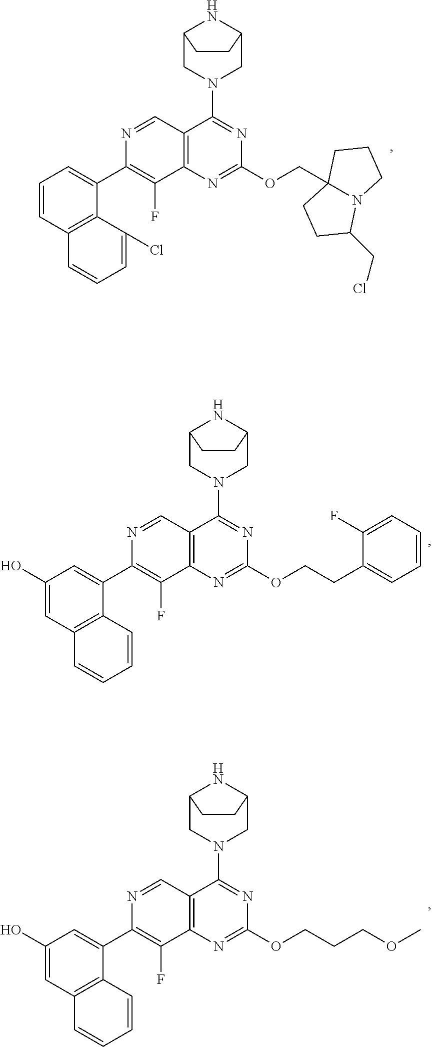 [SMILES: Fc1c(-c2cccc3cccc(Cl)c23)ncc2c(N3CC4CCC(C3)N4)nc(OCC34CCCN3C(CCl)CC4)nc12 ; Oc1cc(-c2ncc3c(N4CC5CCC(C4)N5)nc(OCCc4ccccc4F)nc3c2F)c2ccccc2c1 ; COCCCOc1nc(N2CC3CCC(C2)N3)c2cnc(-c3cc(O)cc4ccccc34)c(F)c2n1]

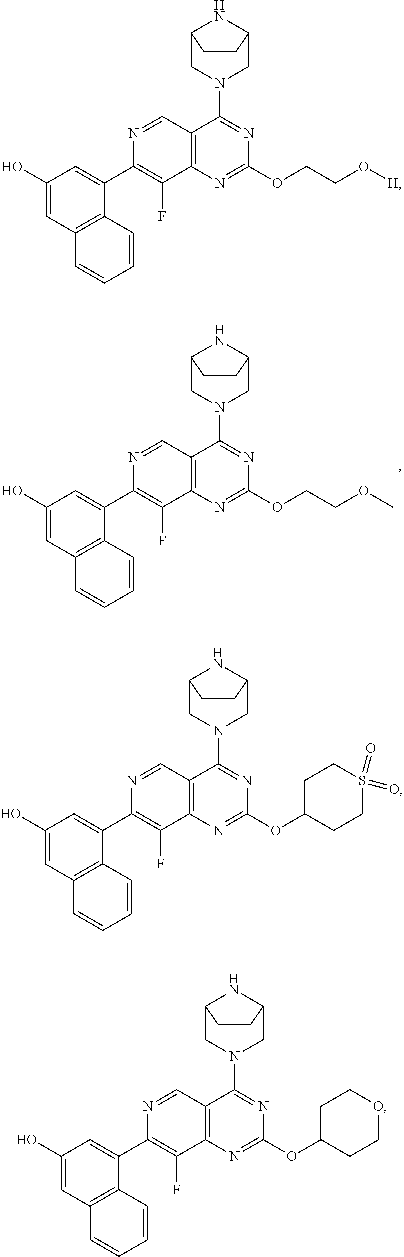

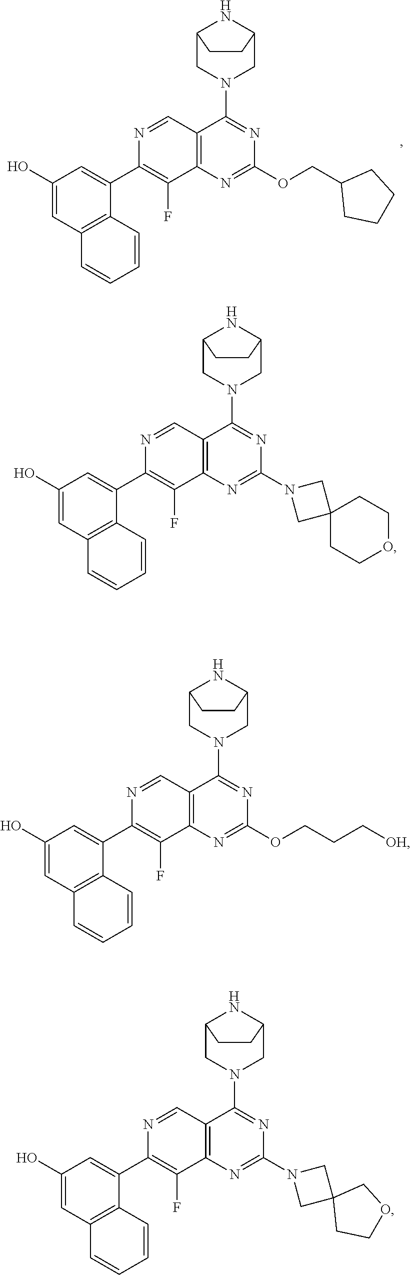 [SMILES: Oc1cc(-c2ncc3c(N4CC5CCC(C4)N5)nc(OCC4CCCC4)nc3c2F)c2ccccc2c1 ; Oc1cc(-c2ncc3c(N4CC5CCC(C4)N5)nc(N4CC5(CCOCC5)C4)nc3c2F)c2ccccc2c1 ; OCCCOc1nc(N2CC3CCC(C2)N3)c2cnc(-c3cc(O)cc4ccccc34)c(F)c2n1 ; Oc1cc(-c2ncc3c(N4CC5CCC(C4)N5)nc(N4CC5(CCOC5)C4)nc3c2F)c2ccccc2c1]

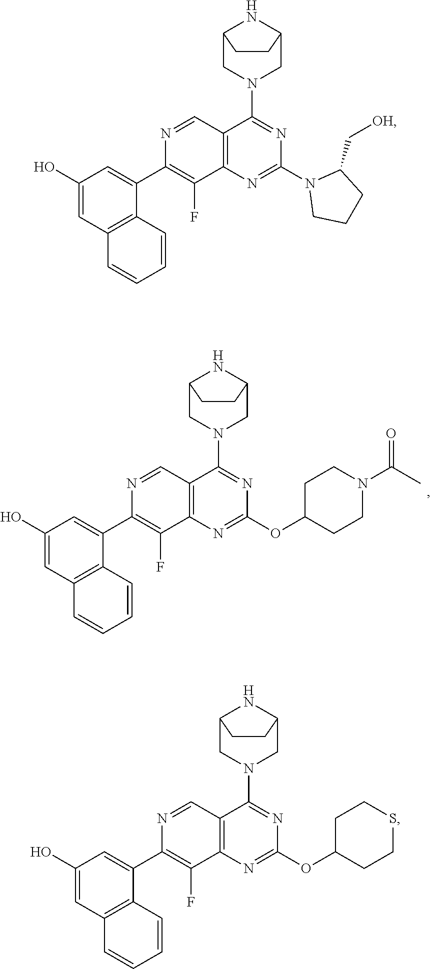 [SMILES: OC[C@@H]1CCCN1c1nc(N2CC3CCC(C2)N3)c2cnc(-c3cc(O)cc4ccccc34)c(F)c2n1 ; CC(=O)N1CCC(Oc2nc(N3CC4CCC(C3)N4)c3cnc(-c4cc(O)cc5ccccc45)c(F)c3n2)CC1 ; Oc1cc(-c2ncc3c(N4CC5CCC(C4)N5)nc(OC4CCSCC4)nc3c2F)c2ccccc2c1]

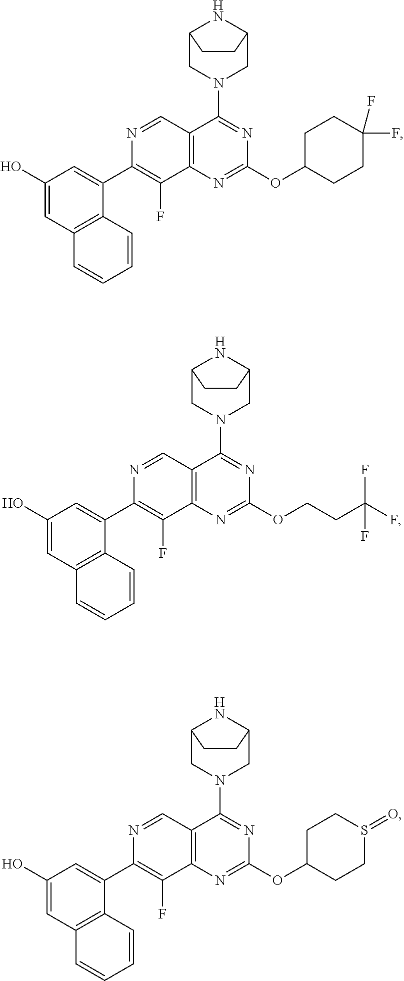 [SMILES: Oc1cc(-c2ncc3c(N4CC5CCC(C4)N5)nc(OC4CCC(F)(F)CC4)nc3c2F)c2ccccc2c1 ; Oc1cc(-c2ncc3c(N4CC5CCC(C4)N5)nc(OCCC(F)(F)F)nc3c2F)c2ccccc2c1 ; O=S1CCC(Oc2nc(N3CC4CCC(C3)N4)c3cnc(-c4cc(O)cc5ccccc45)c(F)c3n2)CC1]

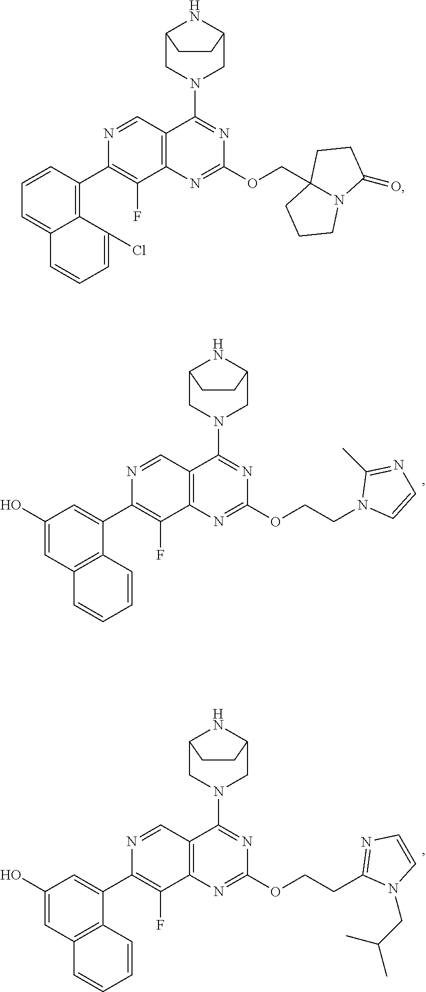 [SMILES: O=C1CCC2(COc3nc(N4CC5CCC(C4)N5)c4cnc(-c5cccc6cccc(Cl)c56)c(F)c4n3)CCCN12 ; Cc1nccn1CCOc1nc(N2CC3CCC(C2)N3)c2cnc(-c3cc(O)cc4ccccc34)c(F)c2n1 ; CC(C)Cn1ccnc1CCOc1nc(N2CC3CCC(C2)N3)c2cnc(-c3cc(O)cc4ccccc34)c(F)c2n1]

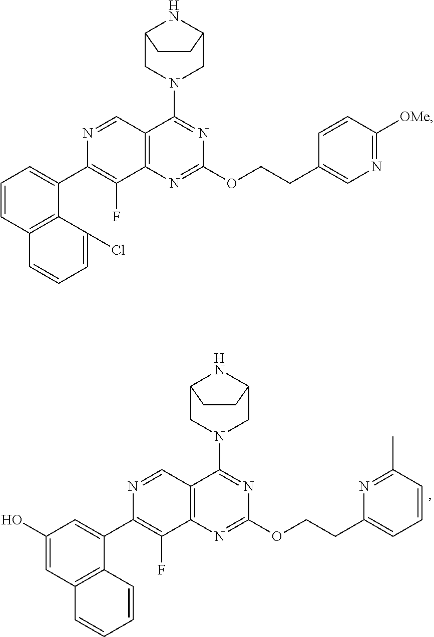 [SMILES: COc1ccc(CCOc2nc(N3CC4CCC(C3)N4)c3cnc(-c4cccc5cccc(Cl)c45)c(F)c3n2)cn1 ; Cc1cccc(CCOc2nc(N3CC4CCC(C3)N4)c3cnc(-c4cc(O)cc5ccccc45)c(F)c3n2)n1]

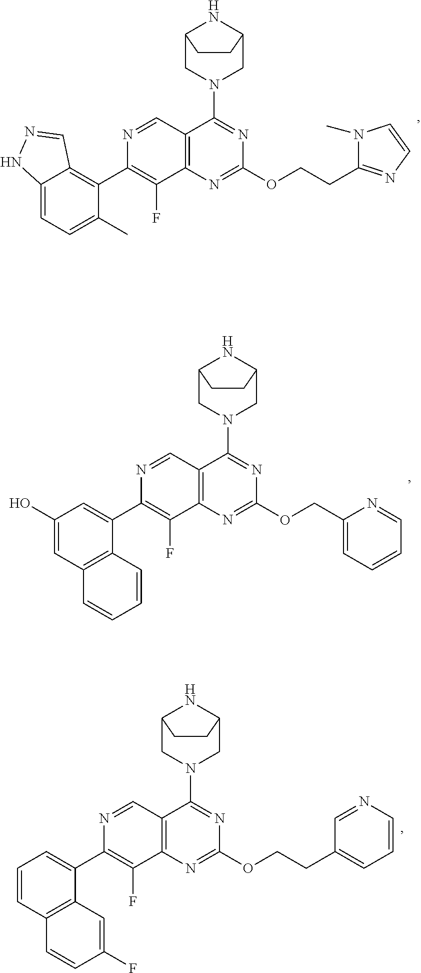 [SMILES: Cc1ccc2[nH]ncc2c1-c1ncc2c(N3CC4CCC(C3)N4)nc(OCCc3nccn3C)nc2c1F ; Oc1cc(-c2ncc3c(N4CC5CCC(C4)N5)nc(OCc4ccccn4)nc3c2F)c2ccccc2c1 ; Fc1ccc2cccc(-c3ncc4c(N5CC6CCC(C5)N6)nc(OCCc5cccnc5)nc4c3F)c2c1]

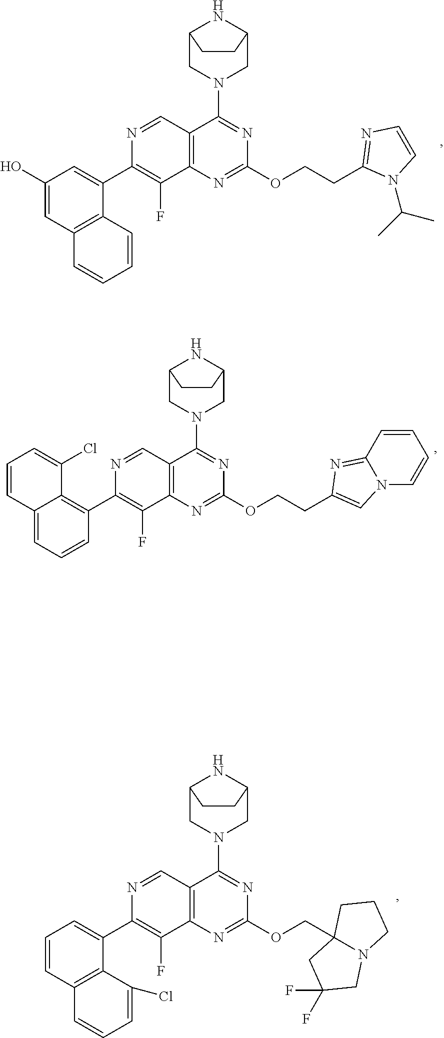 [SMILES: CC(C)n1ccnc1CCOc1nc(N2CC3CCC(C2)N3)c2cnc(-c3cc(O)cc4ccccc34)c(F)c2n1 ; Fc1c(-c2cccc3cccc(Cl)c23)ncc2c(N3CC4CCC(C3)N4)nc(OCCc3cn4ccccc4n3)nc12 ; Fc1c(-c2cccc3cccc(Cl)c23)ncc2c(N3CC4CCC(C3)N4)nc(OCC34CCCN3CC(F)(F)C4)nc12]

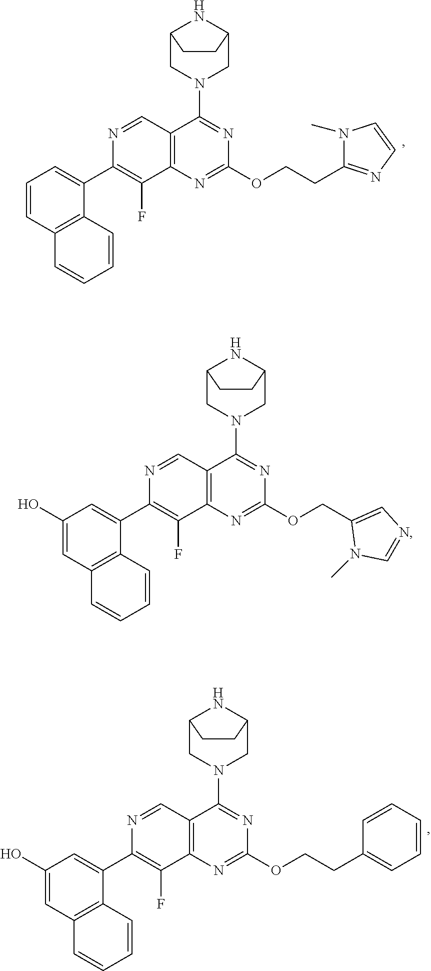 [SMILES: Cn1ccnc1CCOc1nc(N2CC3CCC(C2)N3)c2cnc(-c3cccc4ccccc34)c(F)c2n1 ; Cn1cncc1COc1nc(N2CC3CCC(C2)N3)c2cnc(-c3cc(O)cc4ccccc34)c(F)c2n1 ; Oc1cc(-c2ncc3c(N4CC5CCC(C4)N5)nc(OCCc4ccccc4)nc3c2F)c2ccccc2c1]

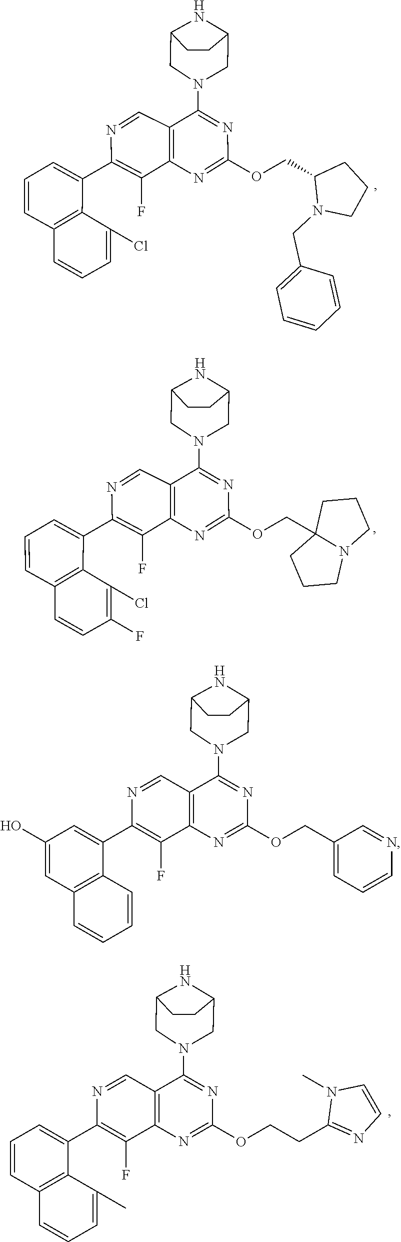 [SMILES: Fc1c(-c2cccc3cccc(Cl)c23)ncc2c(N3CC4CCC(C3)N4)nc(OC[C@@H]3CCCN3Cc3ccccc3)nc12 ; Fc1ccc2cccc(-c3ncc4c(N5CC6CCC(C5)N6)nc(OCC56CCCN5CCC6)nc4c3F)c2c1Cl ; Oc1cc(-c2ncc3c(N4CC5CCC(C4)N5)nc(OCc4cccnc4)nc3c2F)c2ccccc2c1 ; Cc1cccc2cccc(-c3ncc4c(N5CC6CCC(C5)N6)nc(OCCc5nccn5C)nc4c3F)c12]

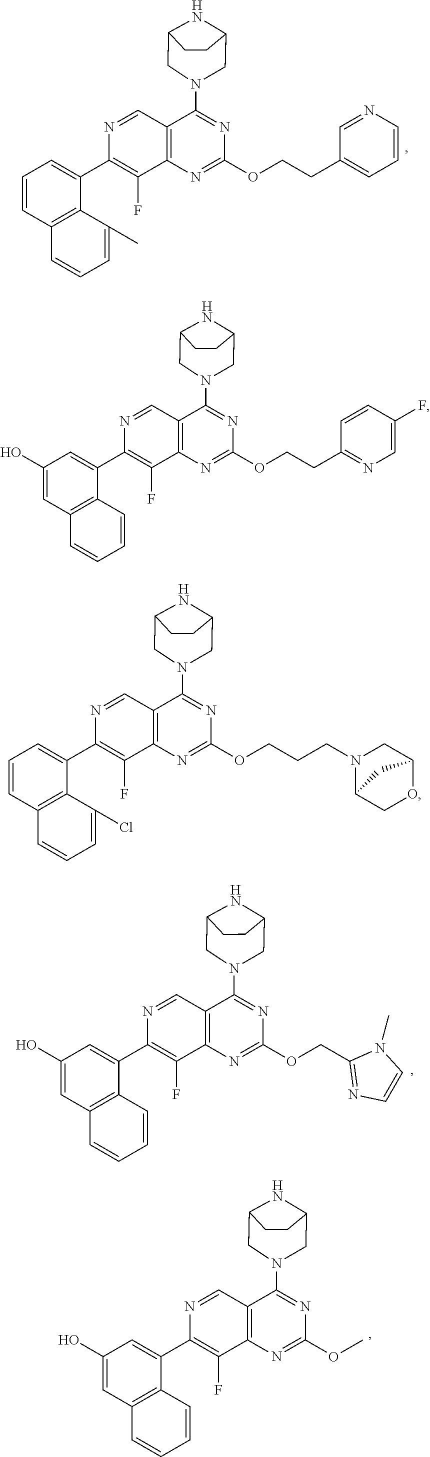 [SMILES: Cc1cccc2cccc(-c3ncc4c(N5CC6CCC(C5)N6)nc(OCCc5cccnc5)nc4c3F)c12 ; Oc1cc(-c2ncc3c(N4CC5CCC(C4)N5)nc(OCCc4ccc(F)cn4)nc3c2F)c2ccccc2c1 ; Fc1c(-c2cccc3cccc(Cl)c23)ncc2c(N3CC4CCC(C3)N4)nc(OCCCN3C[C@@H]4C[C@H]3CO4)nc12 ; Cn1ccnc1COc1nc(N2CC3CCC(C2)N3)c2cnc(-c3cc(O)cc4ccccc34)c(F)c2n1 ; COc1nc(N2CC3CCC(C2)N3)c2cnc(-c3cc(O)cc4ccccc34)c(F)c2n1]

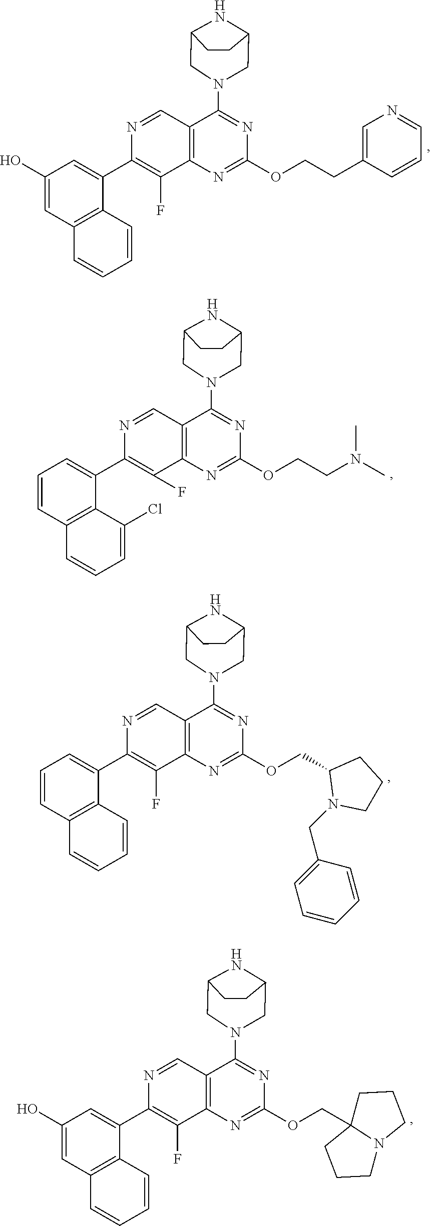 [SMILES: Oc1cc(-c2ncc3c(N4CC5CCC(C4)N5)nc(OCCc4cccnc4)nc3c2F)c2ccccc2c1 ; CN(C)CCOc1nc(N2CC3CCC(C2)N3)c2cnc(-c3cccc4cccc(Cl)c34)c(F)c2n1 ; Fc1c(-c2cccc3ccccc23)ncc2c(N3CC4CCC(C3)N4)nc(OC[C@@H]3CCCN3Cc3ccccc3)nc12 ; Oc1cc(-c2ncc3c(N4CC5CCC(C4)N5)nc(OCC45CCCN4CCC5)nc3c2F)c2ccccc2c1]

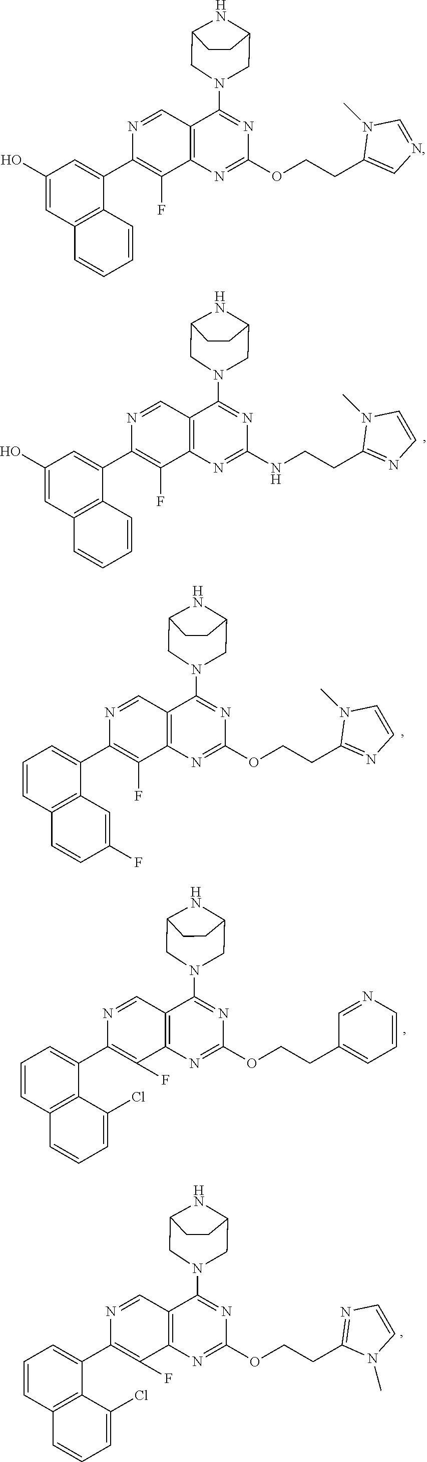 [SMILES: Cn1cncc1CCOc1nc(N2CC3CCC(C2)N3)c2cnc(-c3cc(O)cc4ccccc34)c(F)c2n1 ; Cn1ccnc1CCNc1nc(N2CC3CCC(C2)N3)c2cnc(-c3cc(O)cc4ccccc34)c(F)c2n1 ; Cn1ccnc1CCOc1nc(N2CC3CCC(C2)N3)c2cnc(-c3cccc4ccc(F)cc34)c(F)c2n1 ; Fc1c(-c2cccc3cccc(Cl)c23)ncc2c(N3CC4CCC(C3)N4)nc(OCCc3cccnc3)nc12 ; Cn1ccnc1CCOc1nc(N2CC3CCC(C2)N3)c2cnc(-c3cccc4cccc(Cl)c34)c(F)c2n1]

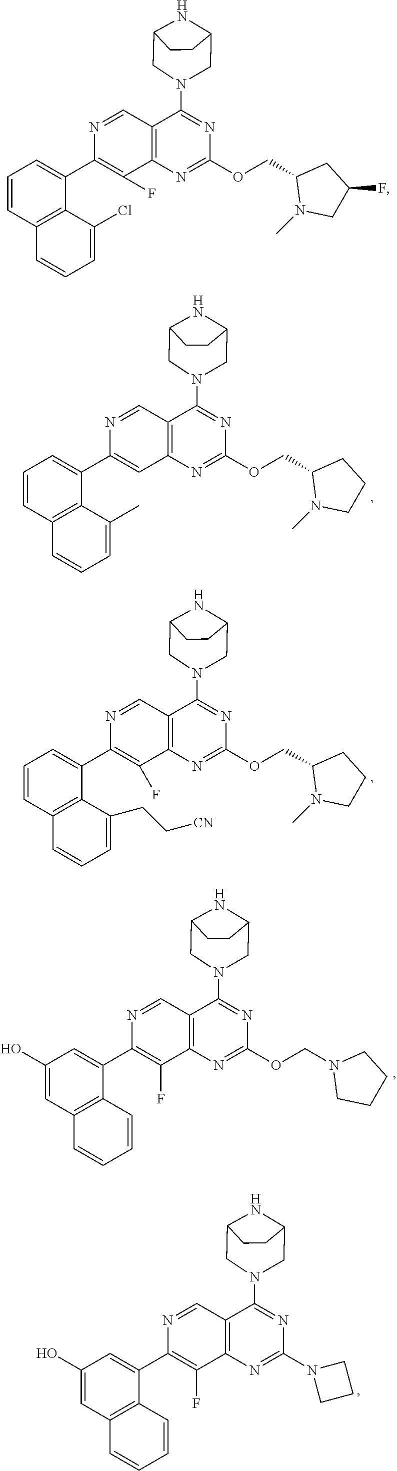 [SMILES: CN1C[C@H](F)C[C@H]1COc1nc(N2CC3CCC(C2)N3)c2cnc(-c3cccc4cccc(Cl)c34)c(F)c2n1 ; Cc1cccc2cccc(-c3cc4nc(OC[C@@H]5CCCN5C)nc(N5CC6CCC(C5)N6)c4cn3)c12 ; CN1CCC[C@H]1COc1nc(N2CC3CCC(C2)N3)c2cnc(-c3cccc4cccc(CCC#N)c34)c(F)c2n1 ; Oc1cc(-c2ncc3c(N4CC5CCC(C4)N5)nc(OCN4CCCC4)nc3c2F)c2ccccc2c1 ; Oc1cc(-c2ncc3c(N4CC5CCC(C4)N5)nc(N4CCC4)nc3c2F)c2ccccc2c1]

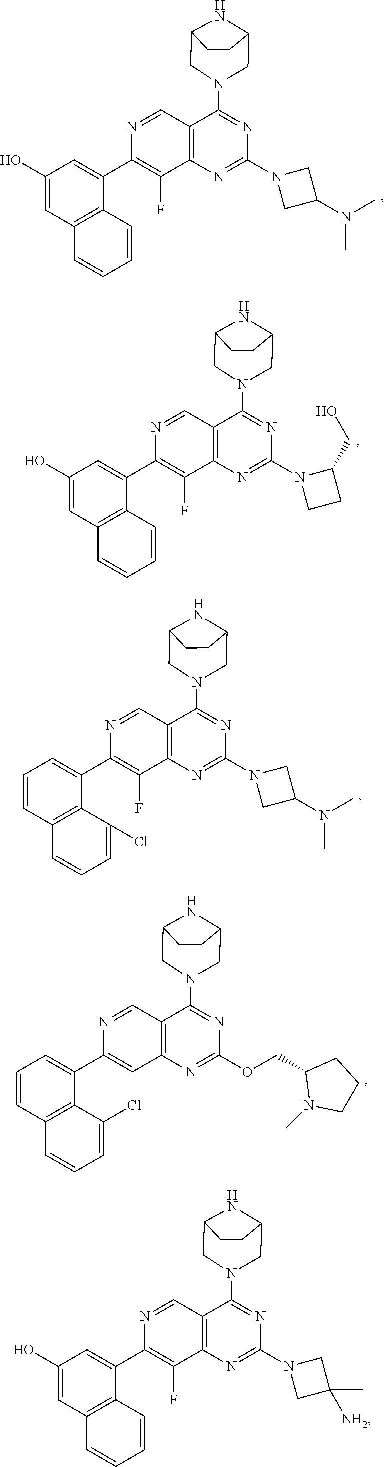 [SMILES: CN(C)C1CN(c2nc(N3CC4CCC(C3)N4)c3cnc(-c4cc(O)cc5ccccc45)c(F)c3n2)C1 ; OC[C@@H]1CCN1c1nc(N2CC3CCC(C2)N3)c2cnc(-c3cc(O)cc4ccccc34)c(F)c2n1 ; CN(C)C1CN(c2nc(N3CC4CCC(C3)N4)c3cnc(-c4cccc5cccc(Cl)c45)c(F)c3n2)C1 ; CN1CCC[C@H]1COc1nc(N2CC3CCC(C2)N3)c2cnc(-c3cccc4cccc(Cl)c34)cc2n1 ; CC1(N)CN(c2nc(N3CC4CCC(C3)N4)c3cnc(-c4cc(O)cc5ccccc45)c(F)c3n2)C1]

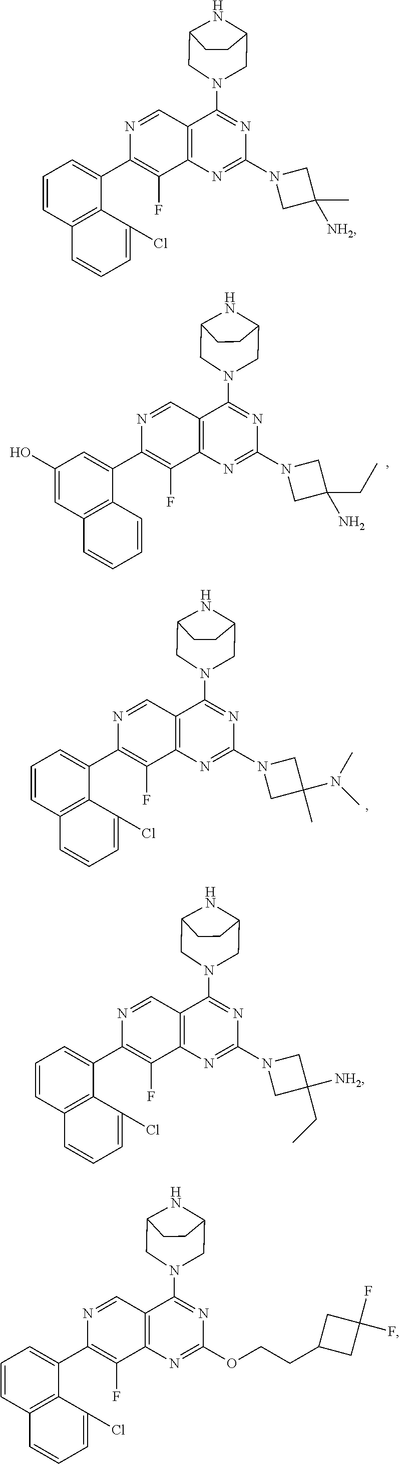 [SMILES: CC1(N)CN(c2nc(N3CC4CCC(C3)N4)c3cnc(-c4cccc5cccc(Cl)c45)c(F)c3n2)C1 ; CCC1(N)CN(c2nc(N3CC4CCC(C3)N4)c3cnc(-c4cc(O)cc5ccccc45)c(F)c3n2)C1 ; CN(C)C1(C)CN(c2nc(N3CC4CCC(C3)N4)c3cnc(-c4cccc5cccc(Cl)c45)c(F)c3n2)C1 ; CCC1(N)CN(c2nc(N3CC4CCC(C3)N4)c3cnc(-c4cccc5cccc(Cl)c45)c(F)c3n2)C1 ; Fc1c(-c2cccc3cccc(Cl)c23)ncc2c(N3CC4CCC(C3)N4)nc(OCCC3CC(F)(F)C3)nc12]

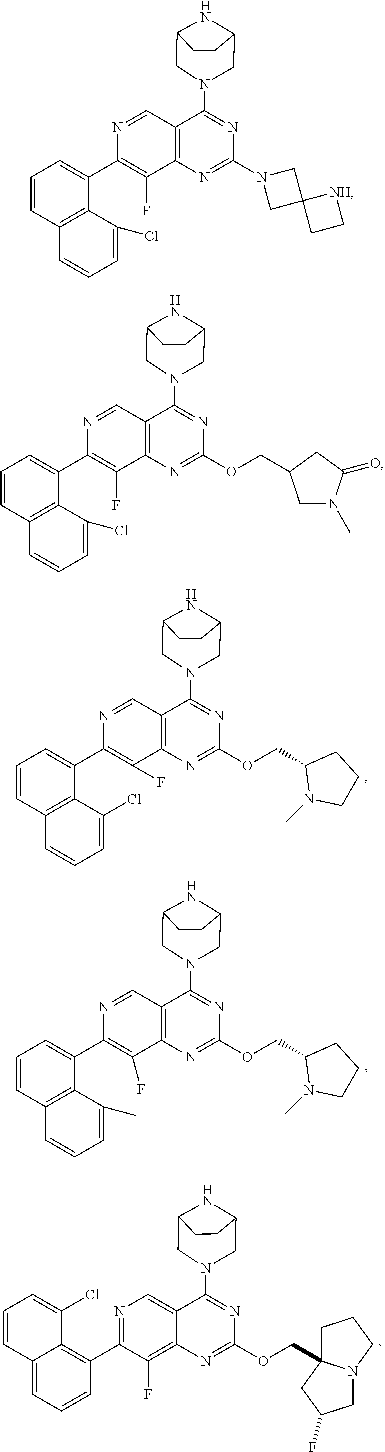

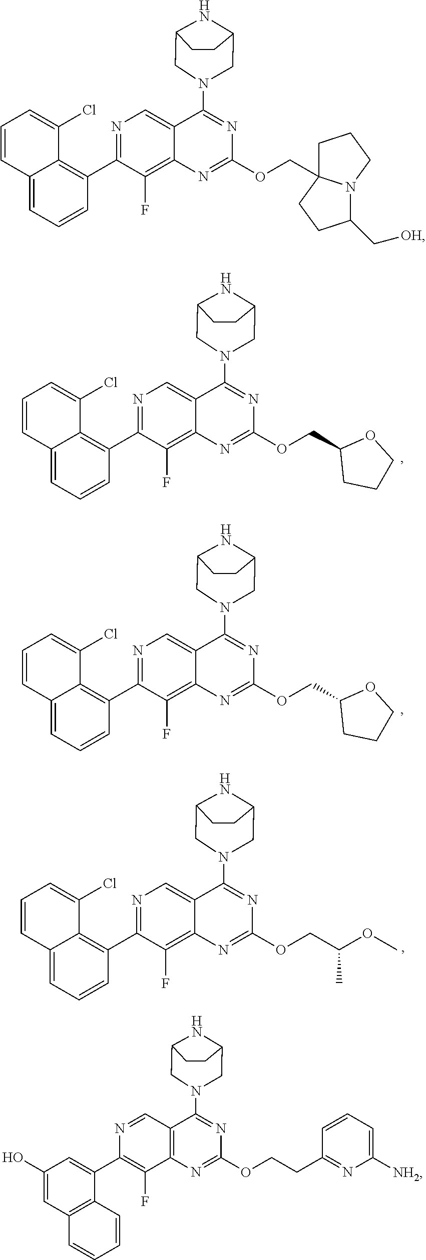 [SMILES: OCC1CCC2(COc3nc(N4CC5CCC(C4)N5)c4cnc(-c5cccc6cccc(Cl)c56)c(F)c4n3)CCCN12 ; Fc1c(-c2cccc3cccc(Cl)c23)ncc2c(N3CC4CCC(C3)N4)nc(OC[C@@H]3CCCO3)nc12 ; Fc1c(-c2cccc3cccc(Cl)c23)ncc2c(N3CC4CCC(C3)N4)nc(OC[C@H]3CCCO3)nc12 ; CO[C@H](C)COc1nc(N2CC3CCC(C2)N3)c2cnc(-c3cccc4cccc(Cl)c34)c(F)c2n1 ; Nc1cccc(CCOc2nc(N3CC4CCC(C3)N4)c3cnc(-c4cc(O)cc5ccccc45)c(F)c3n2)n1]

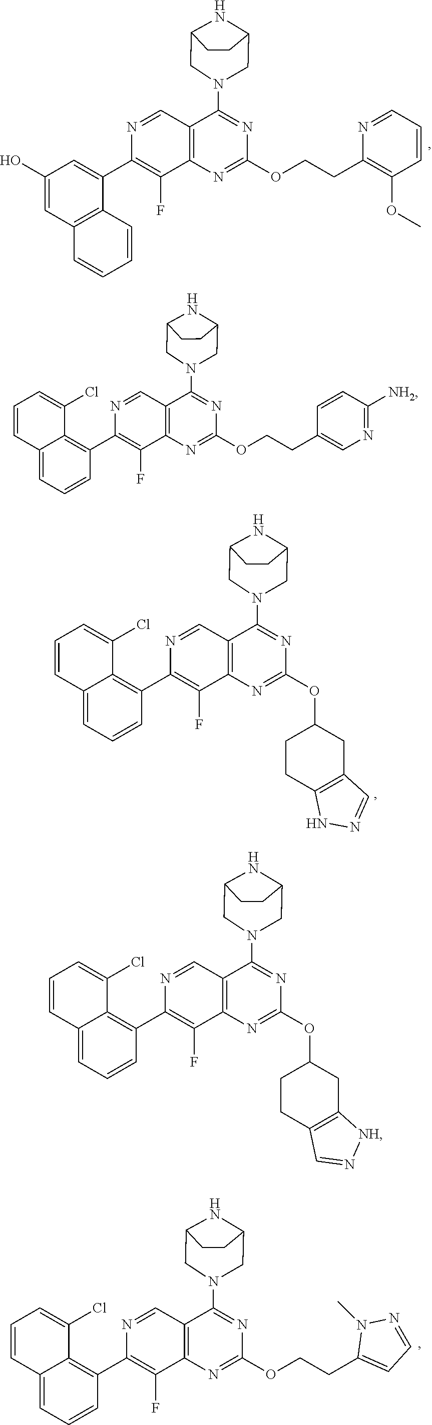 [SMILES: COc1cccnc1CCOc1nc(N2CC3CCC(C2)N3)c2cnc(-c3cc(O)cc4ccccc34)c(F)c2n1 ; Nc1ccc(CCOc2nc(N3CC4CCC(C3)N4)c3cnc(-c4cccc5cccc(Cl)c45)c(F)c3n2)cn1 ; Fc1c(-c2cccc3cccc(Cl)c23)ncc2c(N3CC4CCC(C3)N4)nc(OC3CCc4[nH]ncc4C3)nc12 ; Fc1c(-c2cccc3cccc(Cl)c23)ncc2c(N3CC4CCC(C3)N4)nc(OC3CCc4cn[nH]c4C3)nc12 ; Cn1nccc1CCOc1nc(N2CC3CCC(C2)N3)c2cnc(-c3cccc4cccc(Cl)c34)c(F)c2n1]

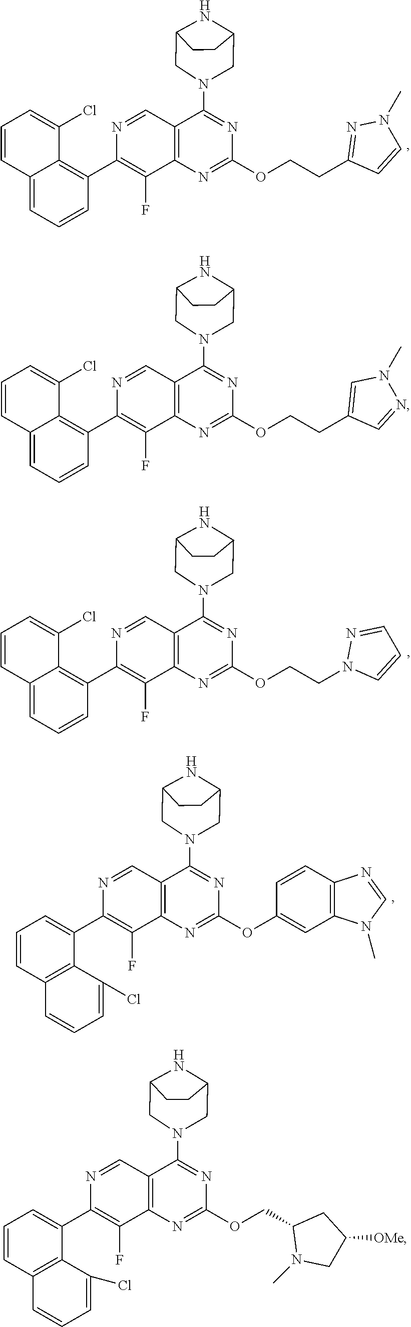 [SMILES: Cn1ccc(CCOc2nc(N3CC4CCC(C3)N4)c3cnc(-c4cccc5cccc(Cl)c45)c(F)c3n2)n1 ; Cn1cc(CCOc2nc(N3CC4CCC(C3)N4)c3cnc(-c4cccc5cccc(Cl)c45)c(F)c3n2)cn1 ; Fc1c(-c2cccc3cccc(Cl)c23)ncc2c(N3CC4CCC(C3)N4)nc(OCCn3cccn3)nc12 ; Cn1cnc2ccc(Oc3nc(N4CC5CCC(C4)N5)c4cnc(-c5cccc6cccc(Cl)c56)c(F)c4n3)cc21 ; CO[C@H]1C[C@@H](COc2nc(N3CC4CCC(C3)N4)c3cnc(-c4cccc5cccc(Cl)c45)c(F)c3n2)N(C)C1]

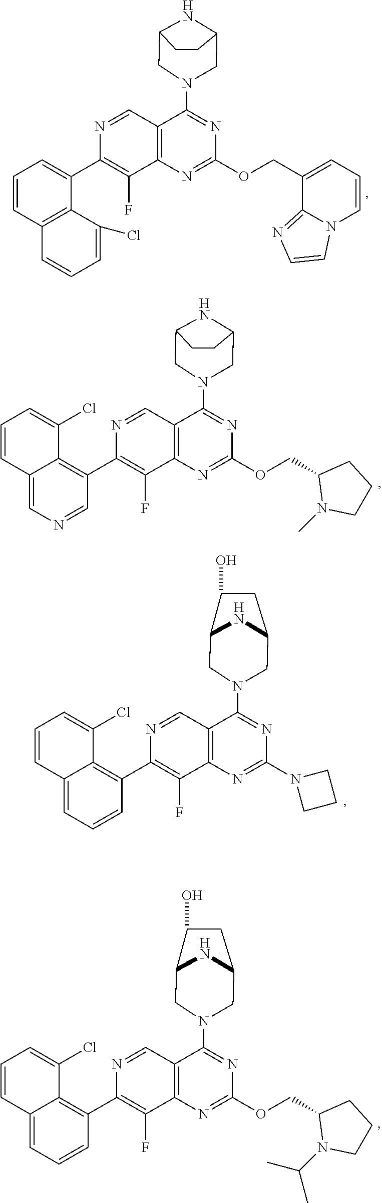 [SMILES: Fc1c(-c2cccc3cccc(Cl)c23)ncc2c(N3CC4CCC(C3)N4)nc(OCc3cccn4ccnc34)nc12 ; CN1CCC[C@H]1COc1nc(N2CC3CCC(C2)N3)c2cnc(-c3cncc4cccc(Cl)c34)c(F)c2n1 ; O[C@@H]1C[C@@H]2CN(c3nc(N4CCC4)nc4c(F)c(-c5cccc6cccc(Cl)c56)ncc34)C[C@H]1N2 ; CC(C)N1CCC[C@H]1COc1nc(N2C[C@H]3C[C@@H](O)[C@@H](C2)N3)c2cnc(-c3cccc4cccc(Cl)c34)c(F)c2n1]

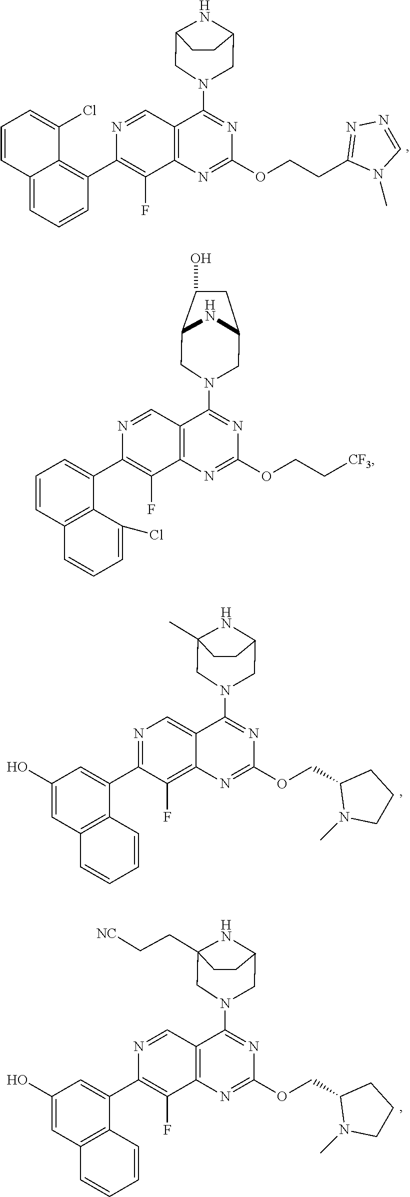 [SMILES: Cn1cnnc1CCOc1nc(N2CC3CCC(C2)N3)c2cnc(-c3cccc4cccc(Cl)c34)c(F)c2n1 ; O[C@@H]1C[C@@H]2CN(c3nc(OCCC(F)(F)F)nc4c(F)c(-c5cccc6cccc(Cl)c56)ncc34)C[C@H]1N2 ; CN1CCC[C@H]1COc1nc(N2CC3CCC(C)(C2)N3)c2cnc(-c3cc(O)cc4ccccc34)c(F)c2n1 ; CN1CCC[C@H]1COc1nc(N2CC3CCC(CCC#N)(C2)N3)c2cnc(-c3cc(O)cc4ccccc34)c(F)c2n1]

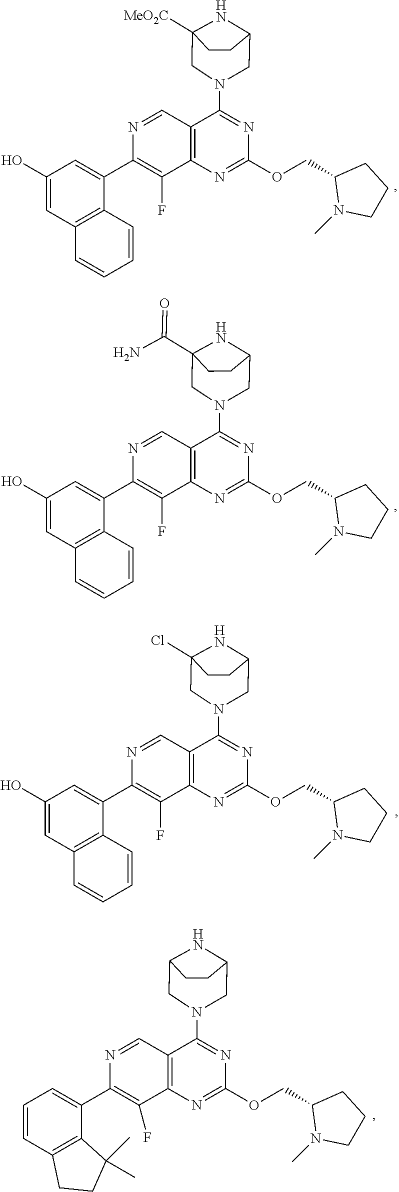 [SMILES: COC(=O)C12CCC(CN(c3nc(OC[C@@H]4CCCN4C)nc4c(F)c(-c5cc(O)cc6ccccc56)ncc34)C1)N2 ; CN1CCC[C@H]1COc1nc(N2CC3CCC(C(N)=O)(C2)N3)c2cnc(-c3cc(O)cc4ccccc34)c(F)c2n1 ; CN1CCC[C@H]1COc1nc(N2CC3CCC(Cl)(C2)N3)c2cnc(-c3cc(O)cc4ccccc34)c(F)c2n1 ; CN1CCC[C@H]1COc1nc(N2CC3CCC(C2)N3)c2cnc(-c3cccc4c3C(C)(C)CC4)c(F)c2n1]

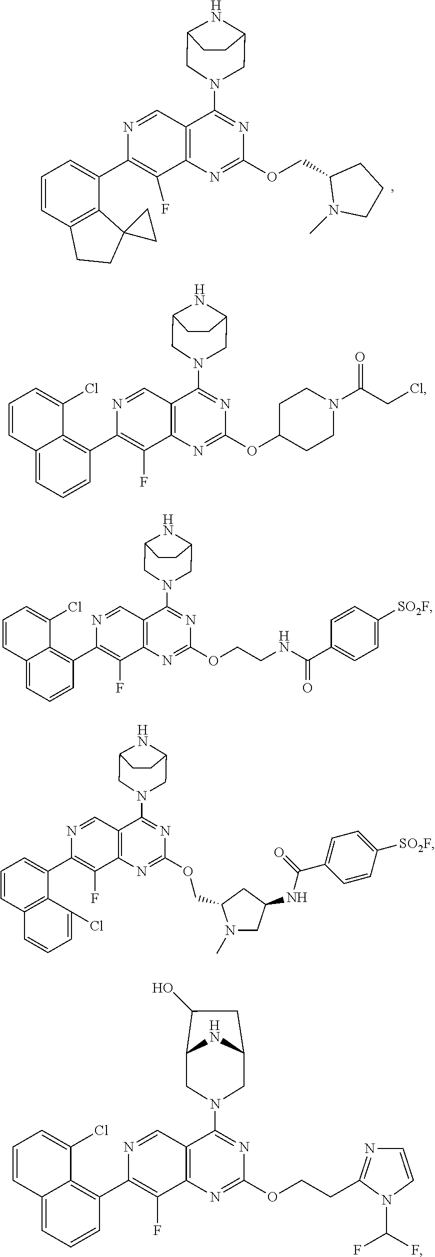 [SMILES: CN1CCC[C@H]1COc1nc(N2CC3CCC(C2)N3)c2cnc(-c3cccc4c3C3(CC4)CC3)c(F)c2n1 ; O=C(CCl)N1CCC(Oc2nc(N3CC4CCC(C3)N4)c3cnc(-c4cccc5cccc(Cl)c45)c(F)c3n2)CC1 ; O=C(NCCOc1nc(N2CC3CCC(C2)N3)c2cnc(-c3cccc4cccc(Cl)c34)c(F)c2n1)c1ccc(S(=O)(=O)F)cc1 ; CN1C[C@H](NC(=O)c2ccc(S(=O)(=O)F)cc2)C[C@H]1COc1nc(N2CC3CCC(C2)N3)c2cnc(-c3cccc4cccc(Cl)c34)c(F)c2n1 ; OC1C[C@@H]2CN(c3nc(OCCc4nccn4C(F)F)nc4c(F)c(-c5cccc6cccc(Cl)c56)ncc34)C[C@H]1N2]

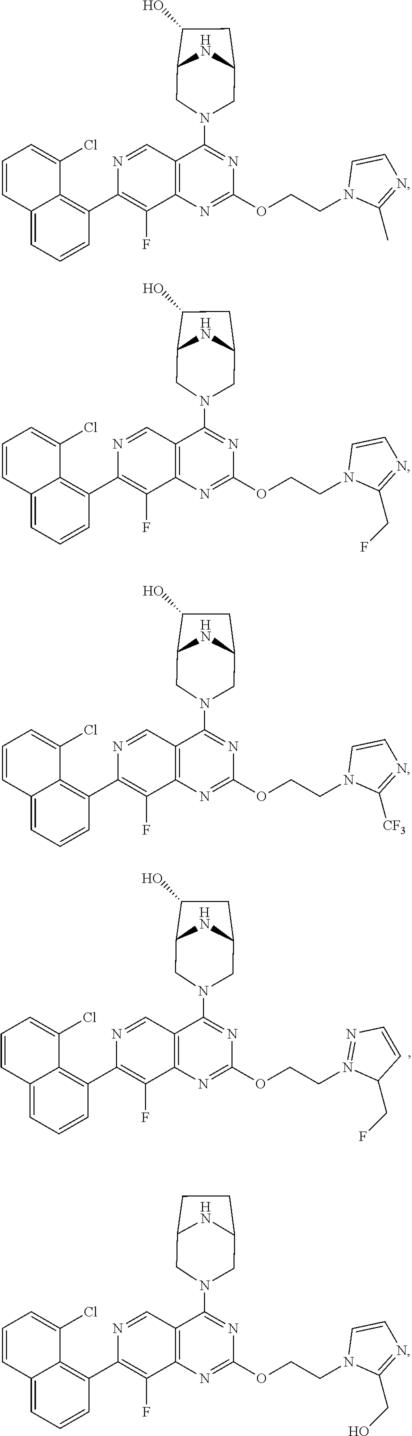

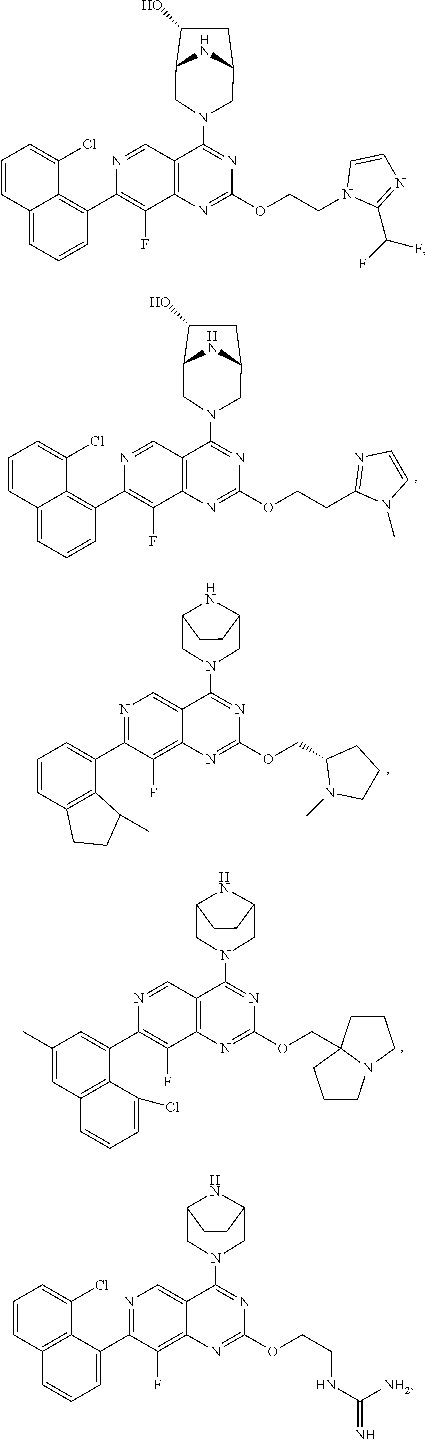 [SMILES: O[C@@H]1C[C@@H]2CN(c3nc(OCCn4ccnc4C(F)F)nc4c(F)c(-c5cccc6cccc(Cl)c56)ncc34)C[C@H]1N2 ; Cn1ccnc1CCOc1nc(N2C[C@H]3C[C@@H](O)[C@@H](C2)N3)c2cnc(-c3cccc4cccc(Cl)c34)c(F)c2n1 ; CC1CCc2cccc(-c3ncc4c(N5CC6CCC(C5)N6)nc(OC[C@@H]5CCCN5C)nc4c3F)c21 ; Cc1cc(-c2ncc3c(N4CC5CCC(C4)N5)nc(OCC45CCCN4CCC5)nc3c2F)c2c(Cl)cccc2c1 ; N=C(N)NCCOc1nc(N2CC3CCC(C2)N3)c2cnc(-c3cccc4cccc(Cl)c34)c(F)c2n1]

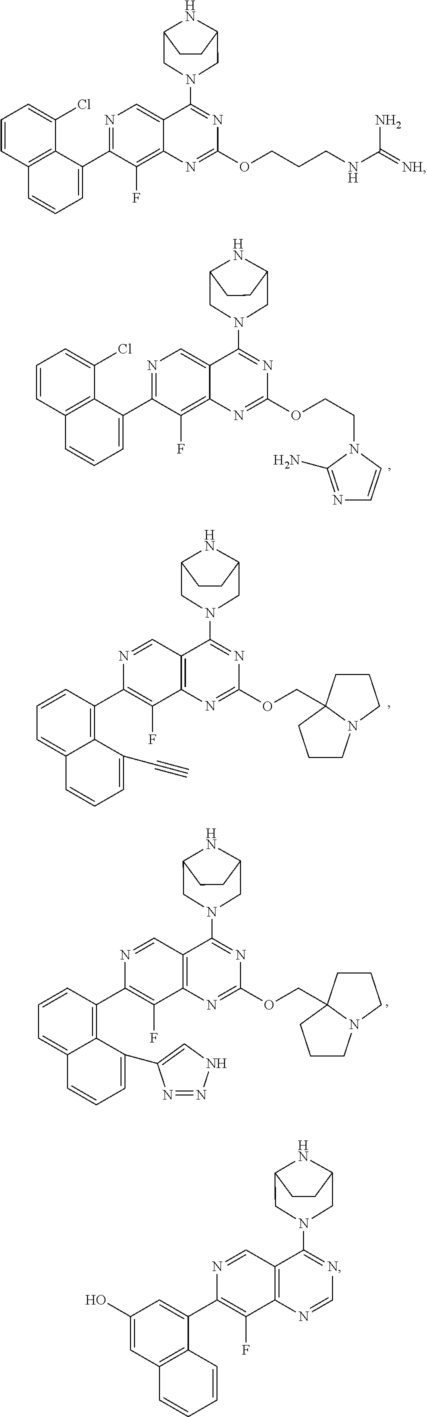 [SMILES: N=C(N)NCCCOc1nc(N2CC3CCC(C2)N3)c2cnc(-c3cccc4cccc(Cl)c34)c(F)c2n1 ; Nc1nccn1CCOc1nc(N2CC3CCC(C2)N3)c2cnc(-c3cccc4cccc(Cl)c34)c(F)c2n1 ; C#Cc1cccc2cccc(-c3ncc4c(N5CC6CCC(C5)N6)nc(OCC56CCCN5CCC6)nc4c3F)c12 ; Fc1c(-c2cccc3cccc(-c4c[nH]nn4)c23)ncc2c(N3CC4CCC(C3)N4)nc(OCC34CCCN3CCC4)nc12 ; Oc1cc(-c2ncc3c(N4CC5CCC(C4)N5)ncnc3c2F)c2ccccc2c1]

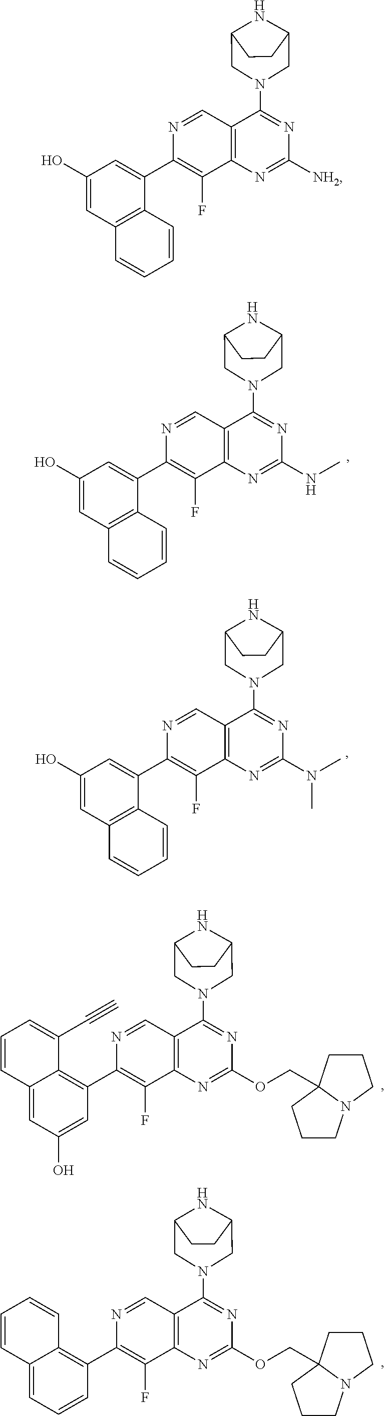 [SMILES: Nc1nc(N2CC3CCC(C2)N3)c2cnc(-c3cc(O)cc4ccccc34)c(F)c2n1 ; CNc1nc(N2CC3CCC(C2)N3)c2cnc(-c3cc(O)cc4ccccc34)c(F)c2n1 ; CN(C)c1nc(N2CC3CCC(C2)N3)c2cnc(-c3cc(O)cc4ccccc34)c(F)c2n1 ; C#Cc1cccc2cc(O)cc(-c3ncc4c(N5CC6CCC(C5)N6)nc(OCC56CCCN5CCC6)nc4c3F)c12 ; Fc1c(-c2cccc3ccccc23)ncc2c(N3CC4CCC(C3)N4)nc(OCC34CCCN3CCC4)nc12]

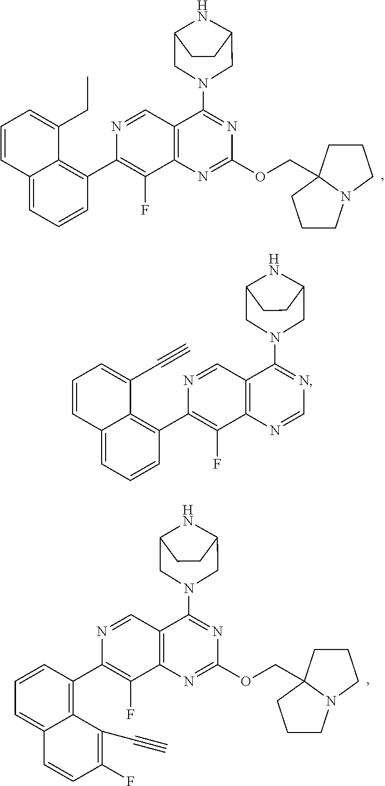 [SMILES: CCc1cccc2cccc(-c3ncc4c(N5CC6CCC(C5)N6)nc(OCC56CCCN5CCC6)nc4c3F)c12 ; C#Cc1cccc2cccc(-c3ncc4c(N5CC6CCC(C5)N6)ncnc4c3F)c12 ; C#Cc1c(F)ccc2cccc(-c3ncc4c(N5CC6CCC(C5)N6)nc(OCC56CCCN5CCC6)nc4c3F)c12]

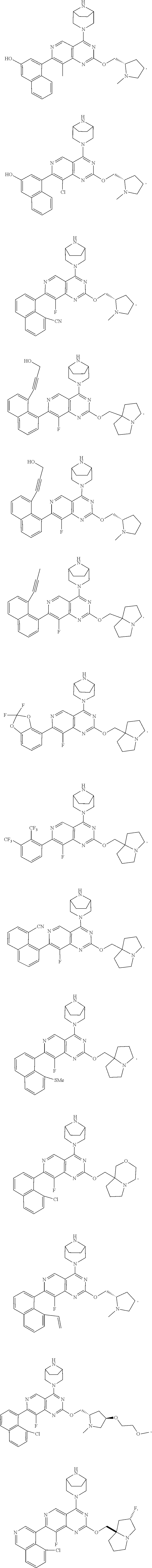

[chemical structure]

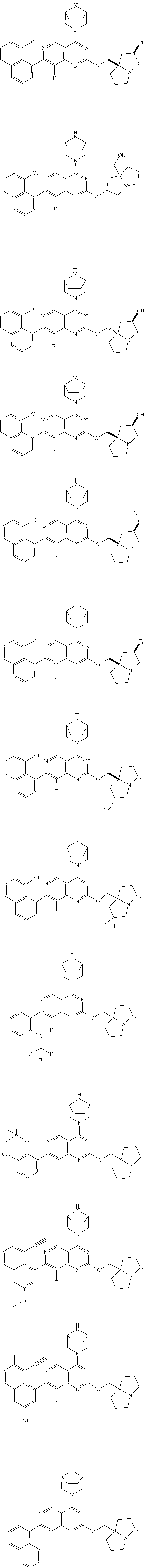

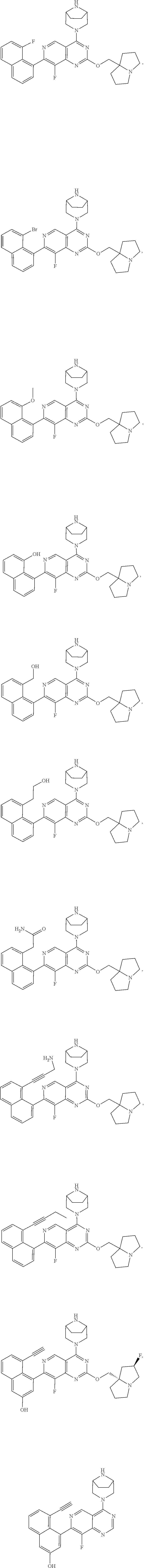

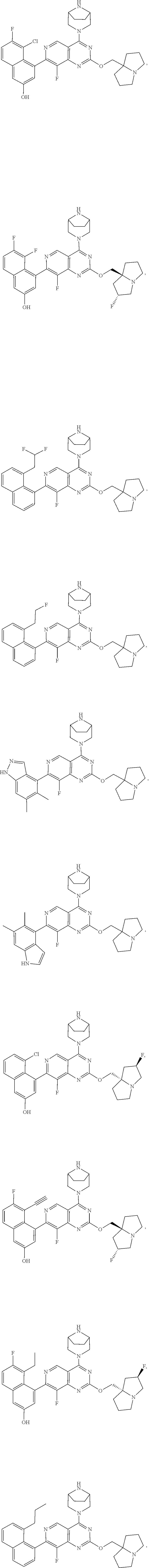

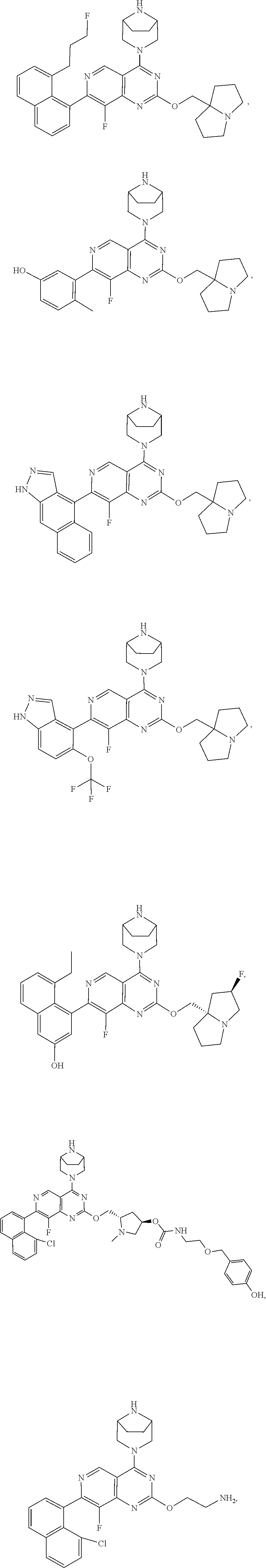

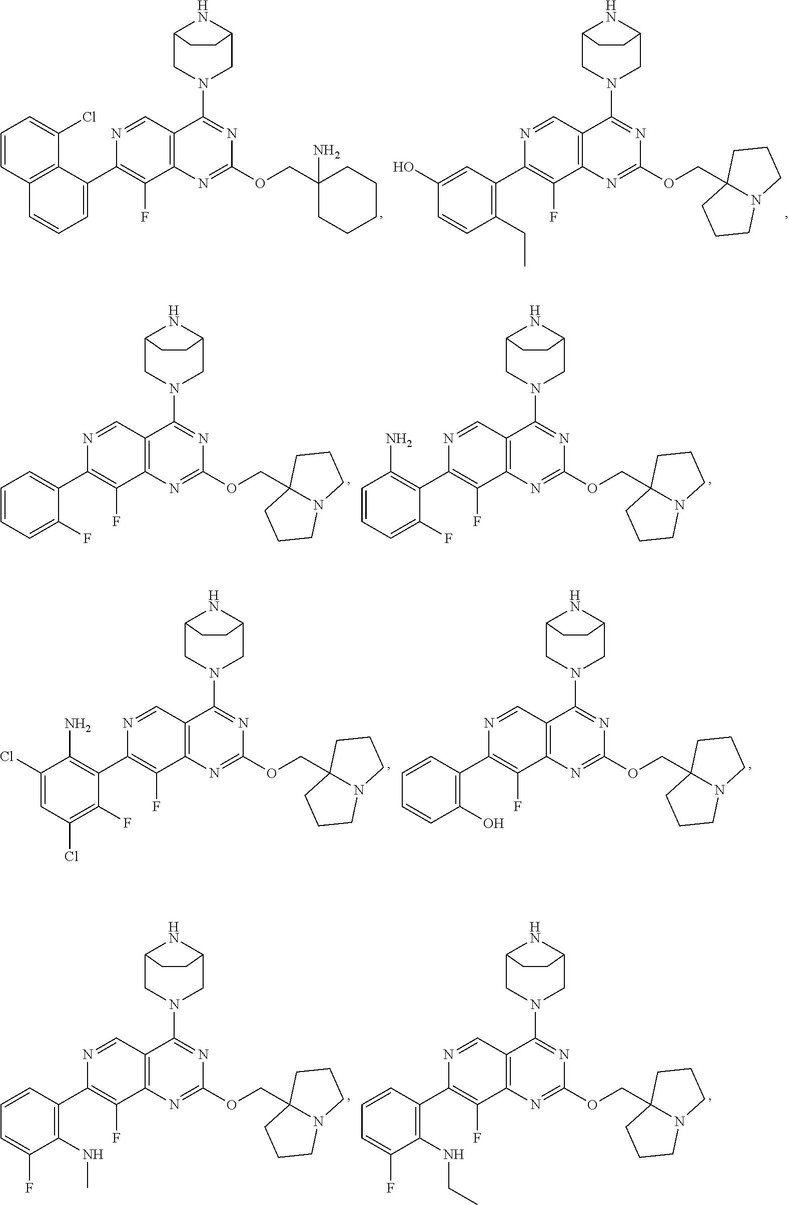

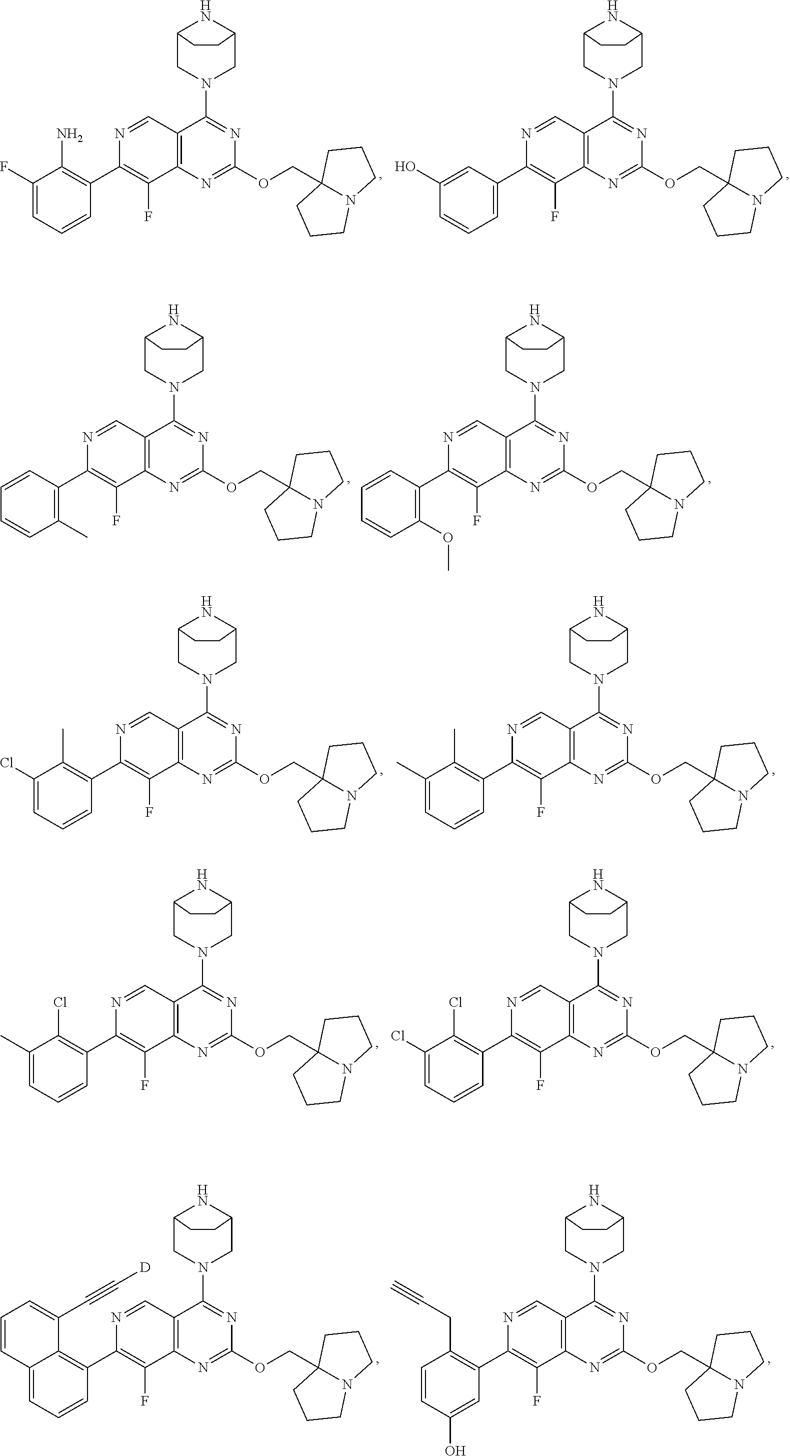

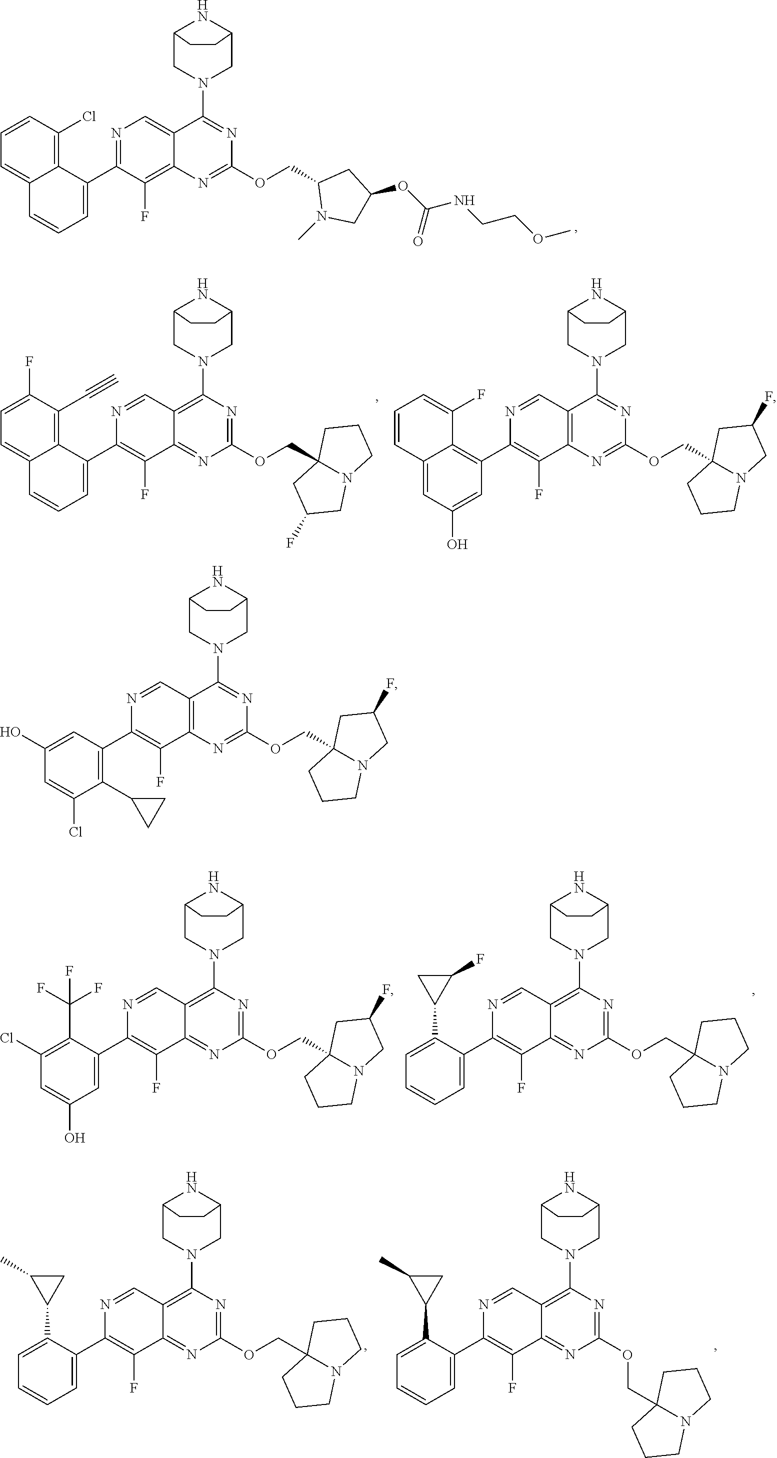

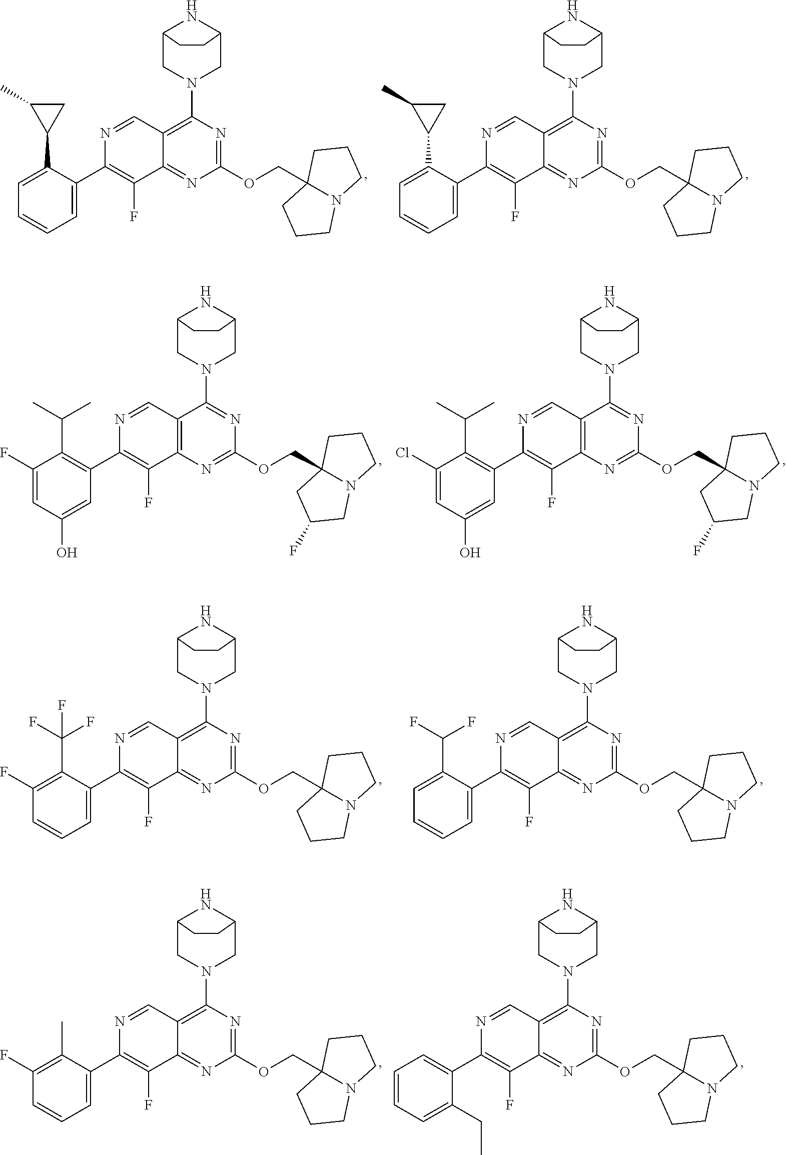

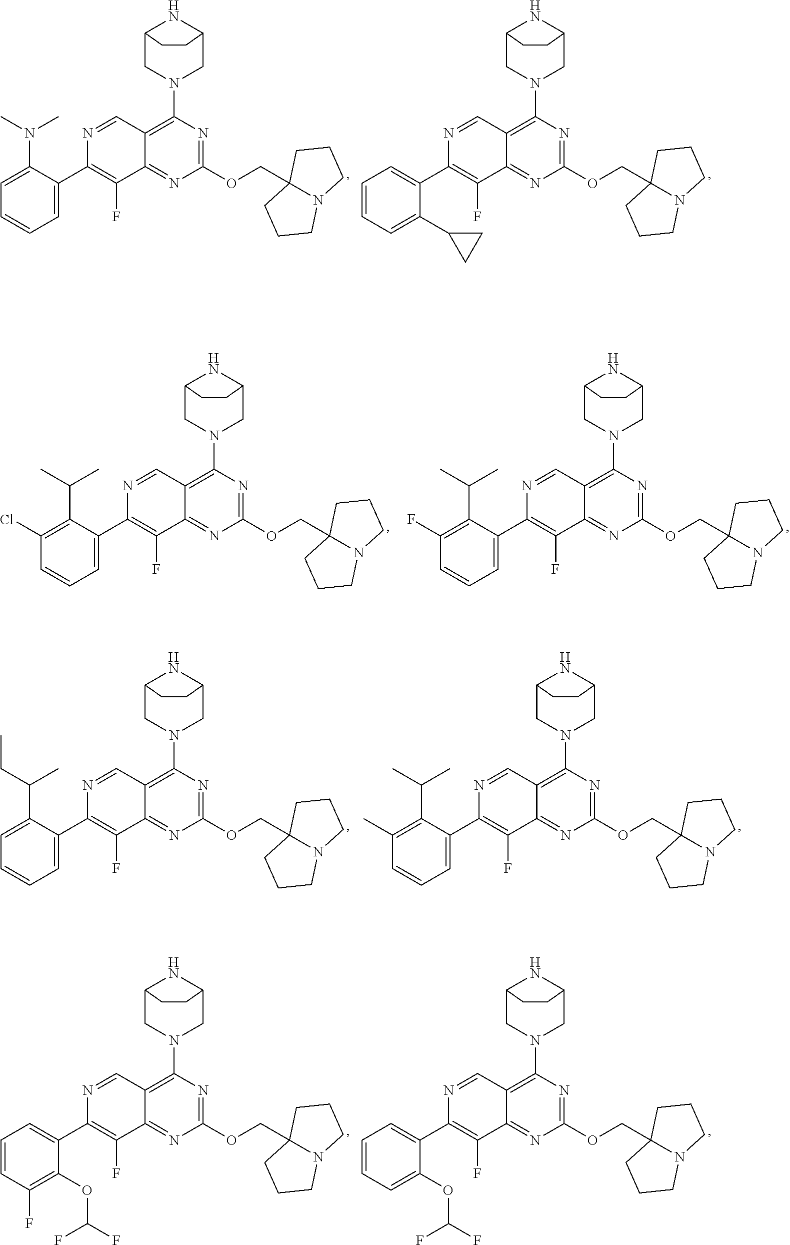

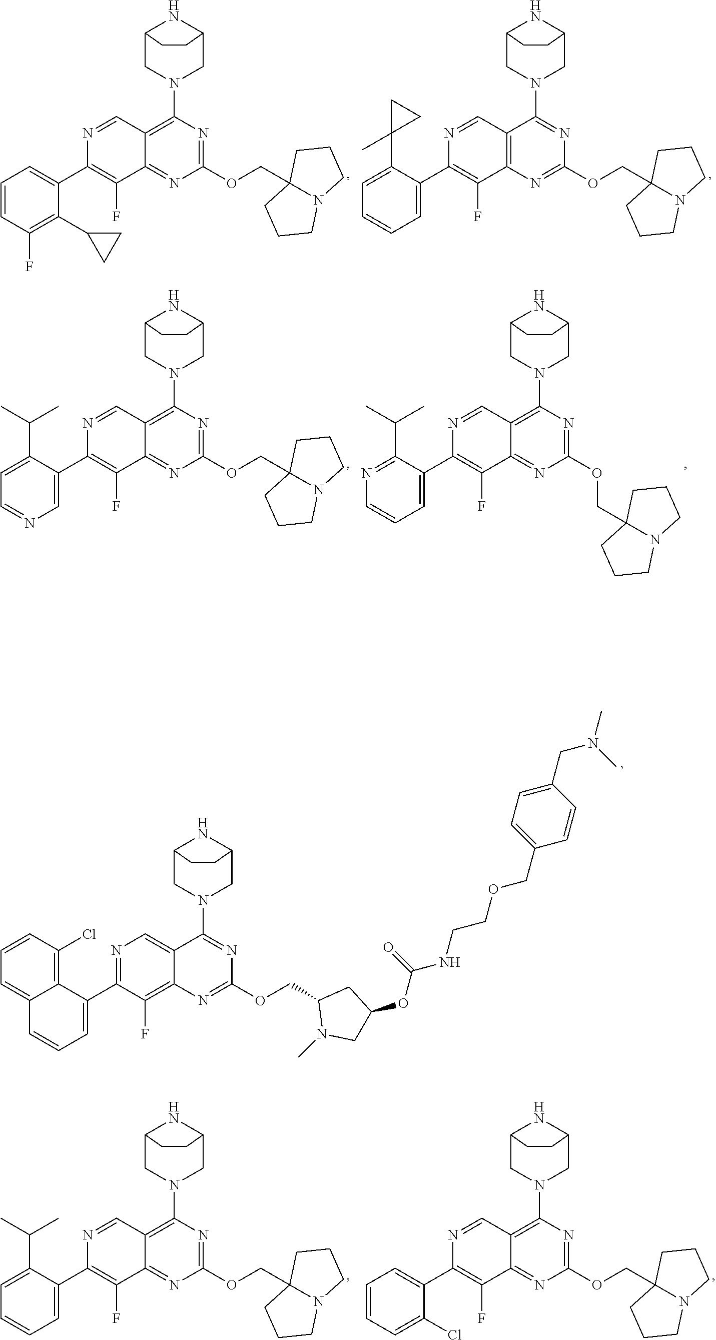

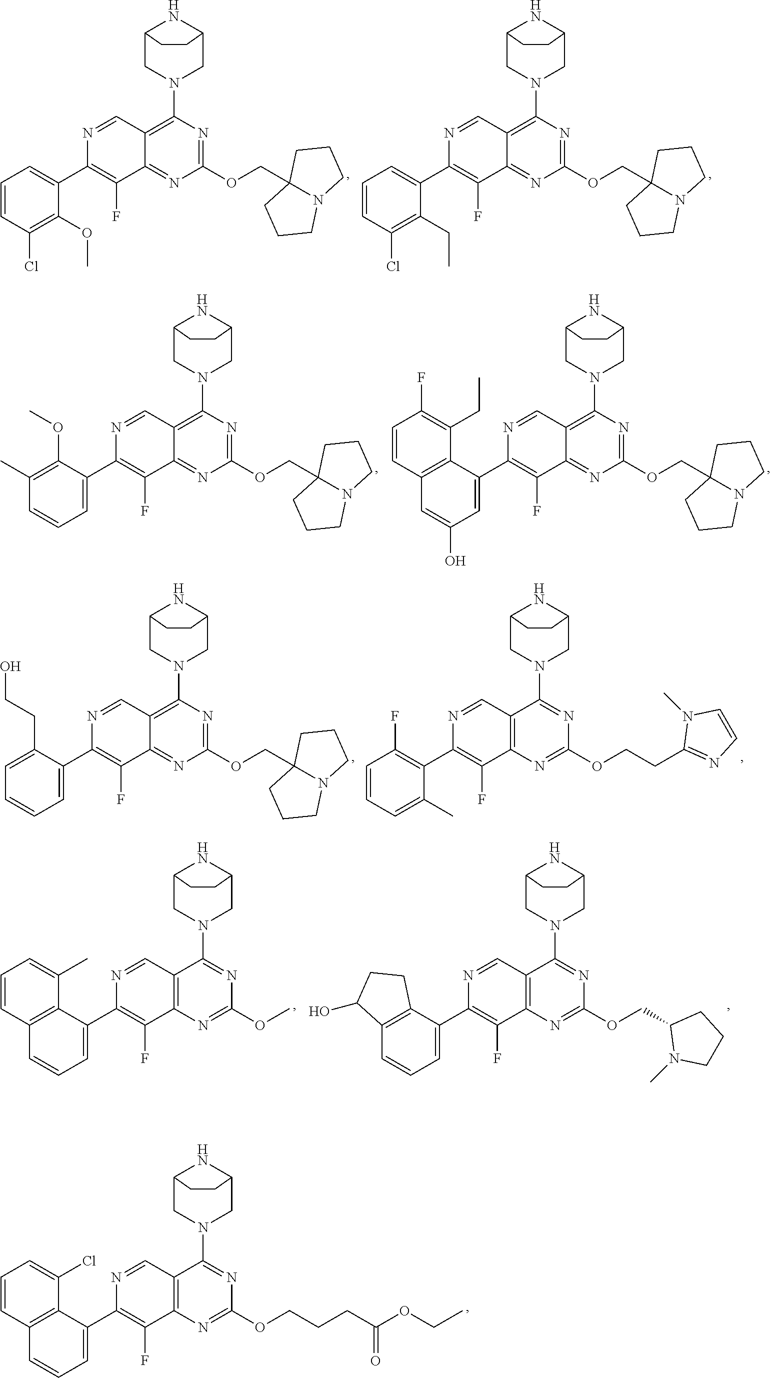

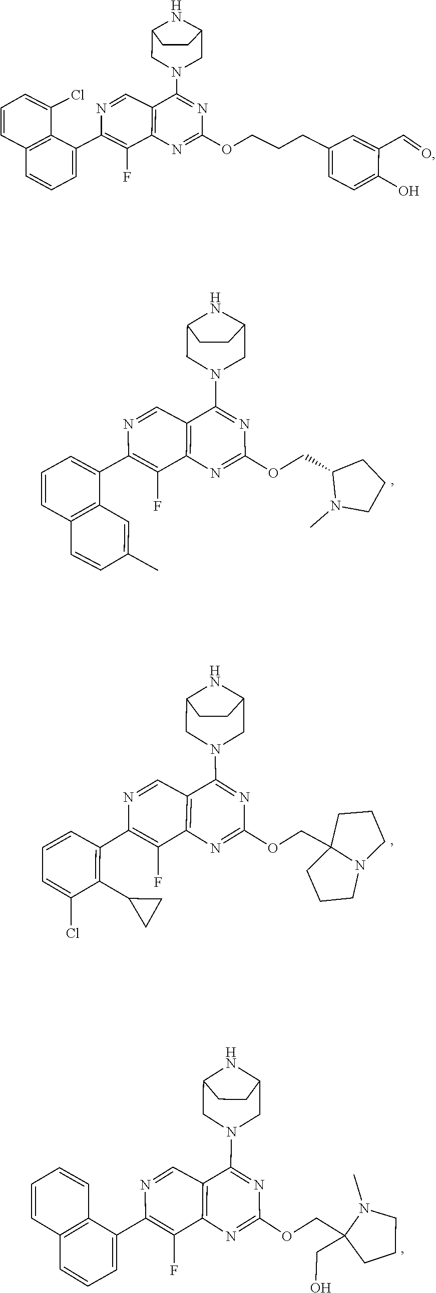 [SMILES: O=Cc1cc(CCCOc2nc(N3CC4CCC(C3)N4)c3cnc(-c4cccc5cccc(Cl)c45)c(F)c3n2)ccc1O ; Cc1ccc2cccc(-c3ncc4c(N5CC6CCC(C5)N6)nc(OC[C@@H]5CCCN5C)nc4c3F)c2c1 ; Fc1c(-c2cccc(Cl)c2C2CC2)ncc2c(N3CC4CCC(C3)N4)nc(OCC34CCCN3CCC4)nc12 ; CN1CCCC1(CO)COc1nc(N2CC3CCC(C2)N3)c2cnc(-c3cccc4ccccc34)c(F)c2n1]

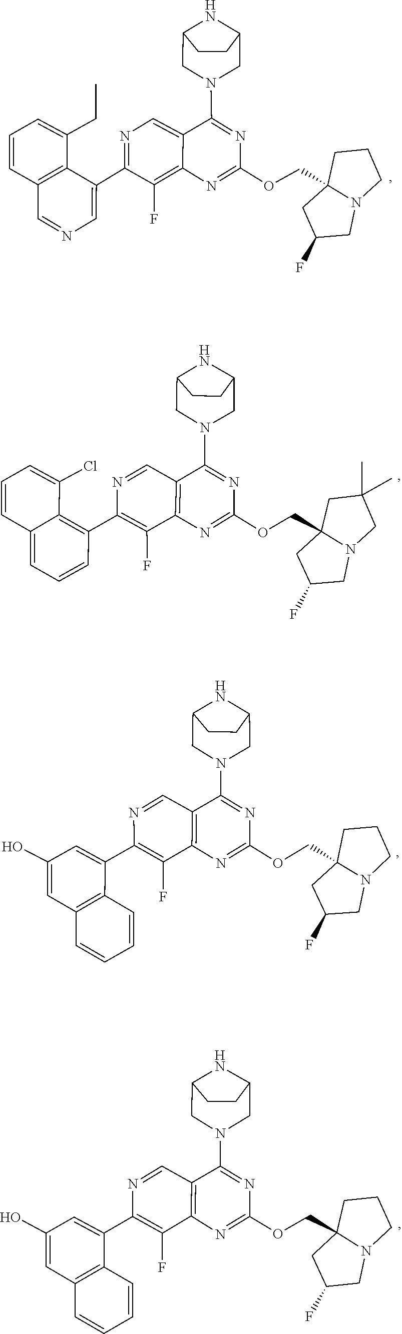 [SMILES: CCc1cccc2cncc(-c3ncc4c(N5CC6CCC(C5)N6)nc(OC[C@]56CCCN5C[C@@H](F)C6)nc4c3F)c12 ; CC1(C)CN2C[C@H](F)C[C@]2(COc2nc(N3CC4CCC(C3)N4)c3cnc(-c4cccc5cccc(Cl)c45)c(F)c3n2)C1 ; Oc1cc(-c2ncc3c(N4CC5CCC(C4)N5)nc(OC[C@]45CCCN4C[C@@H](F)C5)nc3c2F)c2ccccc2c1 ; Oc1cc(-c2ncc3c(N4CC5CCC(C4)N5)nc(OC[C@@]45CCCN4C[C@H](F)C5)nc3c2F)c2ccccc2c1]

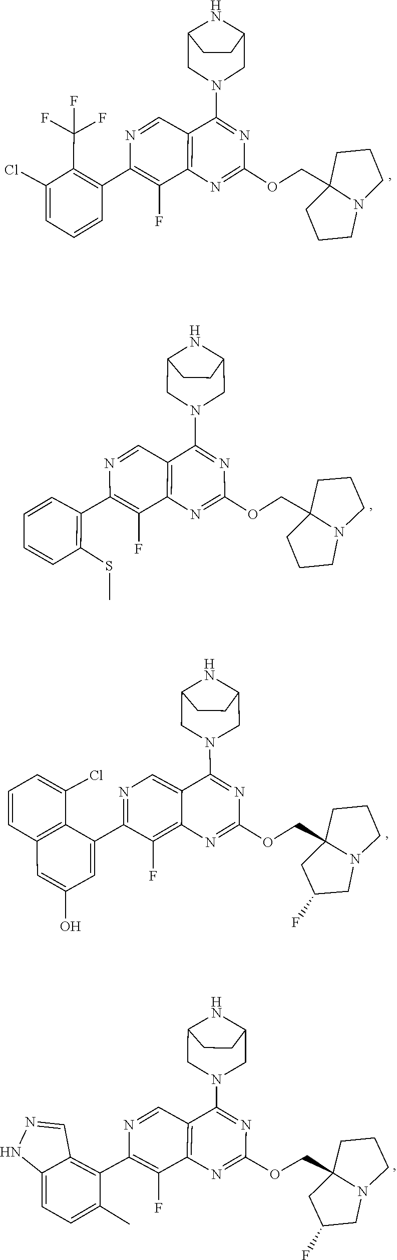 [SMILES: Fc1c(-c2cccc(Cl)c2C(F)(F)F)ncc2c(N3CC4CCC(C3)N4)nc(OCC34CCCN3CCC4)nc12 ; CSc1ccccc1-c1ncc2c(N3CC4CCC(C3)N4)nc(OCC34CCCN3CCC4)nc2c1F ; Oc1cc(-c2ncc3c(N4CC5CCC(C4)N5)nc(OC[C@@]45CCCN4C[C@H](F)C5)nc3c2F)c2c(Cl)cccc2c1 ; Cc1ccc2[nH]ncc2c1-c1ncc2c(N3CC4CCC(C3)N4)nc(OC[C@@]34CCCN3C[C@H](F)C4)nc2c1F]

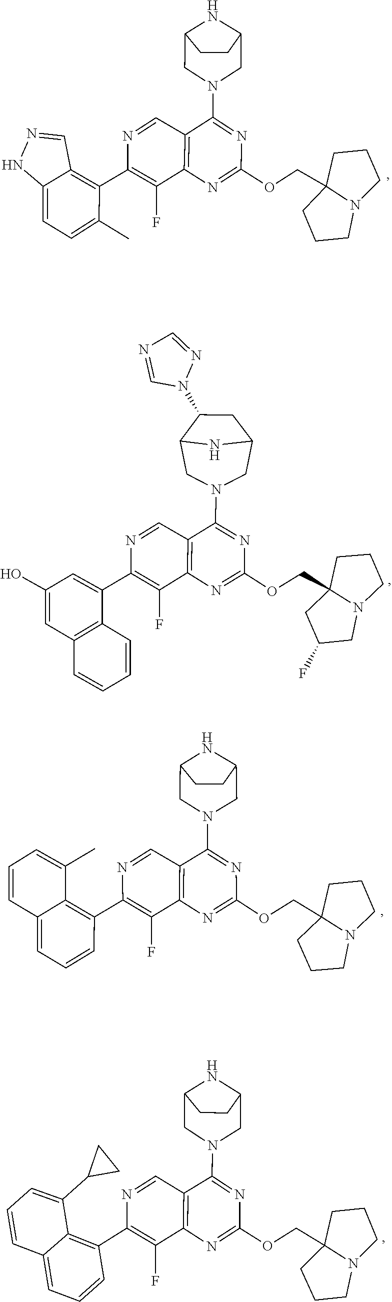 [SMILES: Cc1ccc2[nH]ncc2c1-c1ncc2c(N3CC4CCC(C3)N4)nc(OCC34CCCN3CCC4)nc2c1F ; Oc1cc(-c2ncc3c(N4CC5C[C@@H](n6cncn6)C(C4)N5)nc(OC[C@@]45CCCN4C[C@H](F)C5)nc3c2F)c2ccccc2c1 ; Cc1cccc2cccc(-c3ncc4c(N5CC6CCC(C5)N6)nc(OCC56CCCN5CCC6)nc4c3F)c12 ; Fc1c(-c2cccc3cccc(C4CC4)c23)ncc2c(N3CC4CCC(C3)N4)nc(OCC34CCCN3CCC4)nc12]

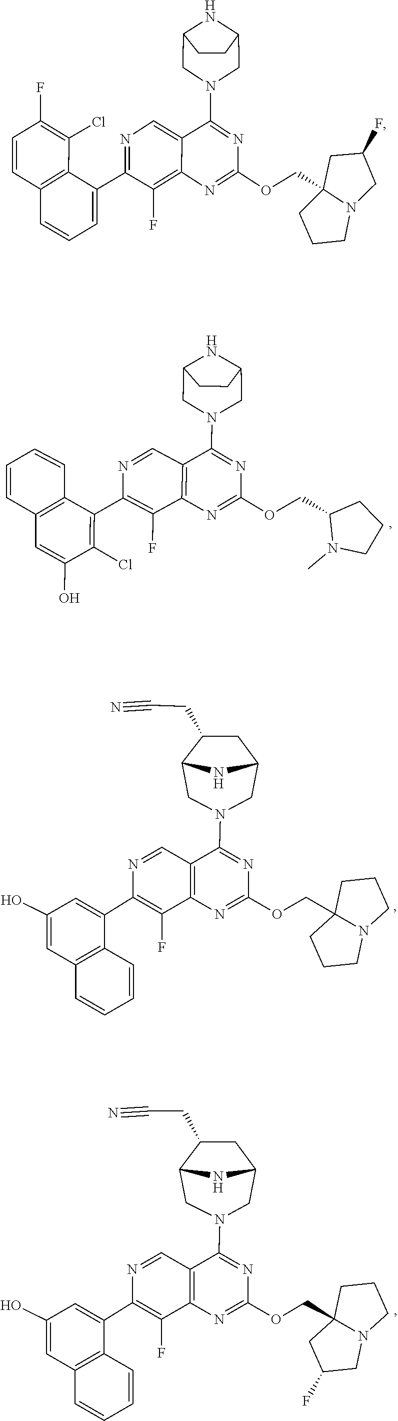 [SMILES: Fc1ccc2cccc(-c3ncc4c(N5CC6CCC(C5)N6)nc(OC[C@@]56CCCN5C[C@H](F)C6)nc4c3F)c2c1Cl ; CN1CCC[C@H]1COc1nc(N2CC3CCC(C2)N3)c2cnc(-c3c(Cl)c(O)cc4ccccc34)c(F)c2n1 ; N#CC[C@@H]1C[C@@H]2CN(c3nc(OCC45CCCN4CCC5)nc4c(F)c(-c5cc(O)cc6ccccc56)ncc34)C[C@H]1N2 ; N#CC[C@@H]1C[C@@H]2CN(c3nc(OC[C@@]45CCCN4C[C@H](F)C5)nc4c(F)c(-c5cc(O)cc6ccccc56)ncc34)C[C@H]1N2]

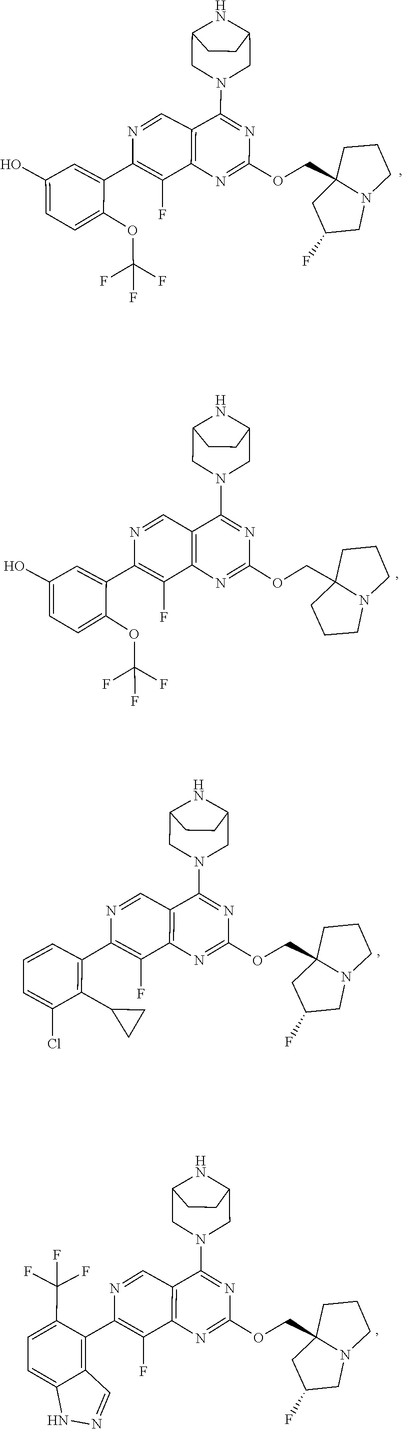 [SMILES: Oc1ccc(OC(F)(F)F)c(-c2ncc3c(N4CC5CCC(C4)N5)nc(OC[C@@]45CCCN4C[C@H](F)C5)nc3c2F)c1 ; Oc1ccc(OC(F)(F)F)c(-c2ncc3c(N4CC5CCC(C4)N5)nc(OCC45CCCN4CCC5)nc3c2F)c1 ; Fc1c(-c2cccc(Cl)c2C2CC2)ncc2c(N3CC4CCC(C3)N4)nc(OC[C@@]34CCCN3C[C@H](F)C4)nc12 ; Fc1c(-c2c(C(F)(F)F)ccc3[nH]ncc23)ncc2c(N3CC4CCC(C3)N4)nc(OC[C@@]34CCCN3C[C@H](F)C4)nc12]

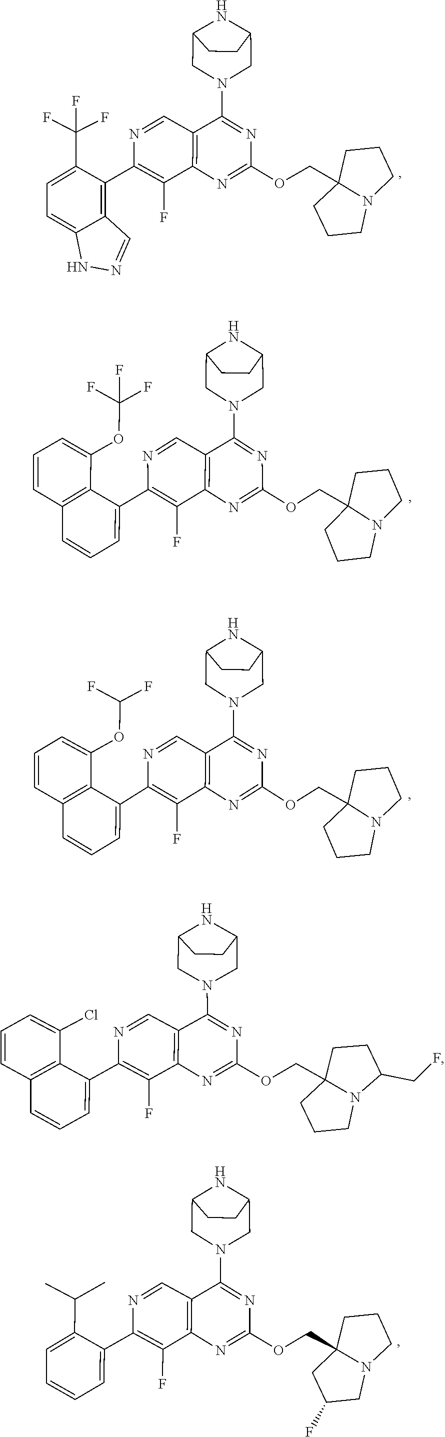 [SMILES: Fc1c(-c2c(C(F)(F)F)ccc3[nH]ncc23)ncc2c(N3CC4CCC(C3)N4)nc(OCC34CCCN3CCC4)nc12 ; Fc1c(-c2cccc3cccc(OC(F)(F)F)c23)ncc2c(N3CC4CCC(C3)N4)nc(OCC34CCCN3CCC4)nc12 ; Fc1c(-c2cccc3cccc(OC(F)F)c23)ncc2c(N3CC4CCC(C3)N4)nc(OCC34CCCN3CCC4)nc12 ; FCC1CCC2(COc3nc(N4CC5CCC(C4)N5)c4cnc(-c5cccc6cccc(Cl)c56)c(F)c4n3)CCCN12 ; CC(C)c1ccccc1-c1ncc2c(N3CC4CCC(C3)N4)nc(OC[C@@]34CCCN3C[C@H](F)C4)nc2c1F]

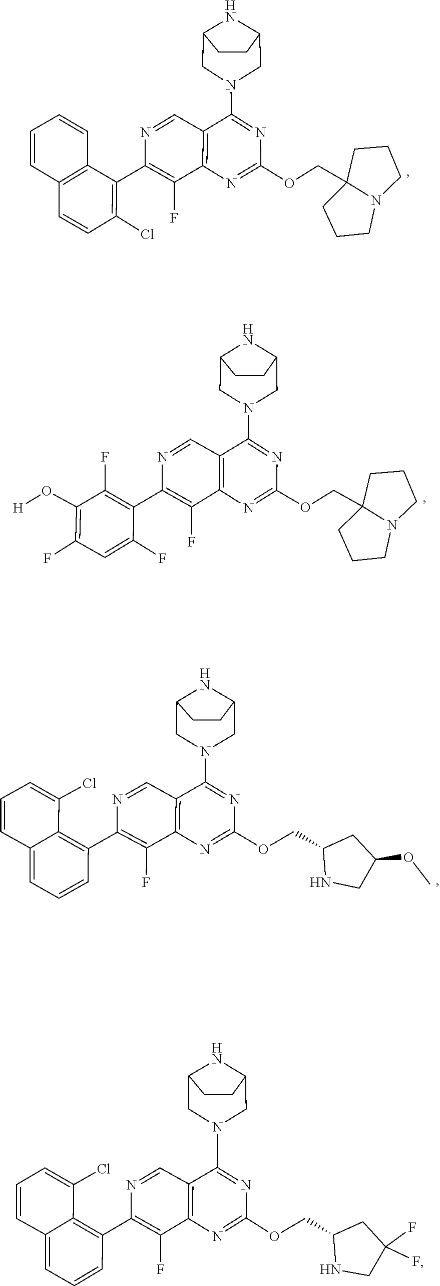 [SMILES: Fc1c(-c2c(Cl)ccc3ccccc23)ncc2c(N3CC4CCC(C3)N4)nc(OCC34CCCN3CCC4)nc12 ; Oc1c(F)cc(F)c(-c2ncc3c(N4CC5CCC(C4)N5)nc(OCC45CCCN4CCC5)nc3c2F)c1F ; CO[C@H]1CN[C@H](COc2nc(N3CC4CCC(C3)N4)c3cnc(-c4cccc5cccc(Cl)c45)c(F)c3n2)C1 ; Fc1c(-c2cccc3cccc(Cl)c23)ncc2c(N3CC4CCC(C3)N4)nc(OC[C@@H]3CC(F)(F)CN3)nc12]

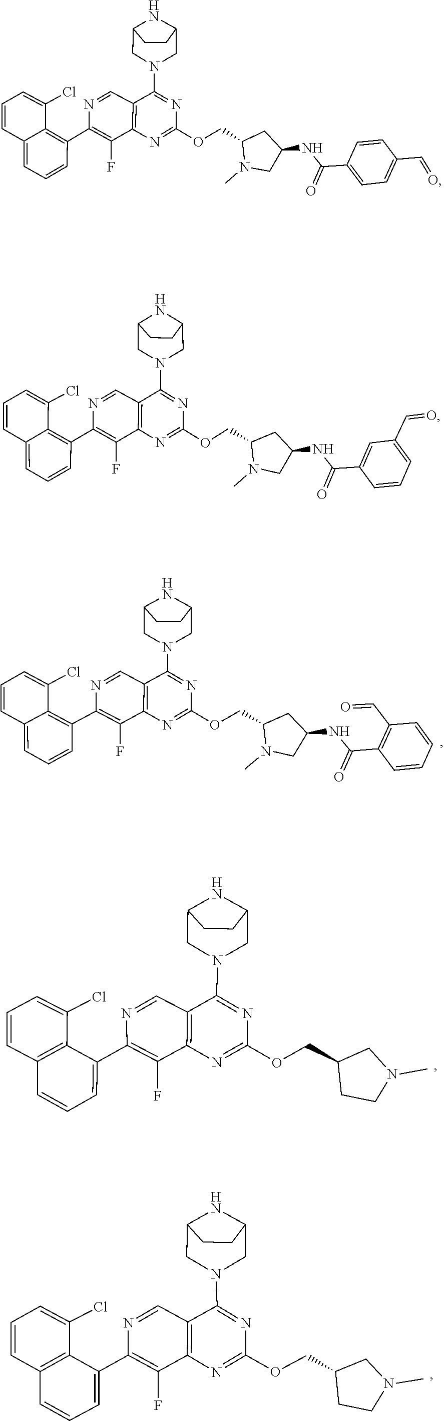 [SMILES: CN1C[C@H](NC(=O)c2ccc(C=O)cc2)C[C@H]1COc1nc(N2CC3CCC(C2)N3)c2cnc(-c3cccc4cccc(Cl)c34)c(F)c2n1 ; CN1C[C@H](NC(=O)c2cccc(C=O)c2)C[C@H]1COc1nc(N2CC3CCC(C2)N3)c2cnc(-c3cccc4cccc(Cl)c34)c(F)c2n1 ; CN1C[C@H](NC(=O)c2ccccc2C=O)C[C@H]1COc1nc(N2CC3CCC(C2)N3)c2cnc(-c3cccc4cccc(Cl)c34)c(F)c2n1 ; CN1CC[C@@H](COc2nc(N3CC4CCC(C3)N4)c3cnc(-c4cccc5cccc(Cl)c45)c(F)c3n2)C1 ; CN1CC[C@H](COc2nc(N3CC4CCC(C3)N4)c3cnc(-c4cccc5cccc(Cl)c45)c(F)c3n2)C1]

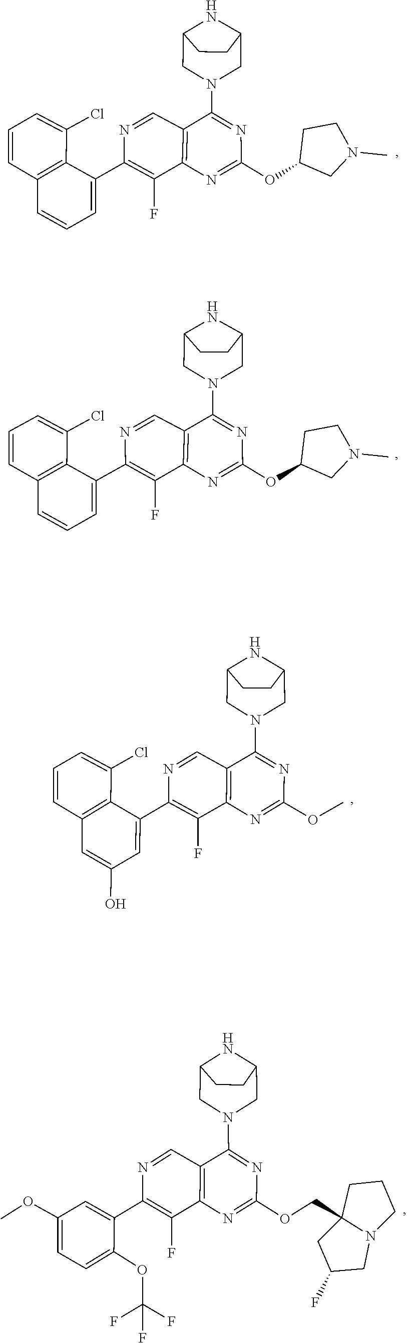

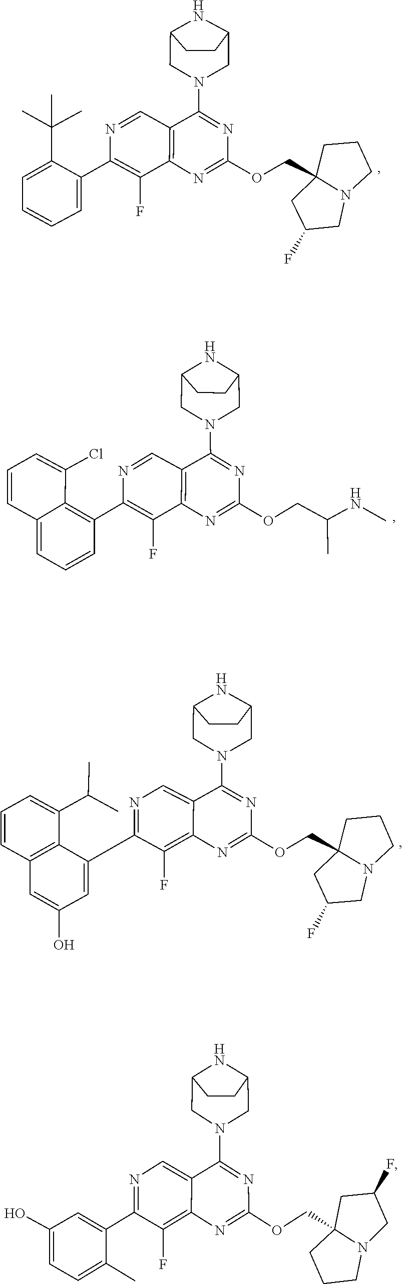 [SMILES: CC(C)(C)c1ccccc1-c1ncc2c(N3CC4CCC(C3)N4)nc(OC[C@@]34CCCN3C[C@H](F)C4)nc2c1F ; CNC(C)COc1nc(N2CC3CCC(C2)N3)c2cnc(-c3cccc4cccc(Cl)c34)c(F)c2n1 ; CC(C)c1cccc2cc(O)cc(-c3ncc4c(N5CC6CCC(C5)N6)nc(OC[C@@]56CCCN5C[C@H](F)C6)nc4c3F)c12 ; Cc1ccc(O)cc1-c1ncc2c(N3CC4CCC(C3)N4)nc(OC[C@@]34CCCN3C[C@H](F)C4)nc2c1F]

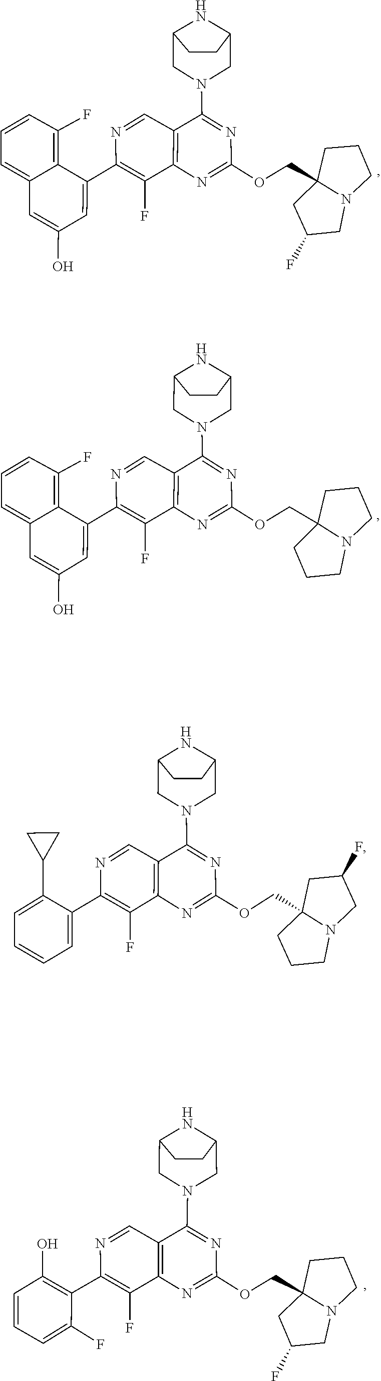 [SMILES: Oc1cc(-c2ncc3c(N4CC5CCC(C4)N5)nc(OC[C@@]45CCCN4C[C@H](F)C5)nc3c2F)c2c(F)cccc2c1 ; Oc1cc(-c2ncc3c(N4CC5CCC(C4)N5)nc(OCC45CCCN4CCC5)nc3c2F)c2c(F)cccc2c1 ; Fc1c(-c2ccccc2C2CC2)ncc2c(N3CC4CCC(C3)N4)nc(OC[C@@]34CCCN3C[C@H](F)C4)nc12 ; Oc1cccc(F)c1-c1ncc2c(N3CC4CCC(C3)N4)nc(OC[C@@]34CCCN3C[C@H](F)C4)nc2c1F]

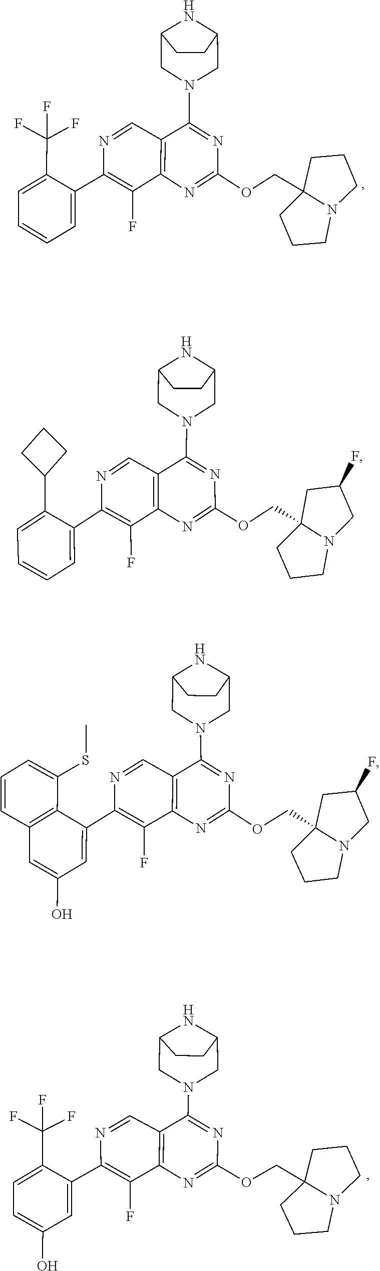 [SMILES: Fc1c(-c2ccccc2C(F)(F)F)ncc2c(N3CC4CCC(C3)N4)nc(OCC34CCCN3CCC4)nc12 ; Fc1c(-c2ccccc2C2CCC2)ncc2c(N3CC4CCC(C3)N4)nc(OC[C@@]34CCCN3C[C@H](F)C4)nc12 ; CSc1cccc2cc(O)cc(-c3ncc4c(N5CC6CCC(C5)N6)nc(OC[C@@]56CCCN5C[C@H](F)C6)nc4c3F)c12 ; Oc1ccc(C(F)(F)F)c(-c2ncc3c(N4CC5CCC(C4)N5)nc(OCC45CCCN4CCC5)nc3c2F)c1]

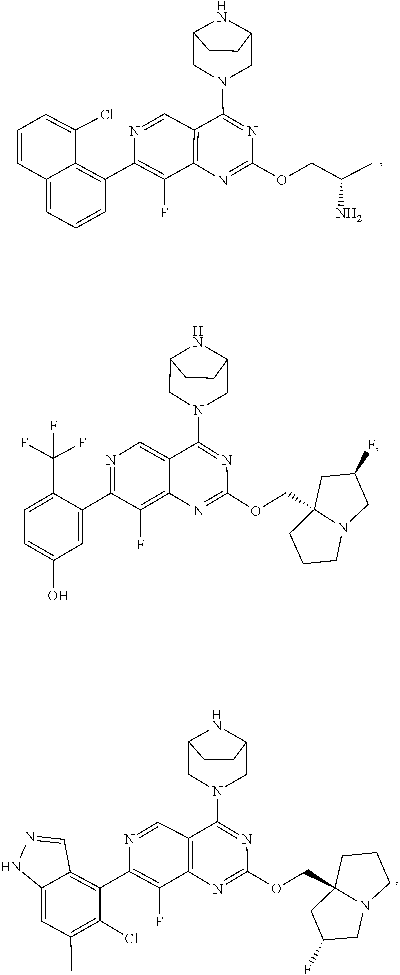 [SMILES: C[C@H](N)COc1nc(N2CC3CCC(C2)N3)c2cnc(-c3cccc4cccc(Cl)c34)c(F)c2n1 ; Oc1ccc(C(F)(F)F)c(-c2ncc3c(N4CC5CCC(C4)N5)nc(OC[C@@]45CCCN4C[C@H](F)C5)nc3c2F)c1 ; Cc1cc2[nH]ncc2c(-c2ncc3c(N4CC5CCC(C4)N5)nc(OC[C@@]45CCCN4C[C@H](F)C5)nc3c2F)c1Cl]

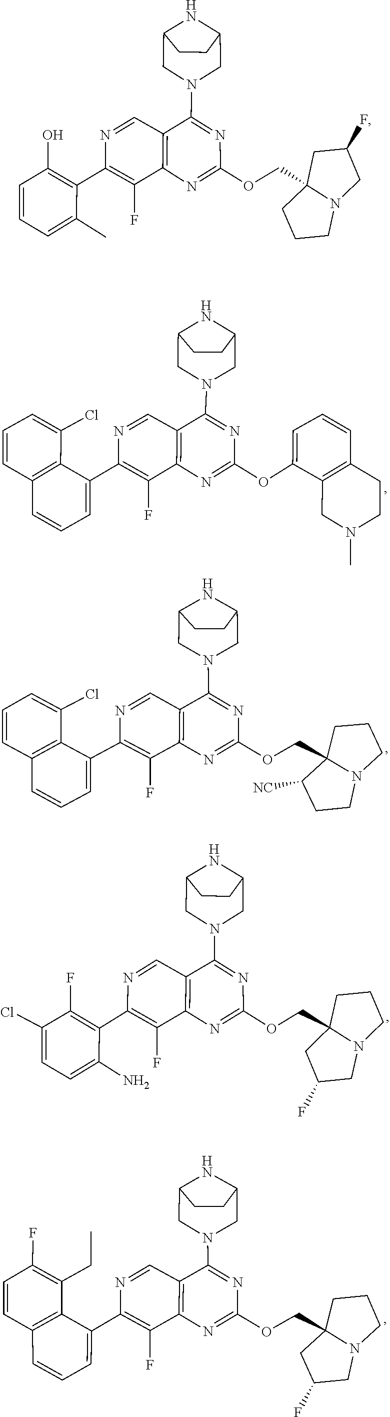 [SMILES: Cc1cccc(O)c1-c1ncc2c(N3CC4CCC(C3)N4)nc(OC[C@@]34CCCN3C[C@H](F)C4)nc2c1F ; CN1CCc2cccc(Oc3nc(N4CC5CCC(C4)N5)c4cnc(-c5cccc6cccc(Cl)c56)c(F)c4n3)c2C1 ; N#C[C@H]1CCN2CCC[C@@]12COc1nc(N2CC3CCC(C2)N3)c2cnc(-c3cccc4cccc(Cl)c34)c(F)c2n1 ; Nc1ccc(Cl)c(F)c1-c1ncc2c(N3CC4CCC(C3)N4)nc(OC[C@@]34CCCN3C[C@H](F)C4)nc2c1F ; CCc1c(F)ccc2cccc(-c3ncc4c(N5CC6CCC(C5)N6)nc(OC[C@@]56CCCN5C[C@H](F)C6)nc4c3F)c12]

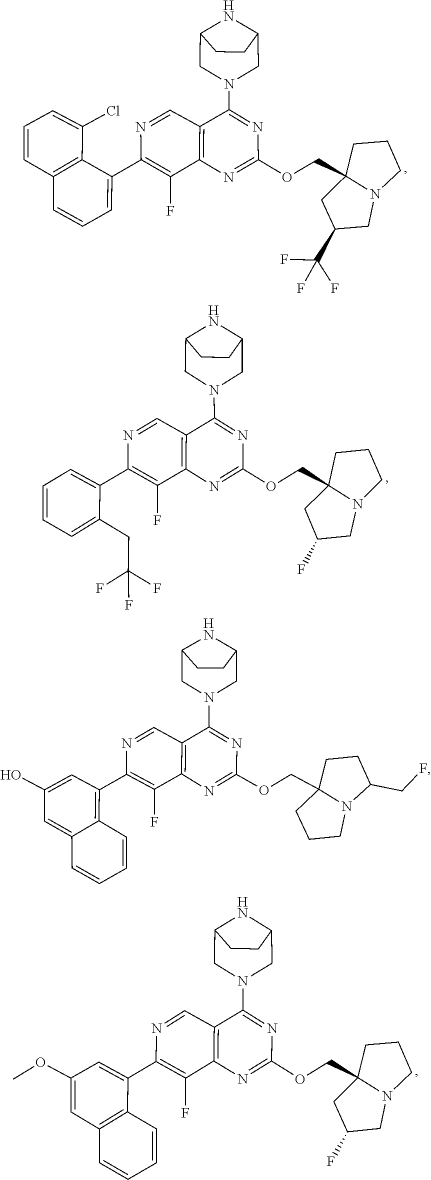 [SMILES: Fc1c(-c2cccc3cccc(Cl)c23)ncc2c(N3CC4CCC(C3)N4)nc(OC[C@@]34CCCN3C[C@@H](C(F)(F)F)C4)nc12 ; Fc1c(-c2ccccc2CC(F)(F)F)ncc2c(N3CC4CCC(C3)N4)nc(OC[C@@]34CCCN3C[C@H](F)C4)nc12 ; Oc1cc(-c2ncc3c(N4CC5CCC(C4)N5)nc(OCC45CCCN4C(CF)CC5)nc3c2F)c2ccccc2c1 ; COc1cc(-c2ncc3c(N4CC5CCC(C4)N5)nc(OC[C@@]45CCCN4C[C@H](F)C5)nc3c2F)c2ccccc2c1]

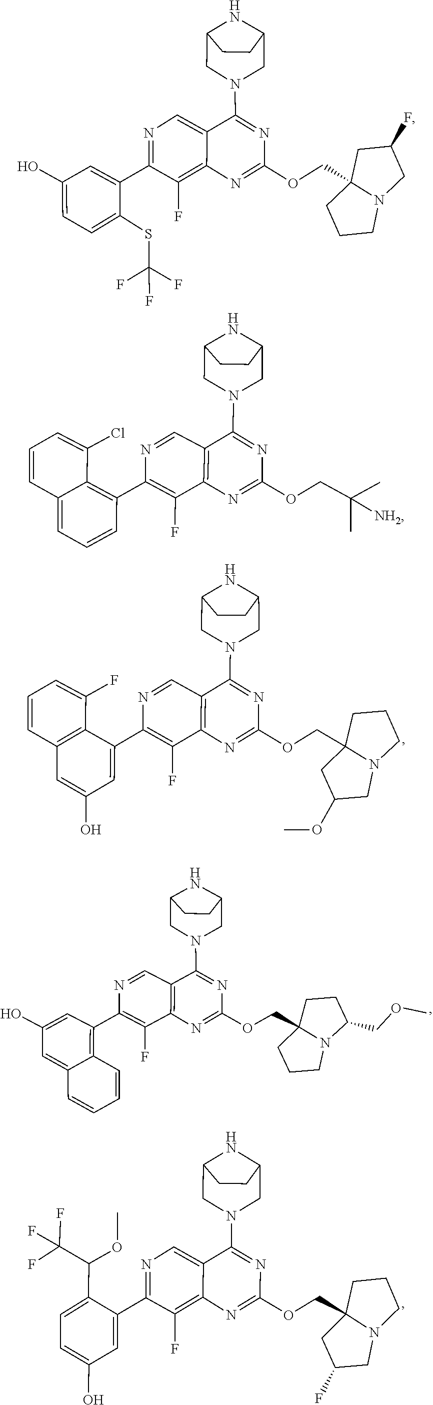 [SMILES: Oc1ccc(SC(F)(F)F)c(-c2ncc3c(N4CC5CCC(C4)N5)nc(OC[C@@]45CCCN4C[C@H](F)C5)nc3c2F)c1 ; CC(C)(N)COc1nc(N2CC3CCC(C2)N3)c2cnc(-c3cccc4cccc(Cl)c34)c(F)c2n1 ; COC1CN2CCCC2(COc2nc(N3CC4CCC(C3)N4)c3cnc(-c4cc(O)cc5cccc(F)c45)c(F)c3n2)C1 ; COC[C@H]1CC[C@@]2(COc3nc(N4CC5CCC(C4)N5)c4cnc(-c5cc(O)cc6ccccc56)c(F)c4n3)CCCN12 ; COC(c1ccc(O)cc1-c1ncc2c(N3CC4CCC(C3)N4)nc(OC[C@@]34CCCN3C[C@H](F)C4)nc2c1F)C(F)(F)F]

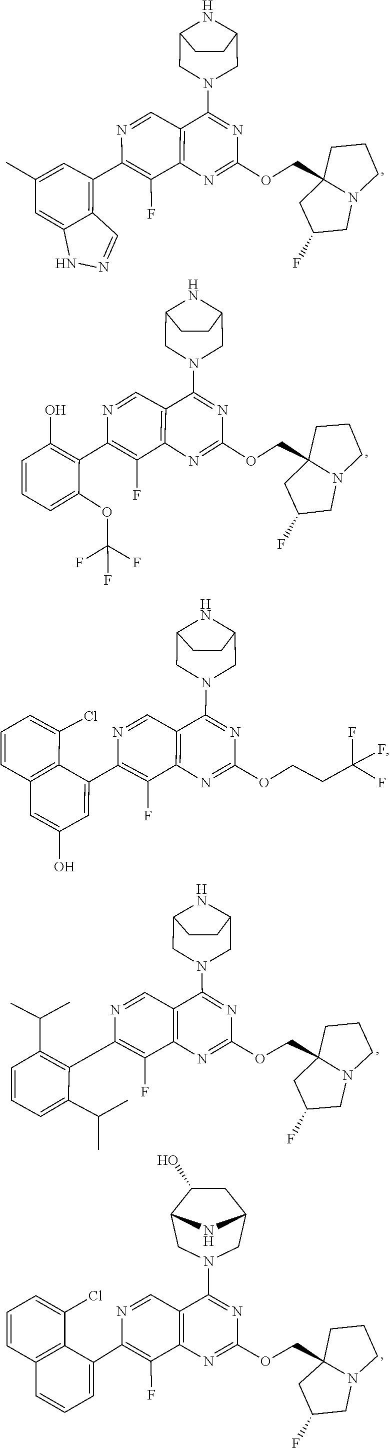 [SMILES: Cc1cc(-c2ncc3c(N4CC5CCC(C4)N5)nc(OC[C@@]45CCCN4C[C@H](F)C5)nc3c2F)c2cn[nH]c2c1 ; Oc1cccc(OC(F)(F)F)c1-c1ncc2c(N3CC4CCC(C3)N4)nc(OC[C@@]34CCCN3C[C@H](F)C4)nc2c1F ; Oc1cc(-c2ncc3c(N4CC5CCC(C4)N5)nc(OCCC(F)(F)F)nc3c2F)c2c(Cl)cccc2c1 ; CC(C)c1cccc(C(C)C)c1-c1ncc2c(N3CC4CCC(C3)N4)nc(OC[C@@]34CCCN3C[C@H](F)C4)nc2c1F ; O[C@@H]1C[C@@H]2CN(c3nc(OC[C@@]45CCCN4C[C@H](F)C5)nc4c(F)c(-c5cccc6cccc(Cl)c56)ncc34)C[C@H]1N2]

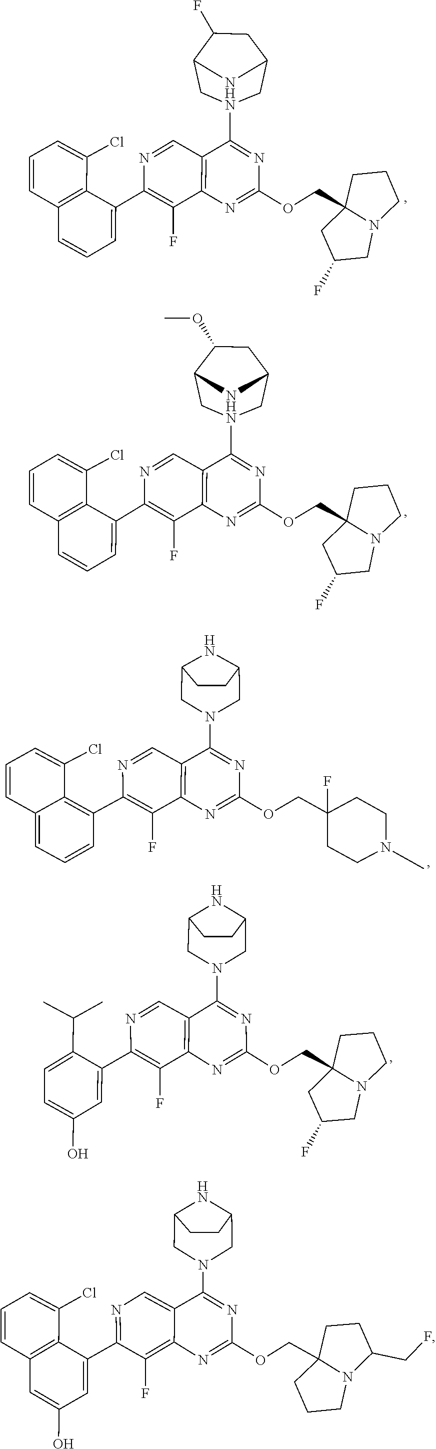

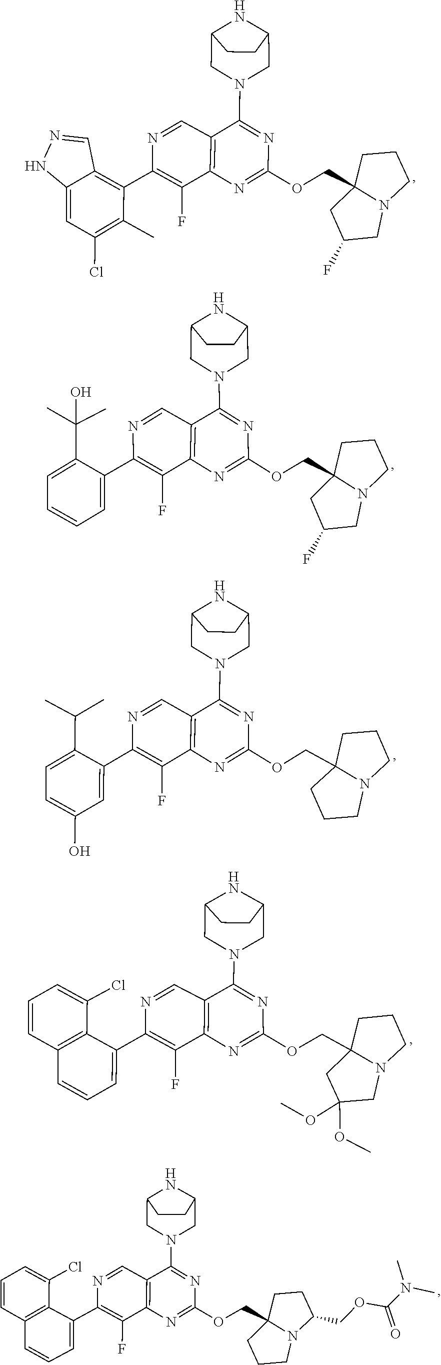 [SMILES: Cc1c(Cl)cc2[nH]ncc2c1-c1ncc2c(N3CC4CCC(C3)N4)nc(OC[C@@]34CCCN3C[C@H](F)C4)nc2c1F ; CC(C)(O)c1ccccc1-c1ncc2c(N3CC4CCC(C3)N4)nc(OC[C@@]34CCCN3C[C@H](F)C4)nc2c1F ; CC(C)c1ccc(O)cc1-c1ncc2c(N3CC4CCC(C3)N4)nc(OCC34CCCN3CCC4)nc2c1F ; COC1(OC)CN2CCCC2(COc2nc(N3CC4CCC(C3)N4)c3cnc(-c4cccc5cccc(Cl)c45)c(F)c3n2)C1 ; CN(C)C(=O)OC[C@H]1CC[C@@]2(COc3nc(N4CC5CCC(C4)N5)c4cnc(-c5cccc6cccc(Cl)c56)c(F)c4n3)CCCN12]

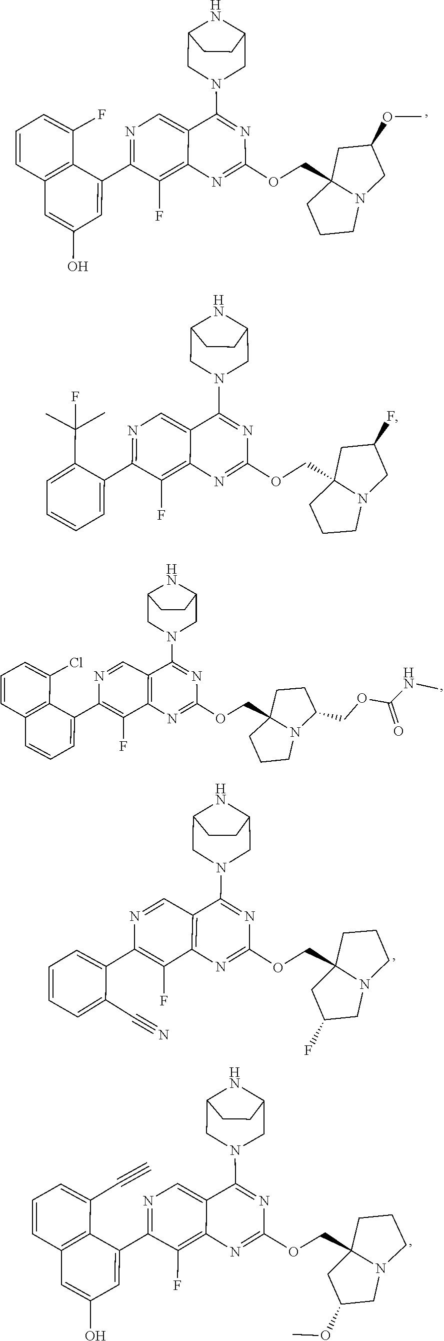

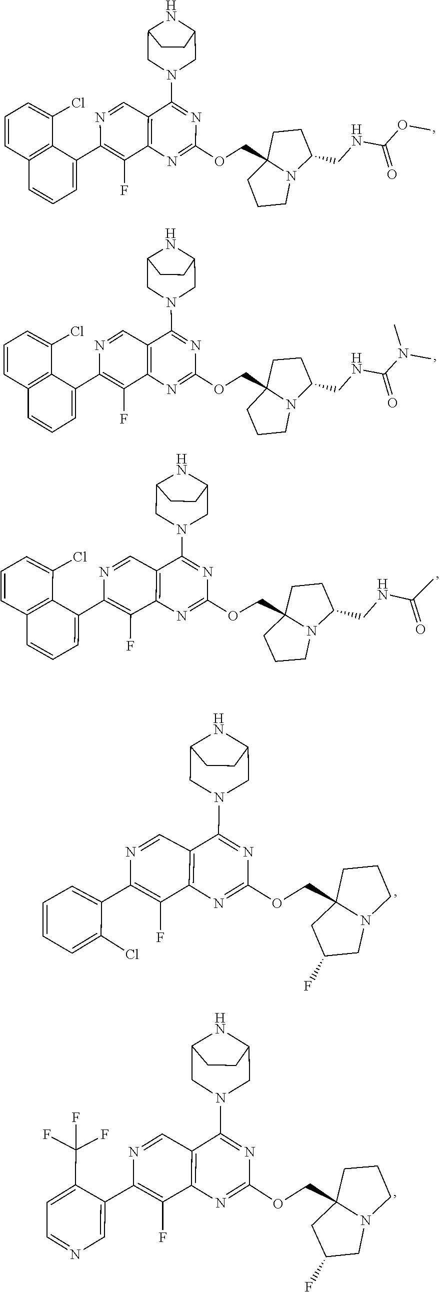 [SMILES: COC(=O)NC[C@H]1CC[C@@]2(COc3nc(N4CC5CCC(C4)N5)c4cnc(-c5cccc6cccc(Cl)c56)c(F)c4n3)CCCN12 ; CN(C)C(=O)NC[C@H]1CC[C@@]2(COc3nc(N4CC5CCC(C4)N5)c4cnc(-c5cccc6cccc(Cl)c56)c(F)c4n3)CCCN12 ; CC(=O)NC[C@H]1CC[C@@]2(COc3nc(N4CC5CCC(C4)N5)c4cnc(-c5cccc6cccc(Cl)c56)c(F)c4n3)CCCN12 ; Fc1c(-c2ccccc2Cl)ncc2c(N3CC4CCC(C3)N4)nc(OC[C@@]34CCCN3C[C@H](F)C4)nc12 ; Fc1c(-c2cnccc2C(F)(F)F)ncc2c(N3CC4CCC(C3)N4)nc(OC[C@@]34CCCN3C[C@H](F)C4)nc12]

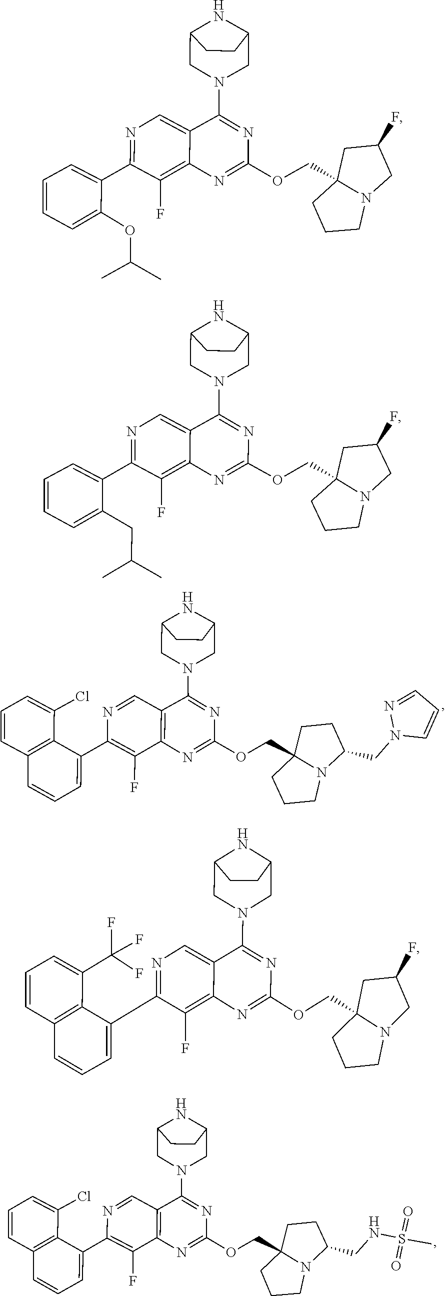

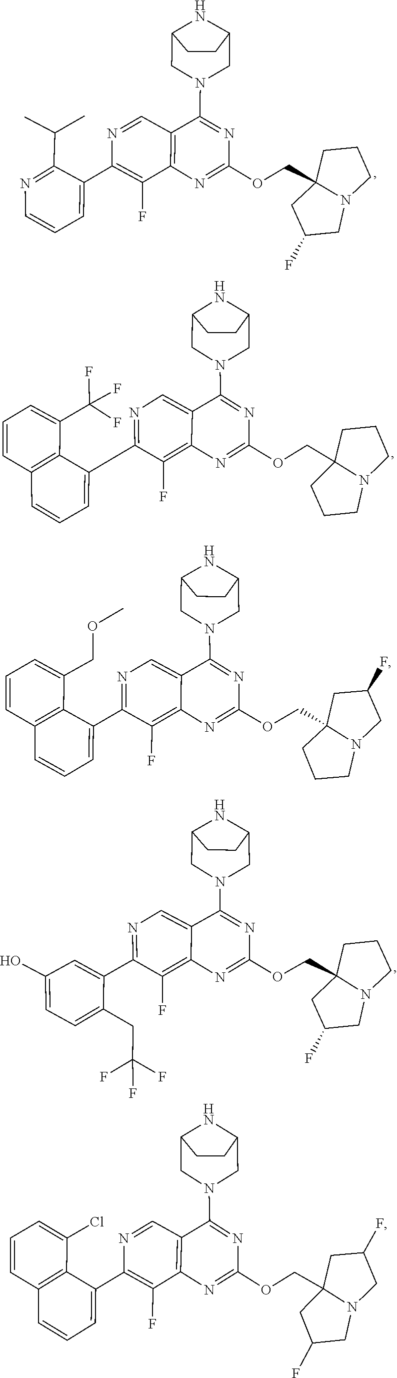 [SMILES: CC(C)c1ncccc1-c1ncc2c(N3CC4CCC(C3)N4)nc(OC[C@@]34CCCN3C[C@H](F)C4)nc2c1F ; Fc1c(-c2cccc3cccc(C(F)(F)F)c23)ncc2c(N3CC4CCC(C3)N4)nc(OCC34CCCN3CCC4)nc12 ; COCc1cccc2cccc(-c3ncc4c(N5CC6CCC(C5)N6)nc(OC[C@@]56CCCN5C[C@H](F)C6)nc4c3F)c12 ; Oc1ccc(CC(F)(F)F)c(-c2ncc3c(N4CC5CCC(C4)N5)nc(OC[C@@]45CCCN4C[C@H](F)C5)nc3c2F)c1 ; Fc1c(-c2cccc3cccc(Cl)c23)ncc2c(N3CC4CCC(C3)N4)nc(OCC34CC(F)CN3CC(F)C4)nc12]

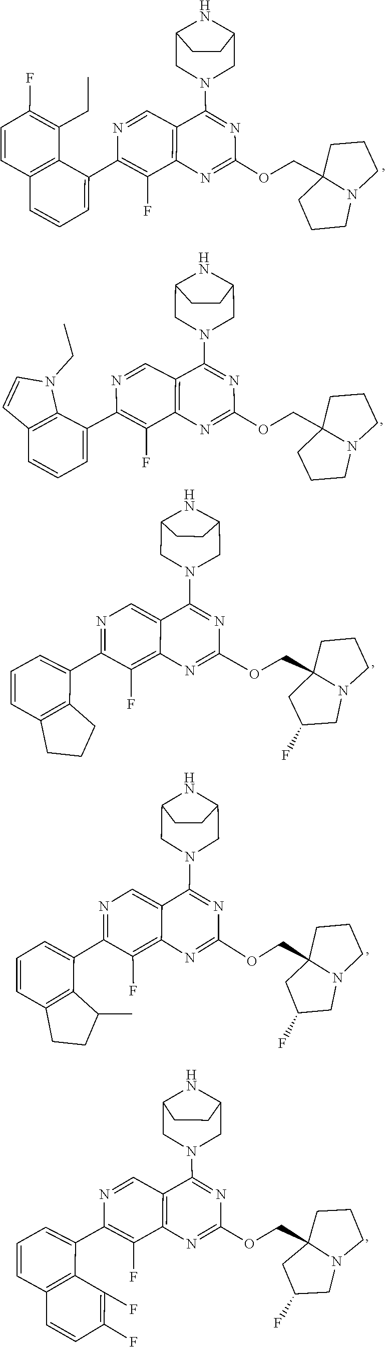 [SMILES: CCc1c(F)ccc2cccc(-c3ncc4c(N5CC6CCC(C5)N6)nc(OCC56CCCN5CCC6)nc4c3F)c12 ; CCn1ccc2cccc(-c3ncc4c(N5CC6CCC(C5)N6)nc(OCC56CCCN5CCC6)nc4c3F)c21 ; Fc1c(-c2cccc3c2CCC3)ncc2c(N3CC4CCC(C3)N4)nc(OC[C@@]34CCCN3C[C@H](F)C4)nc12 ; CC1CCc2cccc(-c3ncc4c(N5CC6CCC(C5)N6)nc(OC[C@@]56CCCN5C[C@H](F)C6)nc4c3F)c21 ; Fc1ccc2cccc(-c3ncc4c(N5CC6CCC(C5)N6)nc(OC[C@@]56CCCN5C[C@H](F)C6)nc4c3F)c2c1F]

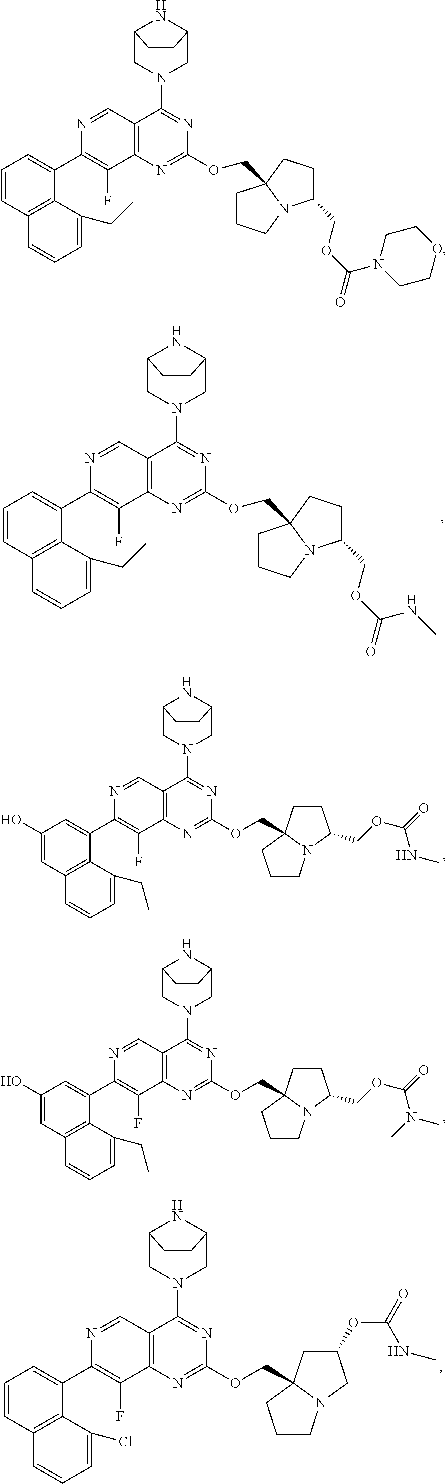 [SMILES: CCc1cccc2cccc(-c3ncc4c(N5CC6CCC(C5)N6)nc(OC[C@]56CCCN5[C@@H](COC(=O)N5CCOCC5)CC6)nc4c3F)c12 ; CCc1cccc2cccc(-c3ncc4c(N5CC6CCC(C5)N6)nc(OC[C@]56CCCN5[C@@H](COC(=O)NC)CC6)nc4c3F)c12 ; CCc1cccc2cc(O)cc(-c3ncc4c(N5CC6CCC(C5)N6)nc(OC[C@]56CCCN5[C@@H](COC(=O)NC)CC6)nc4c3F)c12 ; CCc1cccc2cc(O)cc(-c3ncc4c(N5CC6CCC(C5)N6)nc(OC[C@]56CCCN5[C@@H](COC(=O)N(C)C)CC6)nc4c3F)c12 ; CNC(=O)O[C@@H]1CN2CCC[C@]2(COc2nc(N3CC4CCC(C3)N4)c3cnc(-c4cccc5cccc(Cl)c45)c(F)c3n2)C1]

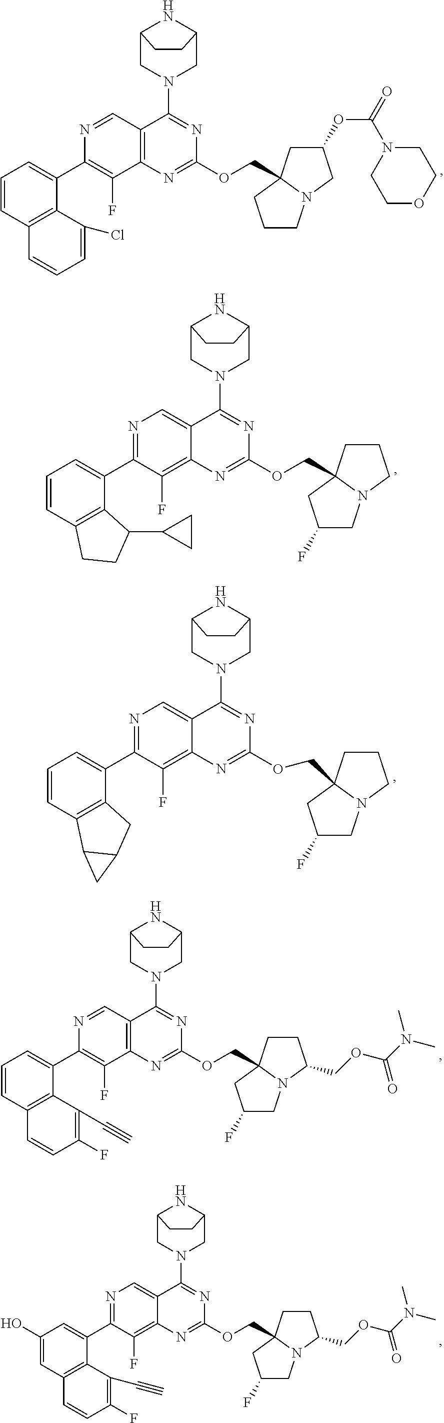 [SMILES: O=C(O[C@@H]1CN2CCC[C@]2(COc2nc(N3CC4CCC(C3)N4)c3cnc(-c4cccc5cccc(Cl)c45)c(F)c3n2)C1)N1CCOCC1 ; Fc1c(-c2cccc3c2C(C2CC2)CC3)ncc2c(N3CC4CCC(C3)N4)nc(OC[C@@]34CCCN3C[C@H](F)C4)nc12 ; Fc1c(-c2cccc3c2CC2CC32)ncc2c(N3CC4CCC(C3)N4)nc(OC[C@@]34CCCN3C[C@H](F)C4)nc12 ; C#Cc1c(F)ccc2cccc(-c3ncc4c(N5CC6CCC(C5)N6)nc(OC[C@@]56CC[C@H](COC(=O)N(C)C)N5C[C@H](F)C6)nc4c3F)c12 ; C#Cc1c(F)ccc2cc(O)cc(-c3ncc4c(N5CC6CCC(C5)N6)nc(OC[C@@]56CC[C@H](COC(=O)N(C)C)N5C[C@H](F)C6)nc4c3F)c12]

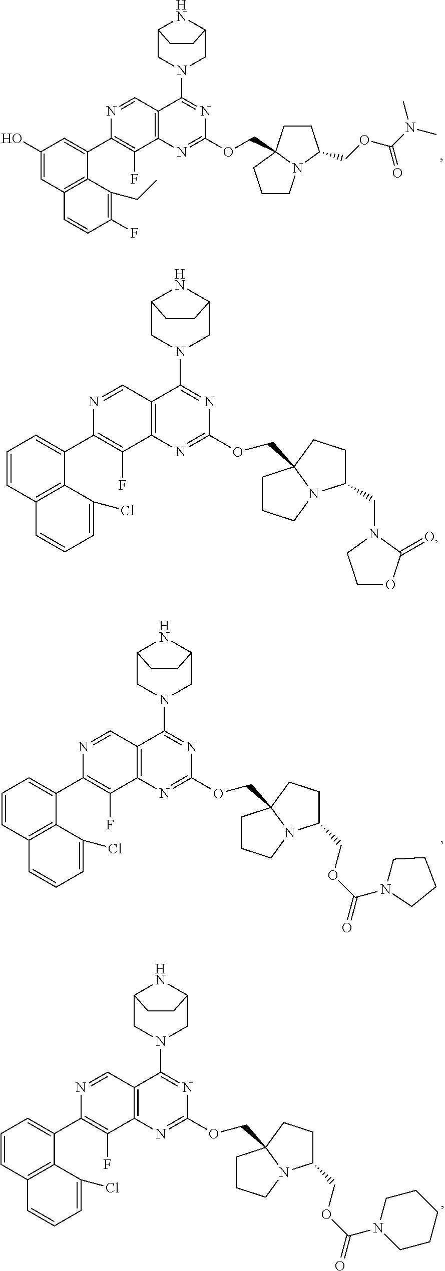 [SMILES: CCc1c(F)ccc2cc(O)cc(-c3ncc4c(N5CC6CCC(C5)N6)nc(OC[C@]56CCCN5[C@@H](COC(=O)N(C)C)CC6)nc4c3F)c12 ; O=C1OCCN1C[C@H]1CC[C@@]2(COc3nc(N4CC5CCC(C4)N5)c4cnc(-c5cccc6cccc(Cl)c56)c(F)c4n3)CCCN12 ; O=C(OC[C@H]1CC[C@@]2(COc3nc(N4CC5CCC(C4)N5)c4cnc(-c5cccc6cccc(Cl)c56)c(F)c4n3)CCCN12)N1CCCC1 ; O=C(OC[C@H]1CC[C@@]2(COc3nc(N4CC5CCC(C4)N5)c4cnc(-c5cccc6cccc(Cl)c56)c(F)c4n3)CCCN12)N1CCCCC1]

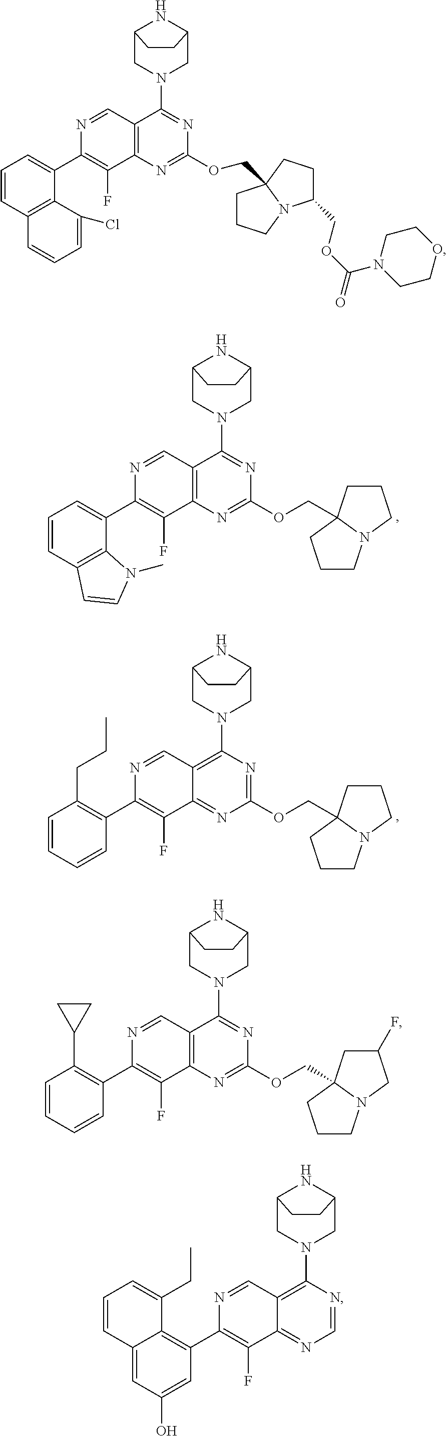 [SMILES: O=C(OC[C@H]1CC[C@@]2(COc3nc(N4CC5CCC(C4)N5)c4cnc(-c5cccc6cccc(Cl)c56)c(F)c4n3)CCCN12)N1CCOCC1 ; Cn1ccc2cccc(-c3ncc4c(N5CC6CCC(C5)N6)nc(OCC56CCCN5CCC6)nc4c3F)c21 ; CCCc1ccccc1-c1ncc2c(N3CC4CCC(C3)N4)nc(OCC34CCCN3CCC4)nc2c1F ; Fc1c(-c2ccccc2C2CC2)ncc2c(N3CC4CCC(C3)N4)nc(OC[C@@]34CCCN3CC(F)C4)nc12 ; CCc1cccc2cc(O)cc(-c3ncc4c(N5CC6CCC(C5)N6)ncnc4c3F)c12]

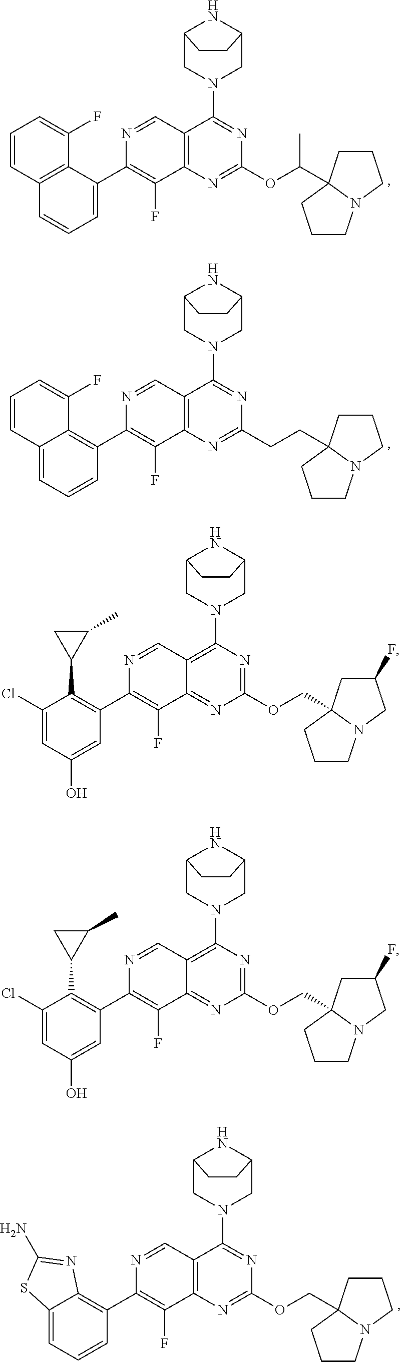 [SMILES: CC(Oc1nc(N2CC3CCC(C2)N3)c2cnc(-c3cccc4cccc(F)c34)c(F)c2n1)C12CCCN1CCC2 ; Fc1c(-c2cccc3cccc(F)c23)ncc2c(N3CC4CCC(C3)N4)nc(CCC34CCCN3CCC4)nc12 ; C[C@H]1C[C@@H]1c1c(Cl)cc(O)cc1-c1ncc2c(N3CC4CCC(C3)N4)nc(OC[C@@]34CCCN3C[C@H](F)C4)nc2c1F ; C[C@@H]1C[C@H]1c1c(Cl)cc(O)cc1-c1ncc2c(N3CC4CCC(C3)N4)nc(OC[C@@]34CCCN3C[C@H](F)C4)nc2c1F ; Nc1nc2c(-c3ncc4c(N5CC6CCC(C5)N6)nc(OCC56CCCN5CCC6)nc4c3F)cccc2s1]

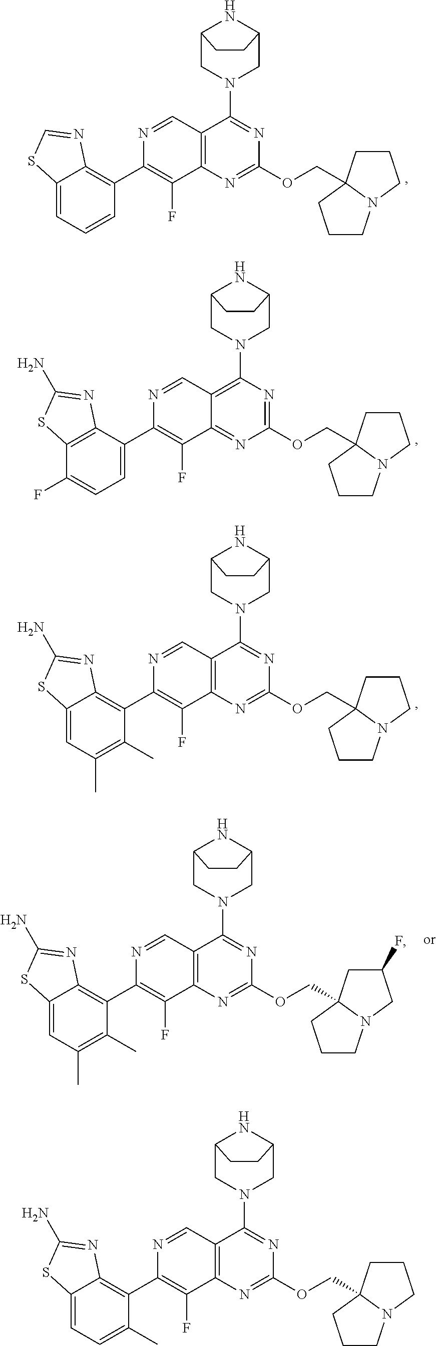

and pharmaceutically acceptable salts thereof. In one embodiment, the compounds of Formula (I) include bis-hydrochloride, tris-hydrochloride, trifluoroacetic acid, bis-trifluoroacetic acid, and tris-trifluoracetic acid salts of the above compounds. The compounds of Formula (I) or pharmaceutically acceptable salt thereof may be formulated into pharmaceutical compositions.

In certain embodiments of the present disclosure, the the KRAS^{G12D} inhibitor is

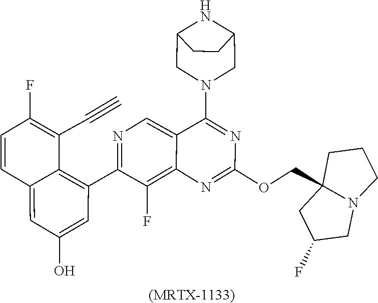

or a pharmaceutically acceptable salt thereof.

### PRMT5 Inhibitors

As provided above, the PRMT5 inhibitor is also administered in the methods of the disclosure. A “PRMT5 inhibitor” as used herein refers to compounds of the disclosure as described herein. These compounds are capable of negatively modulating or inhibiting all or a portion of the enzymatic activity of the PRMT5, particularly, in the presence of bound MTA in vitro or in vivo or in cells expressing elevated levels of MTA. In certain embodiments, the PRMT5 inhibitor is a MTA-cooperative PRMT5 inhibitor.

In certain embodiments, the PRMT5 inhibitor of the disclosure is any one of the PRMT5 inhibitors disclosed in International patent publication No. WO 2021/050915 A1, published 18 Mar. 2021, incorporated by reference in its entirety.

In certain other embodiments, the PRMT5 inhibitor of the disclosure is any one of the PRMT5 inhibitors disclosed International patent publication No. WO 2022/192745, published 15 Sep. 2022, incorporated by reference in its entirety.

In certain other embodiments, the PRMT5 inhibitor of the disclosure is any one of the PRMT5 inhibitors disclosed in International patent publication No. WO2023/081367, published 3 Aug. 2023, incorporated by reference in its entirety.

In certain other embodiments, the PRMT 5 inhibitor of the disclosure is any one of the PRMT5 inhibitors disclosed in International patent publication No. WO2023/278564, published 5 Jan. 2023, incorporated by reference in its entirety.

For example, the PRMT5 inhibitor in the methods of the disclosure as described herein is a compound of Formula IIA, IIB or IIC 1:

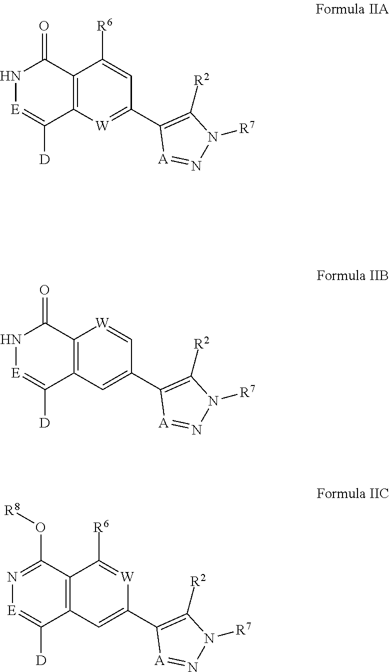

or a pharmaceutically acceptable salt thereof, wherein:
A is CR⁹ or N;
D is (C(R⁹)₂)₁₋₂—NH₂,

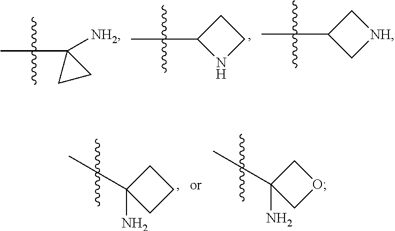 [SMILES: *C1(N)CC1 ; *C1CCN1 ; *C1CNC1 ; *C1(N)CCC1 ; *C1(N)COC1]

### or D is

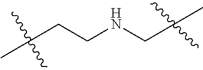 [SMILES: *CCNC*]

where the methylene is bonded to E where E is C;
E is C, CR⁹ or N;
each L is independently a bond or C1-C3 alkylene;
W is CR⁹ or N;
each X is independently a bond, O, S, —NR⁴— or —NR⁴C(O)—;
each Z is independently a bond, —SO—, —SO₂—, —CH(OH)— or —C(O)—;
each R² is independently hydroxy, halogen, cyano, cyanomethyl, —(NR⁴)₂, hydroxyalkyl, alkoxy, —SO₂C₁-C₃alkyl, -X-arC₁-C₃alkyl, heteroalkyl, C₂-C₄ alkynyl, -X-haloalkyl, -X-C₁-C₅ alkyl, -Z-C₁-C₅ alkyl, heterocyclyl, -X-L-cycloalkyl, -Z-cycloalkyl, -X-aryl, -Z-aryl, or -X-heteroaryl, wherein the heterocyclyl, the cycloalkyl, the aryl and the heteroaryl are optionally substituted with one or more R⁵.
each R⁴ is independently hydrogen or C₁-C₃ alkyl;
each R⁵ is independently cyano, oxo, halogen, C₁-C₃ alkyl, hydroxyalkyl, hydroxy, alkoxy, alkoxy-C₁-C₃ alkyl, -X-haloalkyl, -Z-cycloalkyl, -X-arC₁-C₃alkyl, -X-arC₁-C₃alkyl substituted with cyano, -X-L-cycloalkyl optionally substituted with C₁-C₃ alkyl or oxo, -X-L-heteroaryl optionally substituted with one or more C₁-C₃ alkyl or oxo, -X-L-heterocyclyl optionally substituted with one or more C₁-C₃ alkyl or oxo, or -X-aryl;
R⁶ is hydrogen, halogen, C₁-C₃ alkyl, haloalkyl, hydroxy, alkoxy, C₁-C₃ alkyl-alkoxy, N(R⁹)₂, NR⁹C(O)R⁹, C(O)R⁹, oxetane and THF;
R⁷ is H or C₁-C₃ alkyl optionally substituted with one or more halogen;
R⁸ is H or C₁-C₃ alkyl; and
each R⁹ is independently H or C₁-C₃ alkyl, halogen or haloalkyl.

Embodiment 2 provides the PRMT5 inhibitor in the methods of the disclosure as a compound of Formula IIA:

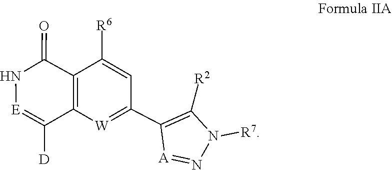

In certain embodiments, the PRMT5 inhibitor in the methods of the disclosure as a compound of Formula IIB:

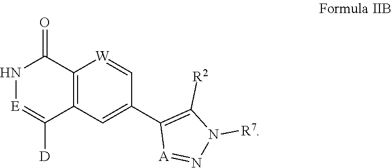

In certain embodiments, the PRMT5 inhibitor in the methods of the disclosure as a compound of Formula IIC:

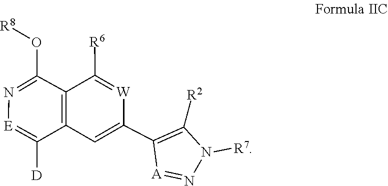

In certain embodiments of Formula IIA, IIB, and IIC, W is CR⁹.

In certain embodiments of Formula IIA, IIB, and IIC, A is CR⁹.

In certain embodiments of Formula IIA, IIB, and IIC, E is N.

In certain embodiments of Formula IIA, IIB, and IIC, W is CR⁹, A is CR⁹ and E is N.

Embodiment 9 provides the method of any of embodiments 1-8, wherein R² is selected from: benzothiophene, naphthalene, quinoline, chromane, isochromane, dihydrobenzodioxine, indolazine, tetrahydroindolazine, dihydroisobenzofuran, benzene, isoquinolinone, benzodioxone, thienopyridine, tetrahydroindolone, indolizine, dihydroindolizinone, imadazopyridinone, thienopyrimidine, thiophene, pyrrolopyrimidinone, thiazolopyridinone, dihydropyrrolizine, isoindalone and tetrahydroisoquinoline.

In certain embodiments of Formula IIA, IIB, and IIC, each R⁵ is independently cyano, oxo, halogen, C₁-C₃ alkyl, hydroxy, hydroxyalkyl, alkoxy-C₁-C₃alkyl, -X-L-heterocyclyl optionally substituted with one or more C₁-C₃alkyl or oxo, -X-L-cycloalkyl optionally substituted with C₁-C₃ alkyl or oxo.

In certain embodiments of Formula IIA, IIB, and IIC, R⁶ is selected from hydrogen, hydroxy, chlorine, —NHC(O)CH₃, —C(O)CF₂H, —NH₂, —CF₂, —CH₃, —O—CH₂CH₃, —CH₂—CH₂—O—CH₃, oxetane and THF.

In certain embodiments of Formula IIA, IIB, and IIC, one of L, X and Z is a bond. In certain embodiments, all of L, X and Z are bonds.

One aspect of the disclosure provides the method wherein the PRMT5 inhibitor is a compound of the Formula IIIC:

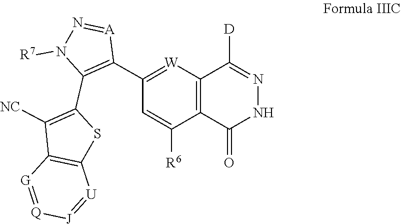

or a pharmaceutically acceptable salt thereof, wherein
A is CR⁹ or N;
D is —CH₂—NH₂,

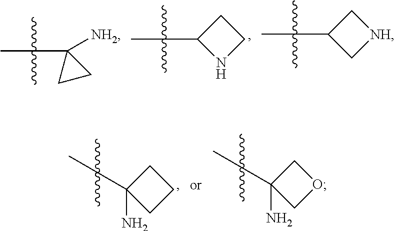 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
G, Q, J and U are independently selected from C(H), C(R⁵), and N, provided only one or two of G, Q, J, and U can be N;
   each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl;
R⁶ is hydrogen, halogen, C₁-C₆ alkyl, C₁-C₆ haloalkyl, hydroxy, C₁-C₆ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶,
   where each R⁹ is independently H or C₁-C₃ alkyl, R¹⁵ is hydrogen or methyl, and R¹⁶ is C₁-C₃ alkyl; and
R⁷ is C₁-C₃ alkyl or C₁-C₃ haloalkyl.

In certain embodiments of Formula IIIC, A is CH.

In certain embodiments of Formula IIIC, W is N.

In certain embodiments of Formula IIIC, W is CH.

In certain embodiments of Formula IIIC, D is —CH₂—NH₂.

In certain embodiments, the PRMT5 inhibitor is a compound according Formula IIIC having the formula:

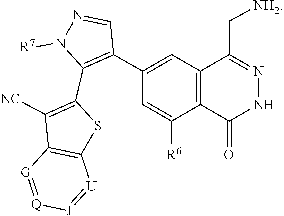

In certain embodiments of Formula IIIC, R⁶ is hydrogen, halogen, C₁-C₆ alkyl, C₁-C₆ haloalkyl, hydroxy, C₁-C₆ alkoxy, C₁-C₃ alkoxyC₁-C₃ alkyl, C₃-C₆ heterocycloalkyl, —C(O)-C₁-C₃ haloalkyl, —N(R⁹)₂, or —NR¹⁵(CO)R¹⁶.

In certain embodiments of Formula IIIC, R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

In certain embodiments of Formula IIIC, R⁶ is hydrogen, chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or -NH(CO)CH₃.

In certain embodiments of Formula IIIC, R⁶ is halogen, C₁-C₆ alkyl, C₁-C₆ haloalkyl, hydroxy, C₁-C₆ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

In certain embodiments of Formula IIIC, R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

In certain embodiments of Formula IIIC, R⁶ is chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

In certain embodiments of Formula IIIC, each G, Q, J and U is independently C(H).

In certain embodiments of Formula IIIC, G, Q, J and U are independently selected from C(H) and C(R⁵).

In certain embodiments of Formula IIIC, G, Q, J and U are independently selected from C(H) and N.

In certain embodiments of Formula IIIC,
R⁶ is hydrogen;
at least one of G, Q, J, and U is C(R⁵), and the remaining G, Q, J, and U are independently selected from C(H), C(R⁵) and N, wherein each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl.

For example, in certain embodiments, one or two of G, Q, J and U is N.

In certain embodiments of Formula IIIC,
R⁶ is hydrogen;
at least one of G, Q, J, and U is C(R⁵), and the remaining G, Q, J, and U are independently selected from C(H) and C(R⁵), wherein each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl.

For example, in certain embodiments, at least one of G, Q, J, and U is C(R⁵), and the remaining G, Q, J, and U are independently C(H); for example only one of G, Q, J, and U is C(R⁵). In certain embodiments, two of G, Q, J, and U is C(R⁵), and the remaining G, Q, J, and U are independently C(H). In certain embodiments, three of G, Q, J, and U is C(R⁵), and the remaining G, Q, J, and U is C(H).

In certain embodiments of Formula IIIC, G, Q, J, and U together with the thiophene to which they are attached form:

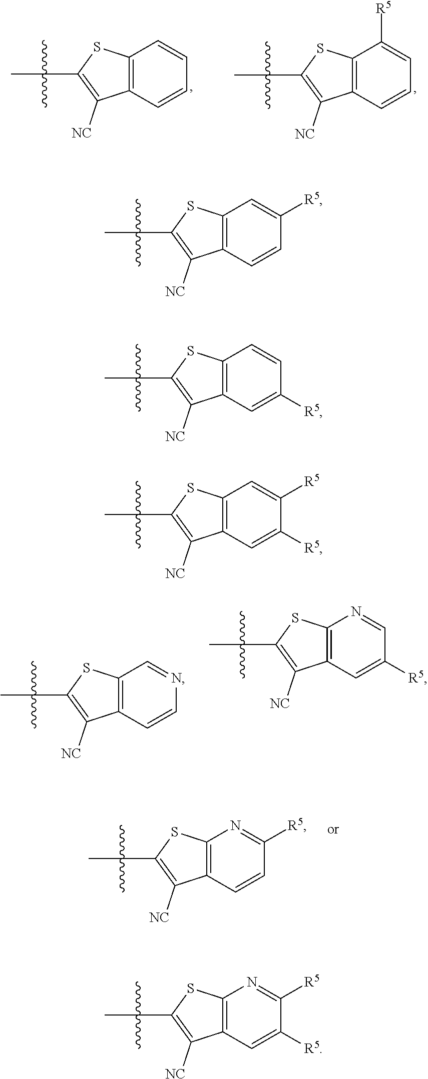

In certain embodiments of Formula IIIC, G, Q, J, and U together with the thiophene ring to which they are attached form a benzo[b]thiophene.

In certain embodiments of Formula IIIC, R⁵, if present, is hydroxy, halogen, C₁-C₃ alkyl, C₁-C₃ haloalkyl, C₁-C₃ alkoxy, C₁-C₃ haloalkoxy, C₃-C₆ cycloalkoxy, C₃-C₆ cycloalkyl, C₃-C₆ heterocycloalkyl, or C₁-C₃ alkoxyC₁-C₃ alkyl.

In certain embodiments of Formula IIIC, R⁵, if present, is hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl.

In certain embodiments of Formula IIIC, R⁵, if present, is hydroxy, chloro, fluoro, methyl, ethyl, methoxy, ethoxy, 2,2-difluoroethoxy, oxetanyl, tetrahydrofuranyl, (methoxy)methyl, (ethoxy)methyl, (methoxy)ethyl, or (ethoxy)ethyl.

In certain embodiments of Formula IIIC, R⁷ is methyl.

In certain embodiments of Formula IIIC, R⁷ is ethyl.

In certain embodiments of Formula IIIC, R⁷ is propyl (e.g., isopropyl).

In certain embodiments of Formula IIIC, R⁷ is difluoromethyl or trifluoromethyl.

In certain embodiments of Formula IIIC, the PRMT5 inhibitor is of the formula:

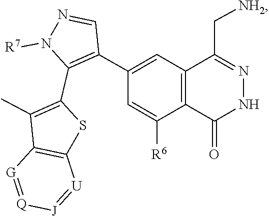

wherein
G, Q, J, and U together with the thiophene to which they are attached form:

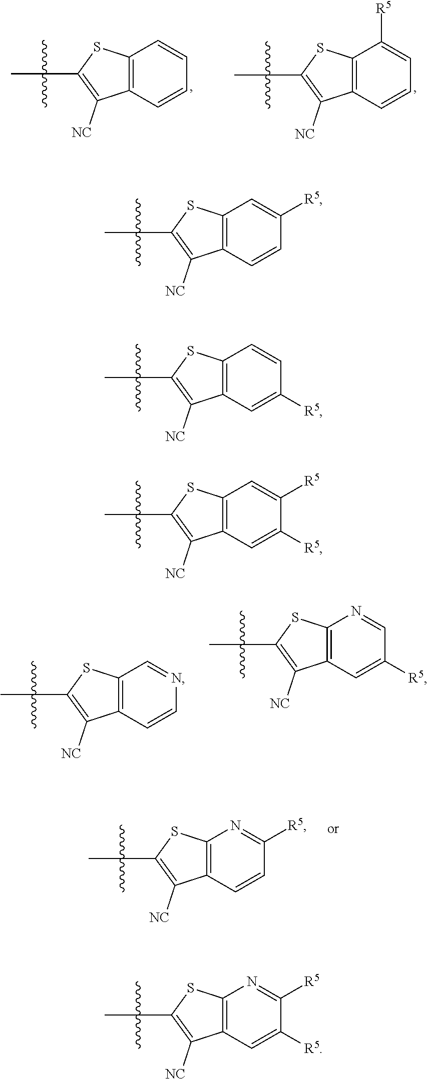

where each R⁵ is independently hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl; and
R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

In certain embodiments of Formula IIIC, the PRMT5 inhibitor is of the formula:

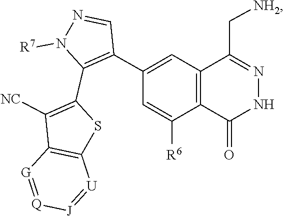

wherein
G, Q, J, and U together with the thiophene to which they are attached form:

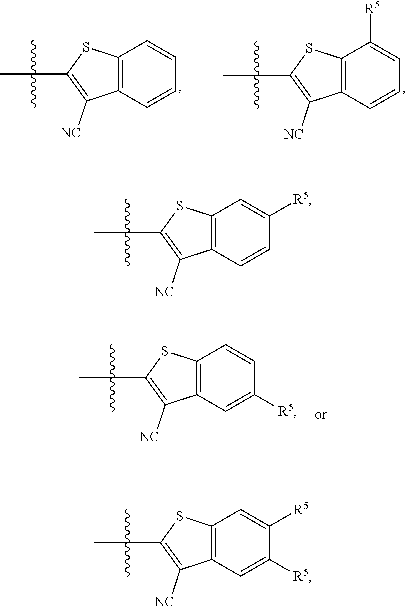

where each R⁵ is independently hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl; and
R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

In certain embodiments of Formula IIIC, the PRMT5 inhibitor is of the formula:

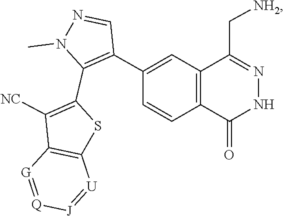

wherein
G, Q, J, and U together with the thiophene to which they are attached form:

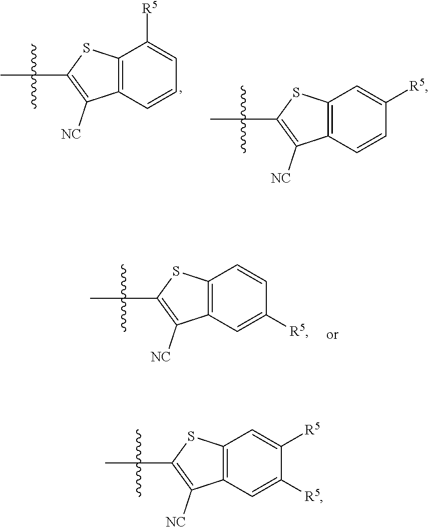

where each R⁵ is independently hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl.

In certain embodiments of the methods as described herein, the PRMT5 inhibitor is: the PRMT5 inhibitor is:

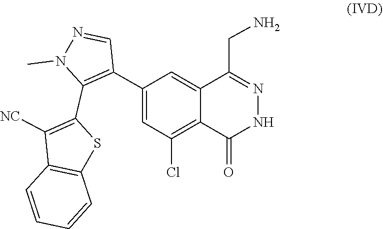 [SMILES: Cn1ncc(-c2cc(Cl)c3c(=O)[nH]nc(CN)c3c2)c1-c1sc2ccccc2c1C#N]

or a pharmaceutically acceptable salt thereof.

In certain embodiments, the PRMT5 inhibitor is a compound of the Formula IIIB:

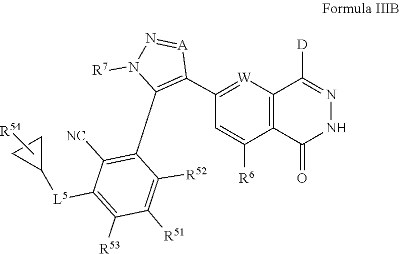

or a pharmaceutically acceptable salt thereof, wherein
A is CR⁹ or N;
D is —CH₂—NH₂,

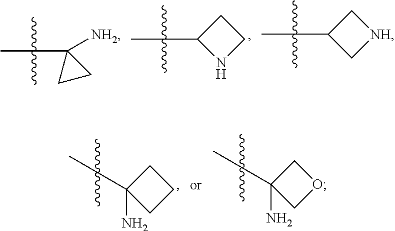 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
R⁵¹ is hydrogen, fluoro, chloro, or methyl, or R⁵¹ and R⁵² together with atoms to which they are attached form a C₄-C₆ heterocycloalkyl (e.g., hydrofuranyl);
R⁵² is fluoro, chloro, or methyl, or R⁵² and R⁵³ together with atoms to which they are attached form a phenyl;
R⁵³ is hydrogen, fluoro, chloro, or methyl;
R⁵⁴ is hydrogen, halogen, C₁-C₃ alkyl, or C₁-C₃ alkoxy;
L⁵ is —O— or —CH₂—;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵(CO)R¹⁶, where R¹⁵ is hydrogen or methyl, and R¹⁶ is C₁-C₃ alkyl;
R⁷ is C₁-C₃ alkyl or C₁-C₃ haloalkyl.

In certain embodiments of Formula IIIB:
A is —CH or —CCH₃;
D is —CH₂—NH₂;
W is —CH, —CCH₃, or N;
R⁵¹, R⁵², R⁵³, and R⁵⁴ are each independently selected from hydrogen, fluoro, chloro, or methyl;
L⁵ is —O—;
R⁶ is hydrogen, fluoro, chloro, or methyl; and
R⁷ is C₁-C₂ alkyl or C₁-C₂ haloalkyl.

In certain embodiments of Formula IIIB:
A and W are —CH;
D is —CH₂—NH₂;
R⁵¹, R⁵², and R⁵³ are each independently selected from hydrogen, fluoro, chloro, and methyl;
R⁵⁴ is hydrogen;
L⁵ is —O—;
R⁶ is hydrogen; and
R⁷ is methyl.

In certain embodiments of Formula IIIB:
A and W are —CH;
D is —CH₂—NH₂;
R⁵¹ and R⁵² are each independently selected from fluoro, chloro, and methyl;
R⁵³ and R⁵4 are hydrogen;
L⁵ is —O—;
R⁶ is hydrogen; and
R⁷ is methyl.

In certain embodiments of Formula IIIB, A is CH.

In certain embodiments of Formula IIIB, W is N.

In certain embodiments of Formula IIIB, W is CH.

In certain embodiments of Formula IIIB, D is —CH₂—NH₂.

In certain embodiments of Formula IIIB, R⁵⁴ is hydrogen or methyl.

In certain embodiments of Formula IIIB, R⁵⁴ is hydrogen.

In certain embodiments of Formula IIIB, R⁵⁴ is methyl.

In certain embodiments of Formula IIIB, the PRMT5 inhibitor is of the formula:

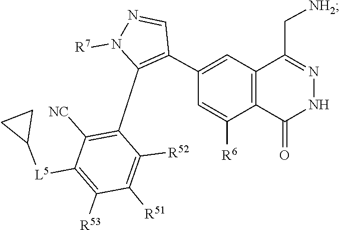

such as e.g.,

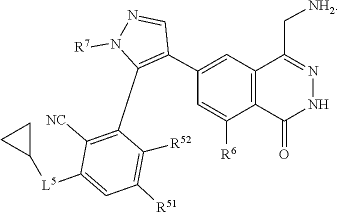

In certain embodiments of Formula IIIB, L⁵ is —CH₂—.

In certain embodiments of Formula IIIB, L⁵ is —O—.

In certain embodiments of Formula IIIB, R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is hydrogen, chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)— difluoromethyl, —NH₂, or —NH(CO)CH₃.

In certain embodiments of Formula IIIB, R⁶ is hydrogen, halogen, C₁-C₆ alkyl, or C₁-C₆ alkoxy; for example, R⁶ is hydrogen, halogen, C₁-C₃ alkyl, or C₁-C₃ alkoxy.

In certain embodiments of Formula IIIB, R⁶ is hydrogen, chloro, fluoro, methyl, ethyl, methoxy, or ethoxy.

In certain embodiments of Formula IIIB, R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or -NH(CO)CH₃.

In certain embodiments of Formula IIIB, R⁶ is halogen, C₁-C₆ alkyl, or C₁-C₆ alkoxy; for example, R⁶ is halogen, C₁-C₃ alkyl, or C₁-C₃ alkoxy.

In certain embodiments of Formula IIIB, R⁶ is chloro, fluoro, methyl, ethyl, methoxy, or ethoxy.

In certain embodiments of Formula IIIB, R⁷ is methyl.

In certain embodiments of Formula IIIB, R⁷ is ethyl.

In certain embodiments of Formula IIIB, R⁷ is propyl (e.g., isopropyl).

In certain embodiments of Formula IIIB, R⁷ is difluoromethyl or trifluoromethyl.

In certain embodiments of Formula IIIB, R⁵³ is hydrogen or methoxy; or wherein R⁵³ is hydrogen.

In certain embodiments of Formula IIIB, the PRMT5 inhibitor is of the formula:

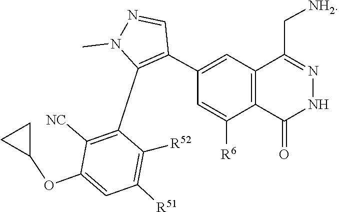

In certain embodiments, R⁵² is fluoro, and R⁵¹ is hydrogen, fluoro, chloro, or methyl.

In certain embodiments of Formula IIIB, R⁵² is fluoro, and R⁵¹ is chloro.

In certain embodiments of Formula IIIB, R⁵² is fluoro, and R⁵¹ is methyl or hydrogen (for example, R⁵² is fluoro and R⁵¹ is methyl; or R⁵² is fluoro and R⁵¹ is hydrogen).

In certain embodiments of Formula IIIB, R⁵¹ and R⁵² together with atoms to which they are attached form a hydrofuranyl

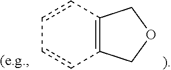

In certain embodiments of Formula IIIB, the PRMT5 inhibitor is

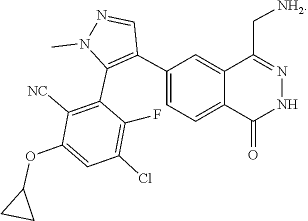 [SMILES: Cn1ncc(-c2ccc3c(=O)[nH]nc(CN)c3c2)c1-c1c(F)c(Cl)cc(OC2CC2)c1C#N]

In certain embodiments of Formula IIIB, the PRMT5 inhibitor is

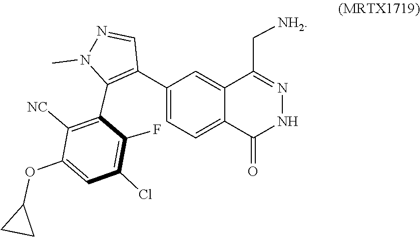

One aspect of the disclosure provides the method wherein the PRMT5 inhibitor is a compound of the Formula IIIA:

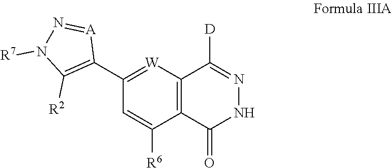

or a pharmaceutically acceptable salt thereof, wherein
A is CR⁹ or N;
D is —CH₂—NH₂,

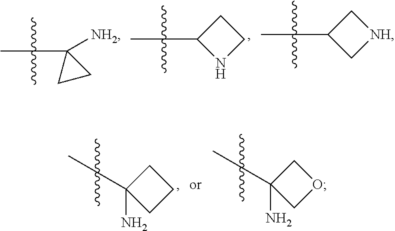 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
R² is

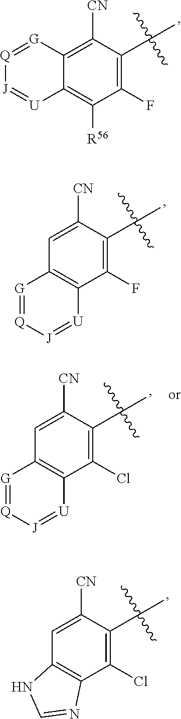

where R⁵⁶ is hydrogen, fluoro, chloro, or methyl,
G, Q, J and U are independently selected from C(H), C(R⁵), and N, provided only one or two of G, Q, J, and U can be N;
   each R⁵ is independently hydroxy, halogen, C₁-C₆ alkyl, C₁-C₆ haloalkyl, C₁-C₆alkoxy, C₁-C₆ haloalkoxy, C₃-C₆ cycloalkoxy, C₃-C₆ cycloalkyl, C₃-C₆heterocycloalkyl, or C₁-C₃ alkoxyC₁-C₃ alkyl;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵(CO)R¹⁶, where R¹⁵ is hydrogen or methyl, and R¹⁶ is C₁-C₃ alkyl; and
R⁷ is C₁-C₃ alkyl or C₁-C₃ haloalkyl.

One aspect of the disclosure provides the method wherein the PRMT5 inhibitor is a compound of the Formula IIIA:

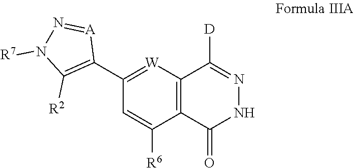

or a pharmaceutically acceptable salt thereof, wherein
A is CR⁹ or N;
D is —CH₂—NH₂,

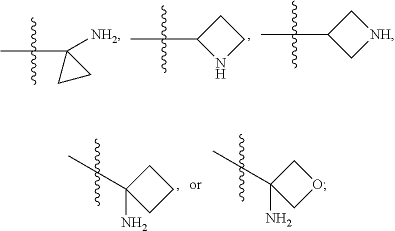 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
R² is

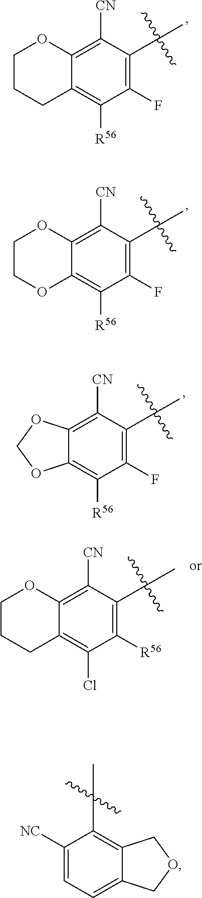

where R⁵⁶ is hydrogen, halogen, C₁-C₆ alkyl, C₁-C₆ haloalkyl, C₁-C₆ alkoxy, or C₁-C₆ haloalkoxy;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵(CO)R¹⁶, where R¹⁵ is hydrogen or methyl, and R¹⁶ is C₁-C₃ alkyl; and
R⁷ is C₁-C₃ alkyl or C₁-C₃ haloalkyl.

In certain embodiments of Formula IIIA, A is CH.

In certain embodiments of Formula IIIA, W is N.

In certain embodiments of Formula IIIA, W is CH.

In certain embodiments of Formula IIIA, D is —CH₂—NH₂.

In certain embodiments of Formula IIIA, the PRMT5 inhibitor is of the formula:

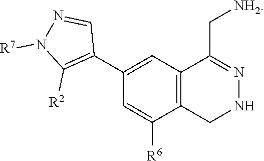

In certain embodiments of Formula IIIA, R² is

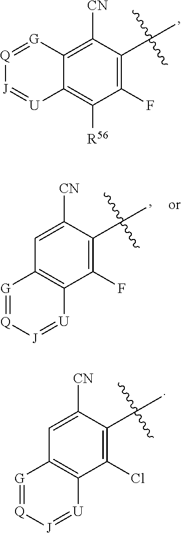

In certain embodiments of Formula IIIA, G, Q, J and U are independently selected from C(H) and C(R⁵).

In certain embodiments of Formula IIIA, G, Q, J and U are independently C(H).

In certain embodiments of Formula IIIA, at least one of G, Q, J, and U is C(R⁵), and the remaining G, Q, J, and U are independently C(H); for example only one of G, Q, J, and U is C(R⁵).

In certain embodiments of Formula IIIA, U is N, and G, Q, and J are independently selected from C(H) and C(R⁵).

In certain embodiments of Formula IIIA, G is N, and Q, J, and U are independently selected from C(H) and C(R⁵).

In certain embodiments of Formula IIIA, R⁵, if present, is hydroxy, halogen, C₁-C₃ alkyl, C₁-C₃ haloalkyl, C₁-C₃ alkoxy, C₁-C₃ haloalkoxy, C₃-C₆ cycloalkoxy, C₃-C₆ cycloalkyl, C₃-C₆ heterocycloalkyl, or C₁-C₃ alkoxyC₁-C₃ alkyl.

In certain embodiments of Formula IIIA, R⁵, if present, is hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl.

In certain embodiments of Formula IIIA, R⁵, if present, is hydroxy, chloro, fluoro, methyl, ethyl, methoxy, ethoxy, 2,2-difluoroethoxy, oxetanyl, tetrahydrofuranyl, (methoxy)methyl, (ethoxy)methyl, (methoxy)ethyl, or (ethoxy)ethyl.

In certain embodiments of Formula IIIA, R⁵, if present, is halogen, C₁-C₆ alkyl, or C₁-C₆ alkoxy; for example, R⁶ is halogen, C₁-C₃ alkyl, or C₁-C₃ alkoxy.

In certain embodiments of Formula IIIA, R⁵, if present, is chloro, fluoro, methyl, ethyl, methoxy, or ethoxy.

In certain embodiments of Formula IIIA, R⁵⁶ is fluoro, chloro, or methyl.

In certain embodiments of Formula IIIA, R² is

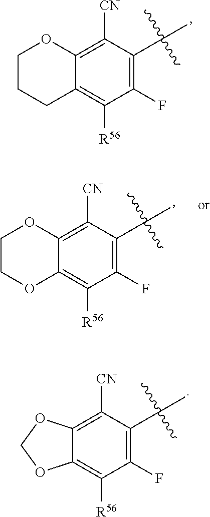

In certain embodiments of Formula IIIA, R⁵⁶ is hydrogen, fluoro, chloro, or methyl.

In certain embodiments of Formula IIIA, R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is hydrogen, chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)— difluoromethyl, —NH₂, or —NH(CO)CH₃.

In certain embodiments of Formula IIIA, R⁶ is hydrogen, halogen, C₁-C₆ alkyl, or C₁-C₆ alkoxy; for example, R⁶ is hydrogen, halogen, C₁-C₃ alkyl, or C₁-C₃ alkoxy.

In certain embodiments of Formula IIIA, R⁶ is hydrogen, chloro, fluoro, methyl, ethyl, methoxy, or ethoxy.

In certain embodiments of Formula IIIA, R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or -NH(CO)CH₃.

In certain embodiments of Formula IIIA, R⁶ is halogen, C₁-C₆ alkyl, or C₁-C₆ alkoxy; for example, R⁶ is halogen, C₁-C₃ alkyl, or C₁-C₃ alkoxy.

In certain embodiments of Formula IIIA, R⁶ is chloro, fluoro, methyl, ethyl, methoxy, or ethoxy.

In certain embodiments of Formula IIIA, R⁷ is methyl.

In certain embodiments of Formula IIIA, R⁷ is ethyl.

In certain embodiments of Formula IIIA, R⁷ is propyl (e.g., isopropyl).

In certain embodiments of Formula IIIA, R⁷ is difluoromethyl or trifluoromethyl.

In certain embodiments of the methods of the disclosure as described herein, the PRMT5 inhibitor is:

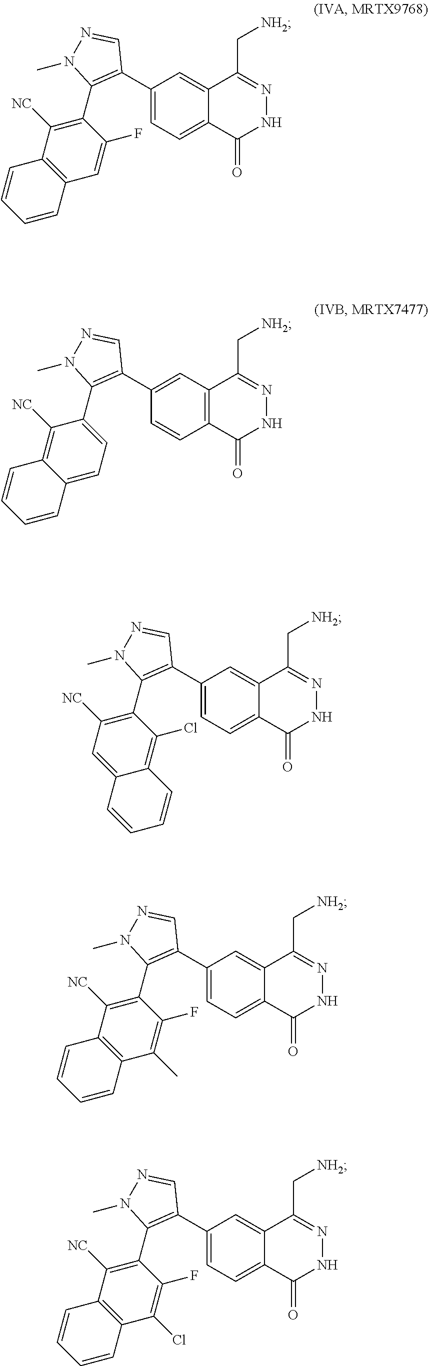 [SMILES: Cn1ncc(-c2ccc3c(=O)[nH]nc(CN)c3c2)c1-c1c(F)cc2ccccc2c1C#N ; Cn1ncc(-c2ccc3c(=O)[nH]nc(CN)c3c2)c1-c1ccc2ccccc2c1C#N ; Cn1ncc(-c2ccc3c(=O)[nH]nc(CN)c3c2)c1-c1c(C#N)cc2ccccc2c1Cl ; Cc1c(F)c(-c2c(-c3ccc4c(=O)[nH]nc(CN)c4c3)cnn2C)c(C#N)c2ccccc12 ; Cn1ncc(-c2ccc3c(=O)[nH]nc(CN)c3c2)c1-c1c(F)c(Cl)c2ccccc2c1C#N]

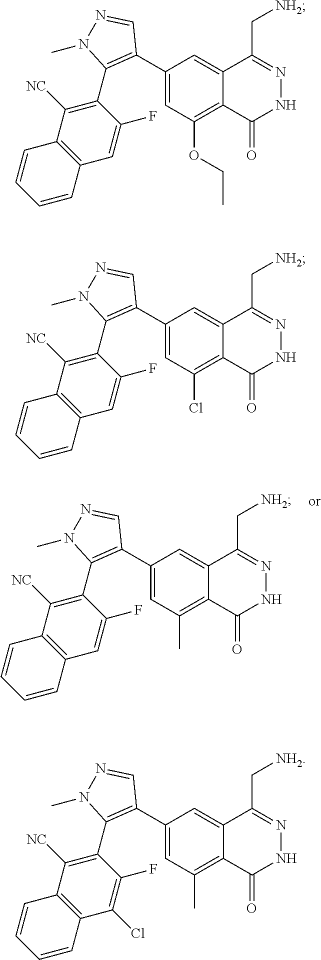 [SMILES: CCOc1cc(-c2cnn(C)c2-c2c(F)cc3ccccc3c2C#N)cc2c(CN)n[nH]c(=O)c12 ; Cn1ncc(-c2cc(Cl)c3c(=O)[nH]nc(CN)c3c2)c1-c1c(F)cc2ccccc2c1C#N ; Cc1cc(-c2cnn(C)c2-c2c(F)cc3ccccc3c2C#N)cc2c(CN)n[nH]c(=O)c12 ; Cc1cc(-c2cnn(C)c2-c2c(F)c(Cl)c3ccccc3c2C#N)cc2c(CN)n[nH]c(=O)c12]

In certain embodiments of the methods of the disclosure as described herein, the PRMT5 inhibitor is:

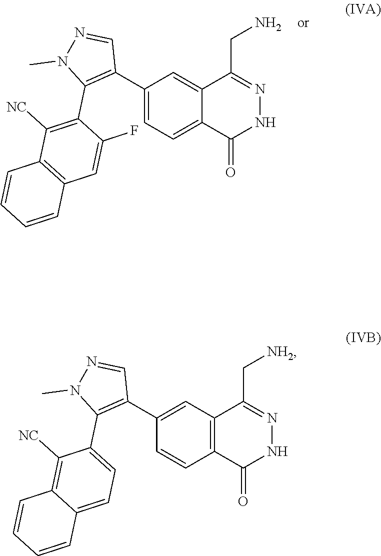 [SMILES: Cn1ncc(-c2ccc3c(=O)[nH]nc(CN)c3c2)c1-c1c(F)cc2ccccc2c1C#N ; Cn1ncc(-c2ccc3c(=O)[nH]nc(CN)c3c2)c1-c1ccc2ccccc2c1C#N]

or a pharmaceutically acceptable salt thereof.

In certain embodiments of the methods of the disclosure as described herein, the PRMT5 inhibitor is:

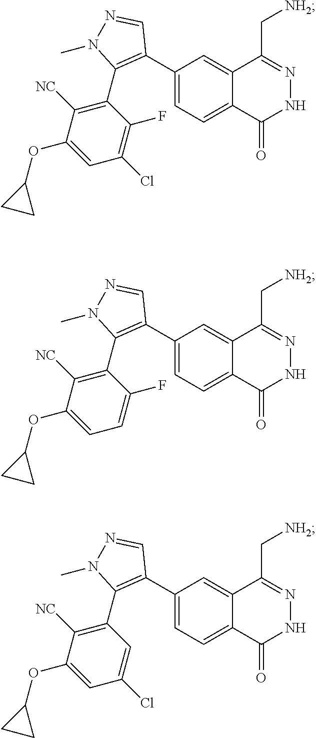 [SMILES: Cn1ncc(-c2ccc3c(=O)[nH]nc(CN)c3c2)c1-c1c(F)c(Cl)cc(OC2CC2)c1C#N ; Cn1ncc(-c2ccc3c(=O)[nH]nc(CN)c3c2)c1-c1c(F)ccc(OC2CC2)c1C#N ; Cn1ncc(-c2ccc3c(=O)[nH]nc(CN)c3c2)c1-c1cc(Cl)cc(OC2CC2)c1C#N]

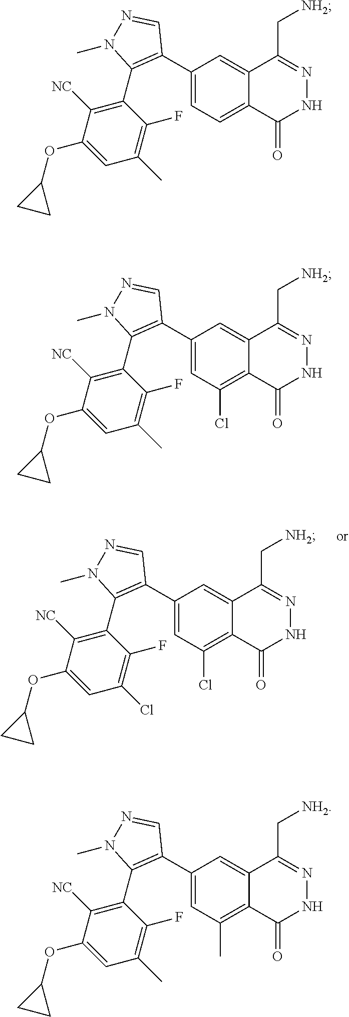 [SMILES: Cc1cc(OC2CC2)c(C#N)c(-c2c(-c3ccc4c(=O)[nH]nc(CN)c4c3)cnn2C)c1F ; Cc1cc(OC2CC2)c(C#N)c(-c2c(-c3cc(Cl)c4c(=O)[nH]nc(CN)c4c3)cnn2C)c1F ; Cn1ncc(-c2cc(Cl)c3c(=O)[nH]nc(CN)c3c2)c1-c1c(F)c(Cl)cc(OC2CC2)c1C#N ; Cc1cc(OC2CC2)c(C#N)c(-c2c(-c3cc(C)c4c(=O)[nH]nc(CN)c4c3)cnn2C)c1F]

In certain embodiments of the methods of the disclosure as described herein, the PRMT5 inhibitor is:

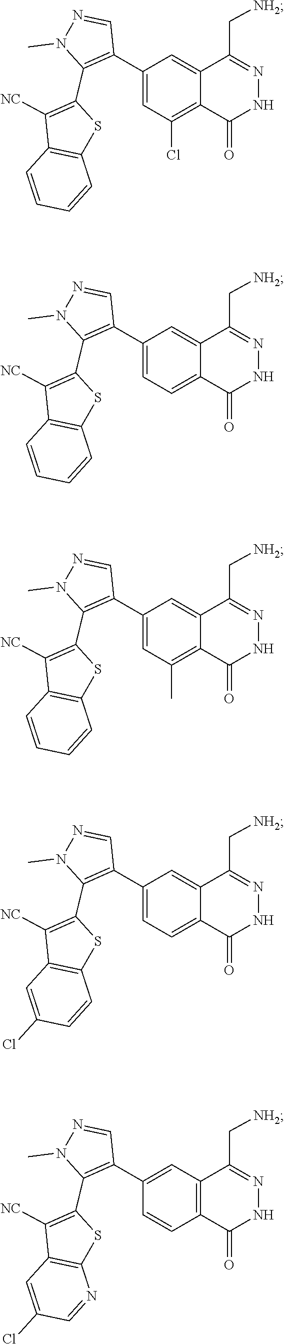 [SMILES: Cn1ncc(-c2cc(Cl)c3c(=O)[nH]nc(CN)c3c2)c1-c1sc2ccccc2c1C#N ; Cn1ncc(-c2ccc3c(=O)[nH]nc(CN)c3c2)c1-c1sc2ccccc2c1C#N ; Cc1cc(-c2cnn(C)c2-c2sc3ccccc3c2C#N)cc2c(CN)n[nH]c(=O)c12 ; Cn1ncc(-c2ccc3c(=O)[nH]nc(CN)c3c2)c1-c1sc2ccc(Cl)cc2c1C#N ; Cn1ncc(-c2ccc3c(=O)[nH]nc(CN)c3c2)c1-c1sc2ncc(Cl)cc2c1C#N]

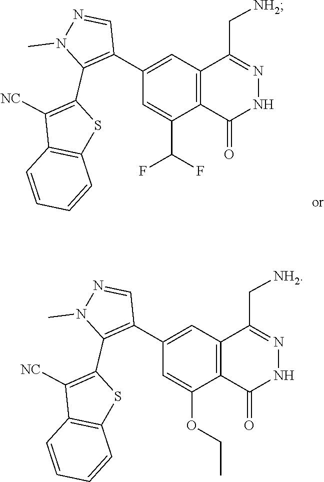 [SMILES: Cn1ncc(-c2cc(C(F)F)c3c(=O)[nH]nc(CN)c3c2)c1-c1sc2ccccc2c1C#N ; CCOc1cc(-c2cnn(C)c2-c2sc3ccccc3c2C#N)cc2c(CN)n[nH]c(=O)c12]

In certain embodiments of the methods of the disclosure as described herein, the PRMT5 inhibitor is:

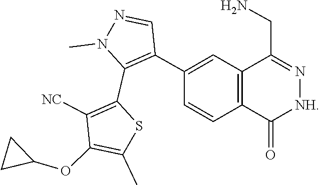 [SMILES: Cc1sc(-c2c(-c3ccc4c(=O)[nH]nc(CN)c4c3)cnn2C)c(C#N)c1OC1CC1]

In certain embodiments as described herein, the PRMT5 inhibitor is

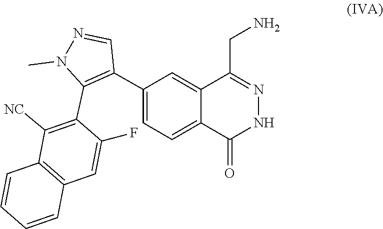 [SMILES: Cn1ncc(-c2ccc3c(=O)[nH]nc(CN)c3c2)c1-c1c(F)cc2ccccc2c1C#N]

or a pharmaceutically acceptable salt, and the KRAS^{G12D} inhibitor is compound of Formula I or a pharmaceutically acceptable salt thereof. For example, in some embodiments, the PRMT5 inhibitor is (IVA)

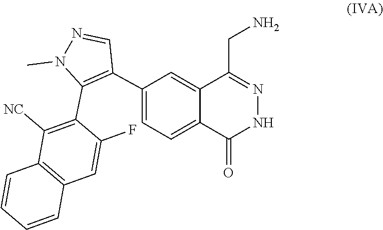 [SMILES: Cn1ncc(-c2ccc3c(=O)[nH]nc(CN)c3c2)c1-c1c(F)cc2ccccc2c1C#N]

or a pharmaceutically acceptable salt, and the KRAS^{G12D} inhibitor is MRTX-1133 or a pharmaceutically acceptable salt thereof.

In certain embodiments as described herein, the PRMT5 inhibitor is

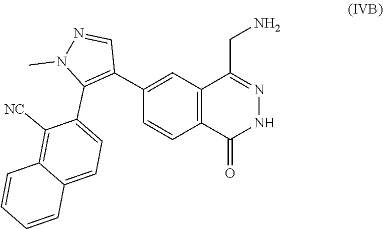 [SMILES: Cn1ncc(-c2ccc3c(=O)[nH]nc(CN)c3c2)c1-c1ccc2ccccc2c1C#N]

or a pharmaceutically acceptable salt, and the KRAS^{G12D} inhibitor is compound of Formula I or a pharmaceutically acceptable salt thereof. For example, in some embodiments, the PRMT5 inhibitor is (IVB)

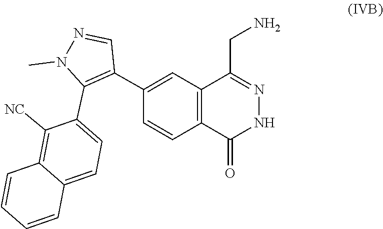 [SMILES: Cn1ncc(-c2ccc3c(=O)[nH]nc(CN)c3c2)c1-c1ccc2ccccc2c1C#N]

or a pharmaceutically acceptable salt, and the KRAS^{G12D} inhibitor is MRTX-1133 or a pharmaceutically acceptable salt thereof.

In certain embodiments as described herein, the PRMT5 inhibitor is

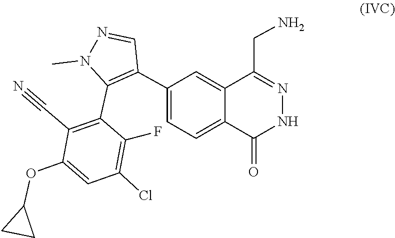 [SMILES: Cn1ncc(-c2ccc3c(=O)[nH]nc(CN)c3c2)c1-c1c(F)c(Cl)cc(OC2CC2)c1C#N]

or a pharmaceutically acceptable salt thereof, and the KRAS^{G12D} inhibitor is a compound of Formula 1 or a pharmaceutically acceptable salt thereof. For example, in some embodiments, the PRMT5 inhibitor is

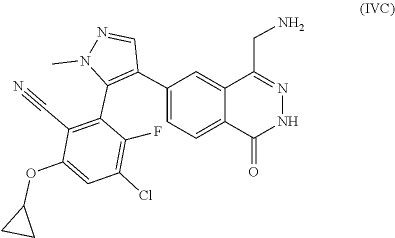 [SMILES: Cn1ncc(-c2ccc3c(=O)[nH]nc(CN)c3c2)c1-c1c(F)c(Cl)cc(OC2CC2)c1C#N]

or a pharmaceutically acceptable salt thereof, and the KRAS^{G12D} inhibitor is MRTX-1133 or a pharmaceutically acceptable salt thereof.

In some embodiments as described herein, the PRMT5 inhibitor is MRTX1719 or a pharmaceutically acceptable salt thereof, and the KRAS^{G12D} inhibitor is MRTX-1133 or a pharmaceutically acceptable salt thereof.

In some embodiments as described herein, the PRMT5 inhibitor is:

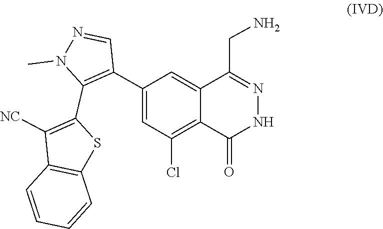 [SMILES: Cn1ncc(-c2cc(Cl)c3c(=O)[nH]nc(CN)c3c2)c1-c1sc2ccccc2c1C#N]

or a pharmaceutically acceptable salt thereof, and the KRAS^{G12D} inhibitor is a compound of Formula 1 or a pharmaceutically acceptable salt thereof. For example, in some embodiments as described herein, the PRMT5 inhibitor is:

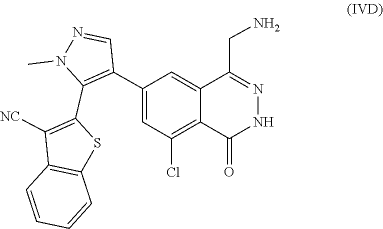 [SMILES: Cn1ncc(-c2cc(Cl)c3c(=O)[nH]nc(CN)c3c2)c1-c1sc2ccccc2c1C#N]

or a pharmaceutically acceptable salt thereof, and the KRAS^{G12D} inhibitor is MRTX-1133 or a pharmaceutically acceptable salt thereof.

### Definitions

For simplicity, chemical moieties are defined and referred to throughout primarily as univalent chemical moieties (e.g., alkyl, aryl, etc.). Nevertheless, such terms may also be used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for 0, and 2, 4, or 6 for S, depending on the oxidation state of the S).

The term “amino” refers to —NH₂.

The term “acetyl” refers to “—C(O)CH₃.

As herein employed, the term “acyl” refers to an alkylcarbonyl or arylcarbonyl substituent wherein the alkyl and aryl portions are as defined herein.

The term “alkyl” as employed herein refers to saturated straight and branched chain aliphatic groups having from 1 to 12 carbon atoms. As such, “alkyl” encompasses C₁, C₂, C₃, C₄, C₅, C₆, C₇, C₈, C₉, C₁₀, C₁₁ and C₁₂ groups. Examples of alkyl groups include, without limitation, methyl, ethyl, propyl, isopropyl, butyl, isobutyl, sec-butyl, tert-butyl, pentyl, and hexyl.

The term “alkenyl” as used herein means an unsaturated straight or branched chain aliphatic group with one or more carbon-carbon double bonds, having from 2 to 12 carbon atoms. As such, “alkenyl” encompasses C₂, C₃, C₄, C₅, C₆, C₇, C₈, C₉, C₁₀, C₁₁ and C₁₂ groups.

Examples of alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms. As such, “alkynyl” encompasses C₂, C₃, C₄, C₅, C₆, C₇, C₈, C₉, C₁₀, C₁₁ and C₁₂ groups.

Examples of alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect two other chemical groups. Examples of alkylene groups include, without limitation, methylene, ethylene, propylene, and butylene. Exemplary alkenylene groups include, without limitation, ethenylene, propenylene, and butenylene. Exemplary alkynylene groups include, without limitation, ethynylene, propynylene, and butynylene.

The term “alkoxy” refers to —OC₁-C₆ alkyl.

The term “cycloalkyl” as employed herein is a saturated and partially unsaturated cyclic hydrocarbon group having 3 to 12 carbons. As such, “cycloalkyl” includes C₃, C₄, C₅, C₆, C₇, C₈, C₉, C₁₀, C₁₁ and C₁₂ cyclic hydrocarbon groups. Examples of cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” refers to an alkyl group, as defined hereinabove, wherein one or more carbon atoms in the chain are independently replaced 0, S, or NRX, wherein R^{X} is hydrogen or C₁-C₃ alkyl. Examples of heteroalkyl groups include methoxymethyl, methoxyethyl and methoxypropyl.

An “aryl” group is a C₆-C₁₄ aromatic moiety comprising one to three aromatic rings. As such, “aryl” includes C₆, C₁₀, C₁₃, and C₁₄ cyclic hydrocarbon groups. An exemplary aryl group is a C₆-C₁₀ aryl group. Particular aryl groups include, without limitation, phenyl, naphthyl, anthracenyl, and fluorenyl. An “aryl” group also includes fused multicyclic (e.g., bicyclic) ring systems in which one or more of the fused rings is non-aromatic, provided that at least one ring is aromatic, such as indenyl.

An “aralkyl” or “arylalkyl” group comprises an aryl group covalently linked to an alkyl group wherein the moiety is linked to another group via the alkyl moiety. An exemplary aralkyl group is —(C₁-C₆)alkyl(C₆-C₁₀)aryl, including, without limitation, benzyl, phenethyl, and naphthylmethyl. For example, an arC₁-C₃alkyl is an aryl group covalently linked to a C₁-C₃ alkyl.

A “heterocyclyl” or “heterocyclic” group is a mono- or bicyclic (fused or spiro) ring structure having from 3 to 12 atoms, (3, 4, 5, 6, 7, 8, 9, 10, 11 or 12 atoms), for example 4 to 8 atoms, wherein one or more ring atoms are independently —C(O)—, N, NR⁴, O, or S, and the remainder of the ring atoms are quaternary or carbonyl carbons. Examples of heterocyclic groups include, without limitation, epoxy, oxiranyl, oxetanyl, azetidinyl, aziridinyl, tetrahydrofuranyl, tetrahydropyranyl, tetrahydrothiophenyl, pyrrolidinyl, piperidinyl, piperazinyl, imidazolidinyl, thiazolidinyl, thiatanyl, dithianyl, trithianyl, azathianyl, oxathianyl, dioxolanyl, oxazolidinyl, oxazolidinonyl, decahydroquinolinyl, piperidonyl, 4-piperidonyl, thiomorpholinyl, dimethyl-morpholinyl, and morpholinyl. Specifically excluded from the scope of this term are compounds having adjacent ring O and/or S atoms.

As used herein, “L-heterocyclyl” refers to a heterocyclyl group covalently linked to another group via an alkylene linker.

As used herein, the term “heteroaryl” refers to a group having 5 to 14 ring atoms, preferably 5, 6, 10, 13 or 14 ring atoms; having 6, 10, or 14 π electrons shared in a cyclic array; and having, in addition to carbon atoms, from one to three heteroatoms that are each independently N, O, or S. Heteroaryl also includes fused multicyclic (e.g., bicyclic) ring systems in which one or more of the fused rings is non-aromatic, provided that at least one ring is aromatic and at least one ring contains an N, O, or S ring atom. Examples of heteroaryl groups include acridinyl, azocinyl, benzimidazolyl, benzofuranyl, benzo[d]oxazol-2(3H)-one, 2H-benzo[b][1,4]oxazin-3(4H)-one, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

A “L-heteroaralkyl” or “L-heteroarylalkyl” group comprises a heteroaryl group covalently linked to another group via an alkylene linker. Examples of heteroalkyl groups comprise a C₁-C₆ alkyl group and a heteroaryl group having 5, 6, 9, or 10 ring atoms. Examples of heteroaralkyl groups include pyridylmethyl, pyridylethyl, pyrrolylmethyl, pyrrolylethyl, imidazolylmethyl, imidazolylethyl, thiazolylmethyl, thiazolylethyl, benzimidazolylmethyl, benzimidazolylethyl quinazolinylmethyl, quinolinylmethyl, quinolinylethyl, benzofuranylmethyl, indolinylethyl isoquinolinylmethyl, isoinodylmethyl, cinnolinylmethyl, and benzothiophenylethyl. Specifically excluded from the scope of this term are compounds having adjacent ring O and/or S atoms.

An “arylene,” “heteroarylene,” or “heterocyclylene” group is a bivalent aryl, heteroaryl, or heterocyclyl group, respectively, as defined hereinabove, that is positioned between and serves to connect two other chemical groups.

As employed herein, when a moiety (e.g., cycloalkyl, aryl, heteroaryl, heterocyclyl, urea, etc.) is described as “optionally substituted” without expressly stating the substituents it is meant that the group optionally has from one to four, preferably from one to three, more preferably one or two, non-hydrogen substituents.

The term “halogen” or “halo” as employed herein refers to chlorine, bromine, fluorine, or iodine.

The term “haloalkyl” refers to an alkyl chain in which one or more hydrogens have been replaced by a halogen. Exemplary haloalkyls are trifluoromethyl, difluoromethyl, flurochloromethyl, chloromethyl, and fluoromethyl.

The term “hydroxyalkyl” refers to -alkylene-OH.

### EXAMPLE

The methods of the disclosure are illustrated further by the following examples, which is not to be construed as limiting the disclosure in scope or spirit to the specific procedures and compounds described in them.

### MRTX1719+KRAS^{G12D} Combination Study Procedure:

Immunodeficient female mice were implanted with human cancer models harboring homozygous deletion of the MTAP gene (MTAP^{DEL}) and a KRAS^{G12D} mutation. Mouse health was monitored daily, and caliper measurements began when tumors were palpable. Tumor volume measurements were determined utilizing the formula 0.5×L×W2 in which L refers to length and W refers to width of each tumor. When tumors reached approximately 200-300 mm³, animals were randomized to receive A) vehicle, B) a PRMT5 inhibitor administered orally (PO) formulated in 0.5% methylcellulose (4000 cps)/0.2% Tween80 in water, C) KRAS G12D inhibitor administered via intraperitoneal injection (IP) formulated in 10% Captisol in 50 mM Citrate buffer pH 5.0, or D) the PRMT5 inhibitor and KRAS G12D inhibitor. Tumor volumes were measured twice a week (n=5/treatment group). Tumor Growth Inhibition (% TGI) was calculated when the average final treated tumor volume was greater than initial treated tumor volume using the formula: (1-(Final Drug Treated Tumor Volume—Initial Drug Treated Tumor Volume)/(Final Vehicle Treated Tumor Volume -Initial Vehicle Treated Tumor Volume))*100. Percent Regression (% Regression) was calculated when the average tumor volume of final treated tumors was less than initial treated tumor volume using the formula: (−100%)*(1−((Final treated tumor volume)/(Initial treated tumor volume)).

### Example 1

This example was conducted in immunodeficient female nu/nu mice that were implanted with 5×10⁶ KP4 pancreatic cancer cells in 50% Matrigel. according to the study procedure described above. The PRMT5 inhibitor was MRTX1719 administered at 100 mg/kg once a day (QD). MRTX1719 used is a described herein.

The KRAS^{G12D} inhibitor used in this example was MRTX1133 administered at 30 mg/kg twice a day (BID). MRTX1133 used is as described herein.

Results are provided Table 1. The combination of MRTX1719 and MRTX1133 led to greater antitumor activity compared to either inhibitor alone in MTAP^{DEL} and this KRAS^{G12D} pancreatic tumor xenograft KP4 model.

TABLE 1

| | Monotherapy Treatment Groups | | | |
|---|---|---|---|---|
| | Vehicle 10% | MRTX1719 | MRTX1133 | Combination |
| | Captisol | 100 mg/kg | 30 mg/kg | MRTX1719 + |
| Study Day | QD IP | QD PO | BID IP | MRTX1133 |
| 0 | 275 | 277 | 276 | 259 |
| 3 | 420 | 431 | 202 | 210 |
| 7 | 757 | 616 | 132 | 148 |
| 10 | 880 | 716 | 99 | 83 |
| 14 | 1121 | 772 | 80 | 70 |
| 17 | 1477 | 887 | 91 | 88 |
| 21 | 1981 | 1222 | 144 | 73 |
| 24 | — | 1487 | 250 | 74 |
| 28 | — | — | 518 | 76 |
| 31 | — | — | 766 | 79 |
| 35 | — | — | 1064 | 78 |
| 38 | — | — | — | 53 |
| % TGI | — | 29.1 | 53.8 | — |
| % | — | — | — | −79.6 |
| Regression | | | | |

### Example 2

This example was conducted in immunodeficient female nu/nu mice that were implanted with 5×10⁶ SU8686 pancreatic cancer cells in 50% Matrigel. according to the study procedure described above. The PRMT5 inhibitor was MRTX1719 administered at 100 mg/kg once a day (QD). MRTX1719 used is as described herein.

The KRAS^{G12D} inhibitor used in this example was MRTX1133 administered at 30 mg/kg twice a day (BID). MRTX1133 used is as described herein.

Results are provided Table 2. The combination of MRTX1719 and MRTX1133 led to greater antitumor activity compared to either inhibitor alone in this MTAP^{DEL} and KRAS^{G12D} pancreatic tumor xenograft SU8686 model.

TABLE 2

| | Monotherapy Treatment Groups | | | |
|---|---|---|---|---|
| | Vehicle 10% | MRTX1719 | MRTX1133 | Combination |
| | Captisol | 100 mg/kg | 30 mg/kg | MRTX1719 + |
| Study Day | QD IP | QD PO | BID IP | MRTX1133 |
| −1 | 218 | 200 | 212 | 207 |
| 2 | 286 | 255 | 180 | 160 |
| 7 | 431 | 317 | 143 | 131 |
| 9 | 493 | 369 | 142 | 144 |
| 13 | 725 | 415 | 146 | 129 |
| 16 | 908 | 464 | 145 | 116 |
| 20 | 1062 | 467 | 164 | 108 |
| 23 | 1211 | 419 | 166 | 105 |
| 28 | 1288 | 396 | 151 | 111 |
| 31 | 1387 | 373 | 183 | 120 |
| 34 | — | 372 | 191 | 115 |
| 37 | — | 362 | 209 | 123 |
| 41 | — | 356 | 213 | 135 |
| 44 | — | 337 | 204 | 136 |
| % TGI | — | 88.3 | — | — |
| % | — | — | −4.2 | −34.5 |
| Regression | | | | |

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1. A method for treating cancer in a subject, the method comprising:
administering to the subject a therapeutically effective amount of a Kirsten rat sarcoma viral oncogene homolog (KRAS) glycine-to-aspartic acid at codon 12 (KRAS^{G12D}) inhibitor and a therapeutically effective amount of a methylthioadenosine (MTA)-cooperative protein arginine N-methyl transferase 5 (PRMT5) inhibitor.

2. The method of claim 1, wherein the cancer comprises methylthioadenosine phosphorylase (MTAP) gene homozygous deletion.

3. The method of claim 1, wherein the cancer comprises KRAS^{G12D} gene mutation.

4. The method of claim 2, wherein the cancer further comprise a cyclin-dependent kinase inhibitor 2A (CDKN2A) gene homozygous deletion.

5. The method of claim 1, wherein the cancer is lung cancer or pancreatic cancer.

6. The method of claim 1, wherein the cancer is lung cancer, such as non-small cell lung cancer (NSCLC).

7. The method of claim 1, wherein the cancer is pancreatic cancer.

8. The method of claim 1, wherein the KRAS^{G12D} inhibitor is 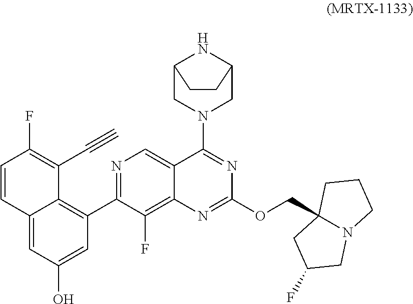 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]
or a pharmaceutically acceptable salt thereof.

9. The method of claim 1, wherein the PRMT5 inhibitor is: 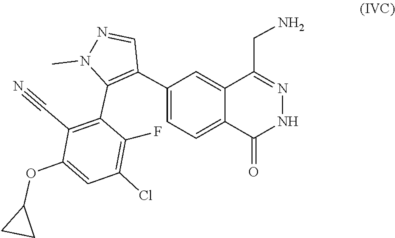 [SMILES: Cn1ncc(-c2ccc3c(=O)[nH]nc(CN)c3c2)c1-c1c(F)c(Cl)cc(OC2CC2)c1C#N]
or a pharmaceutically acceptable salt thereof.

10. The method of claim 1, wherein the PRMT5 inhibitor is: 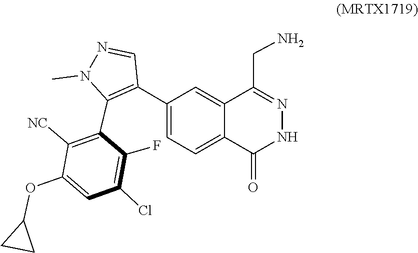
or a pharmaceutically acceptable salt thereof.

11. The method of claim 1, wherein the PRMT5 inhibitor is 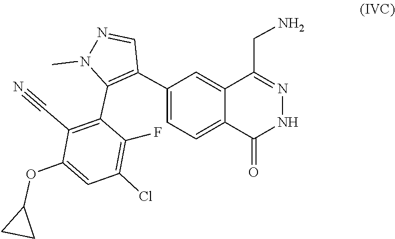 [SMILES: Cn1ncc(-c2ccc3c(=O)[nH]nc(CN)c3c2)c1-c1c(F)c(Cl)cc(OC2CC2)c1C#N]
or a pharmaceutically acceptable salt thereof, and the KRAS^{G12D} inhibitor is a compound of Formula 1 or a pharmaceutically acceptable salt thereof.

12. The method of claim 1, wherein the PRMT5 inhibitor is 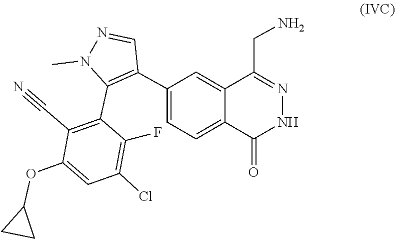 [SMILES: Cn1ncc(-c2ccc3c(=O)[nH]nc(CN)c3c2)c1-c1c(F)c(Cl)cc(OC2CC2)c1C#N]
or a pharmaceutically acceptable salt thereof, and the KRAS^{G12D} inhibitor is MRTX-1133 or a pharmaceutically acceptable salt thereof.

13. The method of claim 1, wherein the PRMT5 inhibitor is MRTX1719 or a pharmaceutically acceptable salt thereof, and the KRAS^{G}12D inhibitor is MRTX-1133 or a pharmaceutically acceptable salt thereof.

14. The method of claim 1, wherein the therapeutically effective amount of the PRMT5 inhibitor is in the range of about 0.01 to 300 mg/kg per day.

15. The method of claim 1, wherein the therapeutically effective amount of the PRMT5 inhibitor is in the range of about 0.1 to 100 mg/kg per day.

16. The method of claim 1, wherein the therapeutically effective amount of the PRMT5 inhibitor is less than 1% of, e.g., less than 10%, or less than 25%, or less than 50% of the clinically-established therapeutic amount.

17. The method of claim 14, wherein the therapeutically effective amount of the PRMT5 inhibitor is administered once daily.

18. The method of claim 1, wherein the therapeutically effective amount of the KRAS^{G12D} inhibitor is in the range of about 0.01 to 300 mg/kg per day.

19. The method of claim 1, wherein the therapeutically effective amount of the KRAS^{G12D} inhibitor is in the range of about 0.1 to 100 mg/kg per day.

20. The method of claim 1, wherein the therapeutically effective amount of the KRAS^{G12D} inhibitor is less than 1% of, e.g., less than 10%, or less than 25%, or less than 50% of the clinically-established therapeutic amount.

21. The method of claim 18, wherein the therapeutically effective amount of the KRAS^{G12D} is administered twice daily.

22. The method of claim 1, wherein the KRAS^{G12D} inhibitor and the PRMT5 inhibitor are administered sequentially.

23. The method of claim 1, wherein the KRAS^{G12D} inhibitor and the PRMT5 inhibitor are administered simultaneously.

24. The method of claim 1, wherein the subject previously received or completed a first-line chemotherapy.

25. The method of claim 1, wherein the subject did not previously received or complete a first-line chemotherapy.

26. The method of claim 24, wherein the first-line chemotherapy is platinum- and/or taxane-based chemotherapy.
